SEQUENCE LISTING (1145 sequences)

SEQ ID NO: 1 - protein, 9 aa, Artificial Sequence
  Synthetic Polypeptide
PFPQPELPY

SEQ ID NO: 2 - protein, 9 aa, Artificial Sequence
  Synthetic Polypeptide
PQPELPYPQ

SEQ ID NO: 3 - protein, 9 aa, Artificial Sequence
  Synthetic Polypeptide
PFPQPEQPF

SEQ ID NO: 4 - protein, 9 aa, Artificial Sequence
  Synthetic Polypeptide
PQPEQPFPW

SEQ ID NO: 5 - protein, 9 aa, Artificial Sequence
  Synthetic Polypeptide
EQPIPEQPQ

SEQ ID NO: 6 - protein, 9 aa, Artificial Sequence
  Synthetic Polypeptide
PIPEQPQPY

SEQ ID NO: 7 - protein, 9 aa, Artificial Sequence
  Synthetic Polypeptide
PFPQPEQPI

SEQ ID NO: 8 - protein, 9 aa, Artificial Sequence
  Synthetic Polypeptide
PQPEQPIPV

SEQ ID NO: 9 - protein, 9 aa, Artificial Sequence
  Synthetic Polypeptide
EQPIPVQPE

SEQ ID NO: 10 - protein, 9 aa, Artificial Sequence
  Synthetic Polypeptide
PFPQPEQPT

SEQ ID NO: 11 - protein, 9 aa, Artificial Sequence
  Synthetic Polypeptide
PQPEQPTPI

SEQ ID NO: 12 - protein, 9 aa, Artificial Sequence
  Synthetic Polypeptide
EQPTPIQPE

SEQ ID NO: 13 - protein, 9 aa, Artificial Sequence
  Synthetic Polypeptide
PQPEQPFPL

SEQ ID NO: 14 - protein, 9 aa, Artificial Sequence
  Synthetic Polypeptide
EQPFPLQPE

SEQ ID NO: 15 - protein, 9 aa, Artificial Sequence
  Synthetic Polypeptide
EGSFQPSQE

SEQ ID NO: 16 - protein, 9 aa, Artificial Sequence
  Synthetic Polypeptide
QGYYPTSPQ

SEQ ID NO: 17 - protein, 9 aa, Artificial Sequence
  Synthetic Polypeptide
EQPEQPFPE

SEQ ID NO: 18 - protein, 9 aa, Artificial Sequence
  Synthetic Polypeptide
PFSEQEQPV

SEQ ID NO: 19 - protein, 9 aa, Artificial Sequence
  Synthetic Polypeptide
PYPQPELPY

SEQ ID NO: 20 - protein, 9 aa, Artificial Sequence
  Synthetic Polypeptide
EQPFPEQPI

SEQ ID NO: 21 - protein, 9 aa, Artificial Sequence
  Synthetic Polypeptide
PFPEQPIPE

SEQ ID NO: 22 - protein, 9 aa, Artificial Sequence
  Synthetic Polypeptide
PYPEQPQPF

SEQ ID NO: 23 - protein, 9 aa, Artificial Sequence
  Synthetic Polypeptide
PQPYPEQPQ

SEQ ID NO: 24 - protein, 12 aa, Artificial Sequence
  Synthetic Polypeptide
PFPQPELPYPQP

SEQ ID NO: 25 - protein, 12 aa, Artificial Sequence
  Synthetic Polypeptide
PFPQPEQPFPWQ

SEQ ID NO: 26 - protein, 12 aa, Artificial Sequence
  Synthetic Polypeptide
EQPIPEQPQPYP

SEQ ID NO: 27 - protein, 12 aa, Artificial Sequence
  Synthetic Polypeptide
PFPQPEQPIPVQ

SEQ ID NO: 28 - protein, 12 aa, Artificial Sequence
  Synthetic Polypeptide
PEQPIPVQPEQS

SEQ ID NO: 29 - protein, 12 aa, Artificial Sequence
  Synthetic Polypeptide
PFPQPEQPTPIQ

SEQ ID NO: 30 - protein, 12 aa, Artificial Sequence
  Synthetic Polypeptide
PEQPTPIQPEQP

SEQ ID NO: 31 - protein, 12 aa, Artificial Sequence
  Synthetic Polypeptide
PFPQPEQPFPLQ

SEQ ID NO: 32 - protein, 12 aa, Artificial Sequence
  Synthetic Polypeptide
PEQPFPLQPEQP

SEQ ID NO: 33 - protein, 12 aa, Artificial Sequence
  Synthetic Polypeptide
GEGSFQPSQENP

SEQ ID NO: 34 - protein, 12 aa, Artificial Sequence
  Synthetic Polypeptide
QQGYYPTSPQQS

SEQ ID NO: 35 - protein, 12 aa, Artificial Sequence
  Synthetic Polypeptide
PEQPEQPFPEQP

SEQ ID NO: 36 - protein, 12 aa, Artificial Sequence
  Synthetic Polypeptide
PPFSEQEQPVLP

SEQ ID NO: 37 - protein, 12 aa, Artificial Sequence
  Synthetic Polypeptide
PYPQPELPYPQP

SEQ ID NO: 38 - protein, 12 aa, Artificial Sequence
  Synthetic Polypeptide
EQPFPEQPIPEQ

SEQ ID NO: 39 - protein, 12 aa, Artificial Sequence
  Synthetic Polypeptide
PQPYPEQPQPFP

SEQ ID NO: 40 - protein, 9 aa, Artificial Sequence
  Synthetic Polypeptide
PQPEQPFSQ

SEQ ID NO: 41 - protein, 9 aa, Artificial Sequence
  Synthetic Polypeptide
EQPFPEQPQ

SEQ ID NO: 42 - protein, 9 aa, Artificial Sequence
  Synthetic Polypeptide
PFPEQPEQI

SEQ ID NO: 43 - protein, 9 aa, Artificial Sequence
  Synthetic Polypeptide
PFSEQEQPV

SEQ ID NO: 44 - protein, 9 aa, Artificial Sequence
  Synthetic Polypeptide
PQPELPYPY

SEQ ID NO: 45 - protein, 9 aa, Artificial Sequence
  Synthetic Polypeptide
PQPEQPFSQ

SEQ ID NO: 46 - protein, 9 aa, Artificial Sequence
  Synthetic Polypeptide
PYPEQPQPF

SEQ ID NO: 47 - protein, 9 aa, Artificial Sequence
  Synthetic Polypeptide
EGSFQPSQE

SEQ ID NO: 48 - protein, 9 aa, Artificial Sequence
  Synthetic Polypeptide
QGYYPTSPQ

SEQ ID NO: 49 - protein, 9 aa, Artificial Sequence
  Synthetic Polypeptide
EQPEQPFPE

SEQ ID NO: 50 - protein, 9 aa, Artificial Sequence
  Synthetic Polypeptide
EQPFPEQPQ

SEQ ID NO: 51 - protein, 9 aa, Artificial Sequence
  Synthetic Polypeptide
PFPEQPEQI

SEQ ID NO: 52 - protein, 9 aa, Artificial Sequence
  Synthetic Polypeptide
PFSEQEQPV

SEQ ID NO: 53 - protein, 9 aa, Artificial Sequence
  Synthetic Polypeptide
EQPFPEQPI

SEQ ID NO: 54 - protein, 9 aa, Artificial Sequence
  Synthetic Polypeptide
PFPEQPIPE

SEQ ID NO: 55 - protein, 9 aa, Artificial Sequence
  Synthetic Polypeptide
PYPQPELPY

SEQ ID NO: 56 - protein, 9 aa, Artificial Sequence
  Synthetic Polypeptide
PQPELPYPY

SEQ ID NO: 57 - protein, 9 aa, Artificial Sequence
  Synthetic Polypeptide
PQPYPEQPQ

SEQ ID NO: 58 - protein, 9 aa, Artificial Sequence
  Synthetic Polypeptide
EQPFPEQPI

SEQ ID NO: 59 - protein, 9 aa, Artificial Sequence
  Synthetic Polypeptide
PFPEQPIPE

SEQ ID NO: 60 - protein, 9 aa, Artificial Sequence
  Synthetic Polypeptide
EQPIPEQPQ

SEQ ID NO: 61 - protein, 9 aa, Artificial Sequence
  Synthetic Polypeptide
PIPEQPQPY

SEQ ID NO: 62 - protein, 18 aa, Artificial Sequence
  Synthetic Polypeptide
PEQPFPEQPIPEQPQPYP

SEQ ID NO: 63 - protein, 9 aa, Artificial Sequence
  Synthetic Polypeptide
PFPQPEQPF

SEQ ID NO: 64 - protein, 9 aa, Artificial Sequence
  Synthetic Polypeptide
PQPEQPFSQ

SEQ ID NO: 65 - protein, 9 aa, Artificial Sequence
  Synthetic Polypeptide
PYPEQPQPF

SEQ ID NO: 66 - protein, 9 aa, Artificial Sequence
  Synthetic Polypeptide
PFPEQPEQI

SEQ ID NO: 67 - protein, 9 aa, Artificial Sequence
  Synthetic Polypeptide
EGSFQPSQE

SEQ ID NO: 68 - protein, 9 aa, Artificial Sequence
  Synthetic Polypeptide
QGYYPTSPQ

SEQ ID NO: 69 - protein, 9 aa, Artificial Sequence
  Synthetic Polypeptide
EQPEQPFPE

SEQ ID NO: 70 - protein, 9 aa, Artificial Sequence
  Synthetic Polypeptide
EQPFPEQPQ

SEQ ID NO: 71 - protein, 9 aa, Artificial Sequence
  Synthetic Polypeptide
PFSEQEQPV

SEQ ID NO: 72 - protein, 9 aa, Artificial Sequence
  Synthetic Polypeptide
EQPFPEQPI

SEQ ID NO: 73 - protein, 9 aa, Artificial Sequence
  Synthetic Polypeptide
PFPEQPIPE

SEQ ID NO: 74 - protein, 9 aa, Artificial Sequence
  Synthetic Polypeptide
EQPIPEQPQ

SEQ ID NO: 75 - protein, 9 aa, Artificial Sequence
  Synthetic Polypeptide
PIPEQPQPY

SEQ ID NO: 76 - protein, 9 aa, Artificial Sequence
  Synthetic Polypeptide
PQPELPYPQ

SEQ ID NO: 77 - protein, 9 aa, Artificial Sequence
  Synthetic Polypeptide
PYPQPELPY

SEQ ID NO: 78 - protein, 9 aa, Artificial Sequence
  Synthetic Polypeptide
PFPQPELPY

SEQ ID NO: 79 - protein, 9 aa, Artificial Sequence
  Synthetic Polypeptide
PQPELPYPY

SEQ ID NO: 80 - protein, 9 aa, Artificial Sequence
  Synthetic Polypeptide
EQPFPEQPI

SEQ ID NO: 81 - protein, 9 aa, Artificial Sequence
  Synthetic Polypeptide
PFPEQPIPE

SEQ ID NO: 82 - protein, 9 aa, Artificial Sequence
  Synthetic Polypeptide
PQPYPEQPQ

SEQ ID NO: 83 - protein, 9 aa, Artificial Sequence
  Synthetic Polypeptide
PYPEQPQPF

SEQ ID NO: 84 - protein, 15 aa, Artificial Sequence
  Synthetic Polypeptide
LQPFPQPELPYPQPQ

SEQ ID NO: 85 - protein, 14 aa, Artificial Sequence
  Synthetic Polypeptide
QPFPQPEQPFPWQP

SEQ ID NO: 86 - protein, 15 aa, Artificial Sequence
  Synthetic Polypeptide
PEQPIPEQPQPYPQQ

SEQ ID NO: 87 - protein, 17 aa, Artificial Sequence
  Synthetic Polypeptide
QPFPQPEQPIPVQPEQS

SEQ ID NO: 88 - protein, 17 aa, Artificial Sequence
  Synthetic Polypeptide
QPFPQPEQPTPIQPEQP

SEQ ID NO: 89 - protein, 17 aa, Artificial Sequence
  Synthetic Polypeptide
QPFPQPEQPFPLQPEQP

SEQ ID NO: 90 - protein, 13 aa, Artificial Sequence
  Synthetic Polypeptide
QPFPQPEQPFSQQ

SEQ ID NO: 91 - protein, 14 aa, Artificial Sequence
  Synthetic Polypeptide
PQPYPEQPQPFPQQ

SEQ ID NO: 92 - protein, 15 aa, Artificial Sequence
  Synthetic Polypeptide
QPFPEQPEQIIPQQP

SEQ ID NO: 93 - protein, 14 aa, Artificial Sequence
  Synthetic Polypeptide
SGEGSFQPSQENPQ

SEQ ID NO: 94 - protein, 14 aa, Artificial Sequence
  Synthetic Polypeptide
GQQGYYPTSPQQSG

SEQ ID NO: 95 - protein, 14 aa, Artificial Sequence
  Synthetic Polypeptide
PEQPEQPFPEQPQQ

SEQ ID NO: 96 - protein, 14 aa, Artificial Sequence
  Synthetic Polypeptide
QPPFSEQEQPVLPQ

SEQ ID NO: 97 - protein, 18 aa, Artificial Sequence
  Synthetic Polypeptide
PEQPFPEQPIPEQPQPYP

SEQ ID NO: 98 - protein, 14 aa, Artificial Sequence
  Synthetic Polypeptide
QPYPQPELPYPQPQ

SEQ ID NO: 99 - protein, 14 aa, Artificial Sequence
  Synthetic Polypeptide
QPFPQPELPYPYPQ

SEQ ID NO: 100 - protein, 15 aa, Artificial Sequence
  Synthetic Polypeptide
PQEQPFPEQPIPEQP

SEQ ID NO: 101 - protein, 15 aa, Artificial Sequence
  Synthetic Polypeptide
QPQPYPEQPQPFPQQ

SEQ ID NO: 102 - protein, 9 aa, Artificial Sequence
  Synthetic Polypeptide
PFPQPELPY

SEQ ID NO: 103 - protein, 9 aa, Artificial Sequence
  Synthetic Polypeptide
PQPELPYPQ

SEQ ID NO: 104 - protein, 9 aa, Artificial Sequence
  Synthetic Polypeptide
PFPQPEQPF

SEQ ID NO: 105 - protein, 9 aa, Artificial Sequence
  Synthetic Polypeptide
PQPEQPFPW

SEQ ID NO: 106 - protein, 9 aa, Artificial Sequence
  Synthetic Polypeptide
PIPEQPQPY

SEQ ID NO: 107 - protein, 10 aa, Artificial Sequence
  Synthetic Polypeptide
PFPQPEQPIP

SEQ ID NO: 108 - protein, 9 aa, Artificial Sequence
  Synthetic Polypeptide
EQPIPVQPE

SEQ ID NO: 109 - protein, 11 aa, Artificial Sequence
  Synthetic Polypeptide
PFPQPEQPTPI

SEQ ID NO: 110 - protein, 9 aa, Artificial Sequence
  Synthetic Polypeptide
EQPTPIQPE

SEQ ID NO: 111 - protein, 9 aa, Artificial Sequence
  Synthetic Polypeptide
PQPEQPFPL

SEQ ID NO: 112 - protein, 9 aa, Artificial Sequence
  Synthetic Polypeptide
EQPFPLQPE

SEQ ID NO: 113 - protein, 9 aa, Artificial Sequence
  Synthetic Polypeptide
PFPQPEQPF

SEQ ID NO: 114 - protein, 9 aa, Artificial Sequence
  Synthetic Polypeptide
PQPEQPFSQ

SEQ ID NO: 115 - protein, 9 aa, Artificial Sequence
  Synthetic Polypeptide
PYPEQPQPF

SEQ ID NO: 116 - protein, 11 aa, Artificial Sequence
  Synthetic Polypeptide
PFPEQPEQIIP

SEQ ID NO: 117 - protein, 9 aa, Artificial Sequence
  Synthetic Polypeptide
EGSFQPSQE

SEQ ID NO: 118 - protein, 9 aa, Artificial Sequence
  Synthetic Polypeptide
QGYYPTSPQ

SEQ ID NO: 119 - protein, 12 aa, Artificial Sequence
  Synthetic Polypeptide
EQPEQPFPEQPQ

SEQ ID NO: 120 - protein, 9 aa, Artificial Sequence
  Synthetic Polypeptide
PFSEQEQPV

SEQ ID NO: 121 - protein, 9 aa, Artificial Sequence
  Synthetic Polypeptide
EQPFPEQPI

SEQ ID NO: 122 - protein, 9 aa, Artificial Sequence
  Synthetic Polypeptide
PIPEQPQPY

SEQ ID NO: 123 - protein, 9 aa, Artificial Sequence
  Synthetic Polypeptide
PQPELPYPQ

SEQ ID NO: 124 - protein, 9 aa, Artificial Sequence
  Synthetic Polypeptide
PYPQPELPY

SEQ ID NO: 125 - protein, 9 aa, Artificial Sequence
  Synthetic Polypeptide
PFPQPELPY

SEQ ID NO: 126 - protein, 9 aa, Artificial Sequence
  Synthetic Polypeptide
PQPELPYPY

SEQ ID NO: 127 - protein, 9 aa, Artificial Sequence
  Synthetic Polypeptide
EQPFPEQPI

SEQ ID NO: 128 - protein, 15 aa, Artificial Sequence
  Synthetic Polypeptide
LQPFPQPELPYPQPQ

SEQ ID NO: 129 - protein, 14 aa, Artificial Sequence
  Synthetic Polypeptide
QPFPQPEQPFPWQP

SEQ ID NO: 130 - protein, 15 aa, Artificial Sequence
  Synthetic Polypeptide
PEQPIPEQPQPYPQQ

SEQ ID NO: 131 - protein, 17 aa, Artificial Sequence
  Synthetic Polypeptide
QPFPQPEQPIPVQPEQS

SEQ ID NO: 132 - protein, 17 aa, Artificial Sequence
  Synthetic Polypeptide
QPFPQPEQPTPIQPEQP

SEQ ID NO: 133 - protein, 17 aa, Artificial Sequence
  Synthetic Polypeptide
QPFPQPEQPFPLQPEQP

SEQ ID NO: 134 - protein, 13 aa, Artificial Sequence
  Synthetic Polypeptide
QPFPQPEQPFSQQ

SEQ ID NO: 135 - protein, 14 aa, Artificial Sequence
  Synthetic Polypeptide
PQPYPEQPQPFPQQ

SEQ ID NO: 136 - protein, 15 aa, Artificial Sequence
  Synthetic Polypeptide
QPFPEQPEQIIPQQP

SEQ ID NO: 137 - protein, 14 aa, Artificial Sequence
  Synthetic Polypeptide
SGEGSFQPSQENPQ

SEQ ID NO: 138 - protein, 14 aa, Artificial Sequence
  Synthetic Polypeptide
GQQGYYPTSPQQSG

SEQ ID NO: 139 - protein, 14 aa, Artificial Sequence
  Synthetic Polypeptide
PEQPEQPFPEQPQQ

SEQ ID NO: 140 - protein, 14 aa, Artificial Sequence
  Synthetic Polypeptide
QPPFSEQEQPVLPQ

SEQ ID NO: 141 - protein, 18 aa, Artificial Sequence
  Synthetic Polypeptide
PEQPFPEQPIPEQPQPYP

SEQ ID NO: 142 - protein, 14 aa, Artificial Sequence
  Synthetic Polypeptide
QPYPQPELPYPQPQ

SEQ ID NO: 143 - protein, 14 aa, Artificial Sequence
  Synthetic Polypeptide
QPFPQPELPYPYPQ

SEQ ID NO: 144 - protein, 9 aa, Artificial Sequence
  Synthetic Polypeptide
EQPFPEQPI

SEQ ID NO: 145 - protein, 9 aa, Artificial Sequence
  Synthetic Polypeptide
PFPQPELPY

SEQ ID NO: 146 - protein, 9 aa, Artificial Sequence
  Synthetic Polypeptide
PQPELPYPQ

SEQ ID NO: 147 - protein, 9 aa, Artificial Sequence
  Synthetic Polypeptide
PFPQPEQPF

SEQ ID NO: 148 - protein, 9 aa, Artificial Sequence
  Synthetic Polypeptide
PQPEQPFPW

SEQ ID NO: 149 - protein, 9 aa, Artificial Sequence
  Synthetic Polypeptide
EQPIPEQPQ

SEQ ID NO: 150 - protein, 9 aa, Artificial Sequence
  Synthetic Polypeptide
PIPEQPQPY

SEQ ID NO: 151 - protein, 8 aa, Artificial Sequence
  Synthetic Polypeptide
PFPQPEQP

SEQ ID NO: 152 - protein, 9 aa, Artificial Sequence
  Synthetic Polypeptide
PQPEQPIPV

SEQ ID NO: 153 - protein, 9 aa, Artificial Sequence
  Synthetic Polypeptide
EQPIPVQPE

SEQ ID NO: 154 - protein, 9 aa, Artificial Sequence
  Synthetic Polypeptide
PFPQPEQPT

SEQ ID NO: 155 - protein, 9 aa, Artificial Sequence
  Synthetic Polypeptide
PQPEQPTPI

SEQ ID NO: 156 - protein, 9 aa, Artificial Sequence
  Synthetic Polypeptide
EQPTPIQPE

SEQ ID NO: 157 - protein, 9 aa, Artificial Sequence
  Synthetic Polypeptide
PFPQPEQPF

SEQ ID NO: 158 - protein, 9 aa, Artificial Sequence
  Synthetic Polypeptide
PQPEQPFPL

SEQ ID NO: 159 - protein, 9 aa, Artificial Sequence
  Synthetic Polypeptide
EQPFPLQPE

SEQ ID NO: 160 - protein, 9 aa, Artificial Sequence
  Synthetic Polypeptide
PFPQPEQPF

SEQ ID NO: 161 - protein, 9 aa, Artificial Sequence
  Synthetic Polypeptide
PQPEQPFSQ

SEQ ID NO: 162 - protein, 9 aa, Artificial Sequence
  Synthetic Polypeptide
PYPEQPQPF

SEQ ID NO: 163 - protein, 9 aa, Artificial Sequence
  Synthetic Polypeptide
PFPEQPEQI

SEQ ID NO: 164 - protein, 9 aa, Artificial Sequence
  Synthetic Polypeptide
EGSFQPSQE

SEQ ID NO: 165 - protein, 9 aa, Artificial Sequence
  Synthetic Polypeptide
QGYYPTSPQ

SEQ ID NO: 166 - protein, 9 aa, Artificial Sequence
  Synthetic Polypeptide
EQPEQPFPE

SEQ ID NO: 167 - protein, 9 aa, Artificial Sequence
  Synthetic Polypeptide
EQPFPEQPQ

SEQ ID NO: 168 - protein, 9 aa, Artificial Sequence
  Synthetic Polypeptide
PFSEQEQPV

SEQ ID NO: 169 - protein, 9 aa, Artificial Sequence
  Synthetic Polypeptide
EQPFPEQPI

SEQ ID NO: 170 - protein, 9 aa, Artificial Sequence
  Synthetic Polypeptide
PFPEQPIPE

SEQ ID NO: 171 - protein, 9 aa, Artificial Sequence
  Synthetic Polypeptide
EQPIPEQPQ

SEQ ID NO: 172 - protein, 9 aa, Artificial Sequence
  Synthetic Polypeptide
PIPEQPQPY

SEQ ID NO: 173 - protein, 9 aa, Artificial Sequence
  Synthetic Polypeptide
PQPELPYPQ

SEQ ID NO: 174 - protein, 9 aa, Artificial Sequence
  Synthetic Polypeptide
PYPQPELPY

SEQ ID NO: 175 - protein, 9 aa, Artificial Sequence
  Synthetic Polypeptide
PFPQPELPY

SEQ ID NO: 176 - protein, 9 aa, Artificial Sequence
  Synthetic Polypeptide
PQPELPYPY

SEQ ID NO: 177 - protein, 9 aa, Artificial Sequence
  Synthetic Polypeptide
EQPFPEQPI

SEQ ID NO: 178 - protein, 9 aa, Artificial Sequence
  Synthetic Polypeptide
PFPEQPIPE

SEQ ID NO: 179 - protein, 9 aa, Artificial Sequence
  Synthetic Polypeptide
PQPYPEQPQ

SEQ ID NO: 180 - protein, 9 aa, Artificial Sequence
  Synthetic Polypeptide
PYPEQPQPF

SEQ ID NO: 181 - protein, 15 aa, Artificial Sequence
  Synthetic Polypeptide
LQPFPQPELPYPQPQ

SEQ ID NO: 182 - protein, 14 aa, Artificial Sequence
  Synthetic Polypeptide
QPFPQPEQPFPWQP

SEQ ID NO: 183 - protein, 15 aa, Artificial Sequence
  Synthetic Polypeptide
PEQPIPEQPQPYPQQ

SEQ ID NO: 184 - protein, 17 aa, Artificial Sequence
  Synthetic Polypeptide
QPFPQPEQPIPVQPEQS

SEQ ID NO: 185 - protein, 17 aa, Artificial Sequence
  Synthetic Polypeptide
QPFPQPEQPTPIQPEQP

SEQ ID NO: 186 - protein, 17 aa, Artificial Sequence
  Synthetic Polypeptide
QPFPQPEQPFPLQPEQP

SEQ ID NO: 187 - protein, 13 aa, Artificial Sequence
  Synthetic Polypeptide
QPFPQPEQPFSQQ

SEQ ID NO: 188 - protein, 14 aa, Artificial Sequence
  Synthetic Polypeptide
PQPYPEQPQPFPQQ

SEQ ID NO: 189 - protein, 15 aa, Artificial Sequence
  Synthetic Polypeptide
QPFPEQPEQIIPQQP

SEQ ID NO: 190 - protein, 14 aa, Artificial Sequence
  Synthetic Polypeptide
SGEGSFQPSQENPQ

SEQ ID NO: 191 - protein, 14 aa, Artificial Sequence
  Synthetic Polypeptide
GQQGYYPTSPQQSG

SEQ ID NO: 192 - protein, 14 aa, Artificial Sequence
  Synthetic Polypeptide
PEQPEQPFPEQPQQ

SEQ ID NO: 193 - protein, 14 aa, Artificial Sequence
  Synthetic Polypeptide
QPPFSEQEQPVLPQ

SEQ ID NO: 194 - protein, 18 aa, Artificial Sequence
  Synthetic Polypeptide
PEQPFPEQPIPEQPQPYP

SEQ ID NO: 195 - protein, 14 aa, Artificial Sequence
  Synthetic Polypeptide
QPYPQPELPYPQPQ

SEQ ID NO: 196 - protein, 14 aa, Artificial Sequence
  Synthetic Polypeptide
QPFPQPELPYPYPQ

SEQ ID NO: 197 - protein, 15 aa, Artificial Sequence
  Synthetic Polypeptide
PQEQPFPEQPIPEQP

SEQ ID NO: 198 - protein, 15 aa, Artificial Sequence
  Synthetic Polypeptide
QPQPYPEQPQPFPQQ

SEQ ID NO: 199 - protein, 9 aa, Artificial Sequence
  Synthetic Polypeptide
PFPQPELPY

SEQ ID NO: 200 - protein, 9 aa, Artificial Sequence
  Synthetic Polypeptide
PQPELPYPQ

SEQ ID NO: 201 - protein, 9 aa, Artificial Sequence
  Synthetic Polypeptide
PFPQPEQPF

SEQ ID NO: 202 - protein, 9 aa, Artificial Sequence
  Synthetic Polypeptide
PQPEQPFPW

SEQ ID NO: 203 - protein, 9 aa, Artificial Sequence
  Synthetic Polypeptide
PIPEQPQPY

SEQ ID NO: 204 - protein, 10 aa, Artificial Sequence
  Synthetic Polypeptide
PFPQPEQPIP

SEQ ID NO: 205 - protein, 9 aa, Artificial Sequence
  Synthetic Polypeptide
EQPIPVQPE

SEQ ID NO: 206 - protein, 11 aa, Artificial Sequence
  Synthetic Polypeptide
PFPQPEQPTPI

SEQ ID NO: 207 - protein, 9 aa, Artificial Sequence
  Synthetic Polypeptide
EQPTPIQPE

SEQ ID NO: 208 - protein, 9 aa, Artificial Sequence
  Synthetic Polypeptide
PQPEQPFPL

SEQ ID NO: 209 - protein, 9 aa, Artificial Sequence
  Synthetic Polypeptide
EQPFPLQPE

SEQ ID NO: 210 - protein, 9 aa, Artificial Sequence
  Synthetic Polypeptide
PFPQPEQPF

SEQ ID NO: 211 - protein, 9 aa, Artificial Sequence
  Synthetic Polypeptide
PQPEQPFSQ

SEQ ID NO: 212 - protein, 9 aa, Artificial Sequence
  Synthetic Polypeptide
PYPEQPQPF

SEQ ID NO: 213 - protein, 11 aa, Artificial Sequence
  Synthetic Polypeptide
PFPEQPEQIIP

SEQ ID NO: 214 - protein, 9 aa, Artificial Sequence
  Synthetic Polypeptide
EGSFQPSQE

SEQ ID NO: 215 - protein, 9 aa, Artificial Sequence
  Synthetic Polypeptide
QGYYPTSPQ

SEQ ID NO: 216 - protein, 12 aa, Artificial Sequence
  Synthetic Polypeptide
EQPEQPFPEQPQ

SEQ ID NO: 217 - protein, 9 aa, Artificial Sequence
  Synthetic Polypeptide
PFSEQEQPV

SEQ ID NO: 218 - protein, 9 aa, Artificial Sequence
  Synthetic Polypeptide
EQPFPEQPI

SEQ ID NO: 219 - protein, 9 aa, Artificial Sequence
  Synthetic Polypeptide
PIPEQPQPY

SEQ ID NO: 220 - protein, 9 aa, Artificial Sequence
  Synthetic Polypeptide
PQPELPYPQ

SEQ ID NO: 221 - protein, 9 aa, Artificial Sequence
  Synthetic Polypeptide
PYPQPELPY

SEQ ID NO: 222 - protein, 9 aa, Artificial Sequence
  Synthetic Polypeptide
PFPQPELPY

SEQ ID NO: 223 - protein, 9 aa, Artificial Sequence
  Synthetic Polypeptide
PQPELPYPY

SEQ ID NO: 224 - protein, 9 aa, Artificial Sequence
  Synthetic Polypeptide
EQPFPEQPI

SEQ ID NO: 225 - protein, 15 aa, Artificial Sequence
  Synthetic Polypeptide
LQPFPQPELPYPQPQ

SEQ ID NO: 226 - protein, 14 aa, Artificial Sequence
  Synthetic Polypeptide
QPFPQPEQPFPWQP

SEQ ID NO: 227 - protein, 15 aa, Artificial Sequence
  Synthetic Polypeptide
PEQPIPEQPQPYPQQ

SEQ ID NO: 228 - protein, 17 aa, Artificial Sequence
  Synthetic Polypeptide
QPFPQPEQPIPVQPEQS

SEQ ID NO: 229 - protein, 17 aa, Artificial Sequence
  Synthetic Polypeptide
QPFPQPEQPTPIQPEQP

SEQ ID NO: 230 - protein, 17 aa, Artificial Sequence
  Synthetic Polypeptide
QPFPQPEQPFPLQPEQP

SEQ ID NO: 231 - protein, 13 aa, Artificial Sequence
  Synthetic Polypeptide
QPFPQPEQPFSQQ

SEQ ID NO: 232 - protein, 14 aa, Artificial Sequence
  Synthetic Polypeptide
PQPYPEQPQPFPQQ

SEQ ID NO: 233 - protein, 15 aa, Artificial Sequence
  Synthetic Polypeptide
QPFPEQPEQIIPQQP

SEQ ID NO: 234 - protein, 14 aa, Artificial Sequence
  Synthetic Polypeptide
SGEGSFQPSQENPQ

SEQ ID NO: 235 - protein, 14 aa, Artificial Sequence
  Synthetic Polypeptide
GQQGYYPTSPQQSG

SEQ ID NO: 236 - protein, 14 aa, Artificial Sequence
  Synthetic Polypeptide
PEQPEQPFPEQPQQ

SEQ ID NO: 237 - protein, 14 aa, Artificial Sequence
  Synthetic Polypeptide
QPPFSEQEQPVLPQ

SEQ ID NO: 238 - protein, 18 aa, Artificial Sequence
  Synthetic Polypeptide
PEQPFPEQPIPEQPQPYP

SEQ ID NO: 239 - protein, 14 aa, Artificial Sequence
  Synthetic Polypeptide
QPYPQPELPYPQPQ

SEQ ID NO: 240 - protein, 14 aa, Artificial Sequence
  Synthetic Polypeptide
QPFPQPELPYPYPQ

SEQ ID NO: 241 - protein, 9 aa, Artificial Sequence
  Synthetic Polypeptide
EQPFPEQPI

SEQ ID NO: 242 - protein, 9 aa, Artificial Sequence
  Synthetic Polypeptide
PFPQPELPY

SEQ ID NO: 243 - protein, 9 aa, Artificial Sequence
  Synthetic Polypeptide
PQPELPYPQ

SEQ ID NO: 244 - protein, 9 aa, Artificial Sequence
  Synthetic Polypeptide
PFPQPEQPF

SEQ ID NO: 245 - protein, 9 aa, Artificial Sequence
  Synthetic Polypeptide
PQPEQPFPW

SEQ ID NO: 246 - protein, 9 aa, Artificial Sequence
  Synthetic Polypeptide
EQPIPEQPQ

SEQ ID NO: 247 - protein, 9 aa, Artificial Sequence
  Synthetic Polypeptide
PIPEQPQPY

SEQ ID NO: 248 - protein, 9 aa, Artificial Sequence
  Synthetic Polypeptide
PFPQPEQPI

SEQ ID NO: 249 - protein, 9 aa, Artificial Sequence
  Synthetic Polypeptide
PQPEQPIPV

SEQ ID NO: 250 - protein, 9 aa, Artificial Sequence
  Synthetic Polypeptide
EQPIPVQPE

SEQ ID NO: 251 - protein, 9 aa, Artificial Sequence
  Synthetic Polypeptide
PFPQPEQPT

SEQ ID NO: 252 - protein, 9 aa, Artificial Sequence
  Synthetic Polypeptide
PQPEQPTPI

SEQ ID NO: 253 - protein, 9 aa, Artificial Sequence
  Synthetic Polypeptide
EQPTPIQPE

SEQ ID NO: 254 - protein, 9 aa, Artificial Sequence
  Synthetic Polypeptide
PQPEQPFPL

SEQ ID NO: 255 - protein, 9 aa, Artificial Sequence
  Synthetic Polypeptide
EQPFPLQPE

SEQ ID NO: 256 - protein, 9 aa, Artificial Sequence
  Synthetic Polypeptide
EGSFQPSQE

SEQ ID NO: 257 - protein, 9 aa, Artificial Sequence
  Synthetic Polypeptide
QGYYPTSPQ

SEQ ID NO: 258 - protein, 9 aa, Artificial Sequence
  Synthetic Polypeptide
EQPEQPFPE

SEQ ID NO: 259 - protein, 9 aa, Artificial Sequence
  Synthetic Polypeptide
PFSEQEQPV

SEQ ID NO: 260 - protein, 9 aa, Artificial Sequence
  Synthetic Polypeptide
PYPQPELPY

SEQ ID NO: 261 - protein, 9 aa, Artificial Sequence
  Synthetic Polypeptide
EQPFPEQPI

SEQ ID NO: 262 - protein, 9 aa, Artificial Sequence
  Synthetic Polypeptide
PFPEQPIPE

SEQ ID NO: 263 - protein, 9 aa, Artificial Sequence
  Synthetic Polypeptide
PYPEQPQPF

SEQ ID NO: 264 - protein, 9 aa, Artificial Sequence
  Synthetic Polypeptide
PQPYPEQPQ

SEQ ID NO: 265 - protein, 9 aa, Artificial Sequence
  Synthetic Polypeptide
PFPQPELPY

SEQ ID NO: 266 - protein, 9 aa, Artificial Sequence
  Synthetic Polypeptide
PQPELPYPQ

SEQ ID NO: 267 - protein, 9 aa, Artificial Sequence
  Synthetic Polypeptide
PFPQPEQPF

SEQ ID NO: 268 - protein, 9 aa, Artificial Sequence
  Synthetic Polypeptide
PQPEQPFPW

SEQ ID NO: 269 - protein, 9 aa, Artificial Sequence
  Synthetic Polypeptide
EQPIPEQPQ

SEQ ID NO: 270 - protein, 9 aa, Artificial Sequence
  Synthetic Polypeptide
PIPEQPQPY

SEQ ID NO: 271 - protein, 9 aa, Artificial Sequence
  Synthetic Polypeptide
PFPQPEQPI

SEQ ID NO: 272 - protein, 9 aa, Artificial Sequence
  Synthetic Polypeptide
PQPEQPIPV

SEQ ID NO: 273 - protein, 9 aa, Artificial Sequence
  Synthetic Polypeptide
EQPIPVQPE

SEQ ID NO: 274 - protein, 9 aa, Artificial Sequence
  Synthetic Polypeptide
PFPQPEQPT

SEQ ID NO: 275 - protein, 9 aa, Artificial Sequence
  Synthetic Polypeptide
PQPEQPTPI

SEQ ID NO: 276 - protein, 9 aa, Artificial Sequence
  Synthetic Polypeptide
EQPTPIQPE

SEQ ID NO: 277 - protein, 9 aa, Artificial Sequence
  Synthetic Polypeptide
PQPEQPFPL

SEQ ID NO: 278 - protein, 9 aa, Artificial Sequence
  Synthetic Polypeptide
EQPFPLQPE

SEQ ID NO: 279 - protein, 9 aa, Artificial Sequence
  Synthetic Polypeptide
EGSFQPSQE

SEQ ID NO: 280 - protein, 9 aa, Artificial Sequence
  Synthetic Polypeptide
QGYYPTSPQ

SEQ ID NO: 281 - protein, 9 aa, Artificial Sequence
  Synthetic Polypeptide
EQPEQPFPE

SEQ ID NO: 282 - protein, 9 aa, Artificial Sequence
  Synthetic Polypeptide
PFSEQEQPV

SEQ ID NO: 283 - protein, 9 aa, Artificial Sequence
  Synthetic Polypeptide
PYPQPELPY

SEQ ID NO: 284 - protein, 9 aa, Artificial Sequence
  Synthetic Polypeptide
EQPFPEQPI

SEQ ID NO: 285 - protein, 9 aa, Artificial Sequence
  Synthetic Polypeptide
PFPEQPIPE

SEQ ID NO: 286 - protein, 9 aa, Artificial Sequence
  Synthetic Polypeptide
PYPEQPQPF

SEQ ID NO: 287 - protein, 9 aa, Artificial Sequence
  Synthetic Polypeptide
PQPYPEQPQ

SEQ ID NO: 288 - protein, 9 aa, Artificial Sequence
  Synthetic Polypeptide
PFPQPELPY

SEQ ID NO: 289 - protein, 9 aa, Artificial Sequence
  Synthetic Polypeptide
PQPELPYPQ

SEQ ID NO: 290 - protein, 9 aa, Artificial Sequence
  Synthetic Polypeptide
PFPQPEQPF

SEQ ID NO: 291 - protein, 9 aa, Artificial Sequence
  Synthetic Polypeptide
PQPEQPFPW

SEQ ID NO: 292 - protein, 9 aa, Artificial Sequence
  Synthetic Polypeptide
EQPIPEQPQ

SEQ ID NO: 293 - protein, 9 aa, Artificial Sequence
  Synthetic Polypeptide
PIPEQPQPY

SEQ ID NO: 294 - protein, 9 aa, Artificial Sequence
  Synthetic Polypeptide
PFPQPEQPI

SEQ ID NO: 295 - protein, 9 aa, Artificial Sequence
  Synthetic Polypeptide
PQPEQPIPV

SEQ ID NO: 296 - protein, 9 aa, Artificial Sequence
  Synthetic Polypeptide
EQPIPVQPE

SEQ ID NO: 297 - protein, 9 aa, Artificial Sequence
  Synthetic Polypeptide
PFPQPEQPT

SEQ ID NO: 298 - protein, 9 aa, Artificial Sequence
  Synthetic Polypeptide
PQPEQPTPI

SEQ ID NO: 299 - protein, 9 aa, Artificial Sequence
  Synthetic Polypeptide
EQPTPIQPE

SEQ ID NO: 300 - protein, 9 aa, Artificial Sequence
  Synthetic Polypeptide
PQPEQPFPL

SEQ ID NO: 301 - protein, 9 aa, Artificial Sequence
  Synthetic Polypeptide
EQPFPLQPE

SEQ ID NO: 302 - protein, 9 aa, Artificial Sequence
  Synthetic Polypeptide
EGSFQPSQE

SEQ ID NO: 303 - protein, 9 aa, Artificial Sequence
  Synthetic Polypeptide
QGYYPTSPQ

SEQ ID NO: 304 - protein, 9 aa, Artificial Sequence
  Synthetic Polypeptide
EQPEQPFPE

SEQ ID NO: 305 - protein, 9 aa, Artificial Sequence
  Synthetic Polypeptide
PFSEQEQPV

SEQ ID NO: 306 - protein, 9 aa, Artificial Sequence
  Synthetic Polypeptide
PYPQPELPY

SEQ ID NO: 307 - protein, 9 aa, Artificial Sequence
  Synthetic Polypeptide
EQPFPEQPI

SEQ ID NO: 308 - protein, 9 aa, Artificial Sequence
  Synthetic Polypeptide
PFPEQPIPE

SEQ ID NO: 309 - protein, 9 aa, Artificial Sequence
  Synthetic Polypeptide
PYPEQPQPF

SEQ ID NO: 310 - protein, 9 aa, Artificial Sequence
  Synthetic Polypeptide
PQPYPEQPQ

SEQ ID NO: 311 - protein, 12 aa, Artificial Sequence
  Synthetic Polypeptide
PFPQPELPYPQP

SEQ ID NO: 312 - protein, 12 aa, Artificial Sequence
  Synthetic Polypeptide
PFPQPEQPFPWQ

SEQ ID NO: 313 - protein, 12 aa, Artificial Sequence
  Synthetic Polypeptide
EQPIPEQPQPYP

SEQ ID NO: 314 - protein, 12 aa, Artificial Sequence
  Synthetic Polypeptide
PFPQPEQPIPVQ

SEQ ID NO: 315 - protein, 12 aa, Artificial Sequence
  Synthetic Polypeptide
PEQPIPVQPEQS

SEQ ID NO: 316 - protein, 12 aa, Artificial Sequence
  Synthetic Polypeptide
PFPQPEQPTPIQ

SEQ ID NO: 317 - protein, 12 aa, Artificial Sequence
  Synthetic Polypeptide
PEQPTPIQPEQP

SEQ ID NO: 318 - protein, 12 aa, Artificial Sequence
  Synthetic Polypeptide
PFPQPEQPFPLQ

SEQ ID NO: 319 - protein, 12 aa, Artificial Sequence
  Synthetic Polypeptide
PEQPFPLQPEQP

SEQ ID NO: 320 - protein, 12 aa, Artificial Sequence
  Synthetic Polypeptide
GEGSFQPSQENP

SEQ ID NO: 321 - protein, 12 aa, Artificial Sequence
  Synthetic Polypeptide
QQGYYPTSPQQS

SEQ ID NO: 322 - protein, 12 aa, Artificial Sequence
  Synthetic Polypeptide
PEQPEQPFPEQP

SEQ ID NO: 323 - protein, 12 aa, Artificial Sequence
  Synthetic Polypeptide
PPFSEQEQPVLP

SEQ ID NO: 324 - protein, 12 aa, Artificial Sequence
  Synthetic Polypeptide
PYPQPELPYPQP

SEQ ID NO: 325 - protein, 12 aa, Artificial Sequence
  Synthetic Polypeptide
EQPFPEQPIPEQ

SEQ ID NO: 326 - protein, 12 aa, Artificial Sequence
  Synthetic Polypeptide
PQPYPEQPQPFP

SEQ ID NO: 327 - DNA, 822 bp, Homo sapiens
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 328 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 329 - protein, 133 aa, Homo sapiens [sequence omitted]

SEQ ID NO: 330 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 331 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 332 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 333 - protein, 4 aa, Artificial Sequence
  Synthetic Polypeptide
PELP

SEQ ID NO: 334 - protein, 5 aa, Artificial Sequence
  Synthetic Polypeptide
PELPY

SEQ ID NO: 335 - protein, 7 aa, Artificial Sequence
  Synthetic Polypeptide
QPELPYP

SEQ ID NO: 336 - protein, 7 aa, Artificial Sequence
  Synthetic Polypeptide
PQPELPY

SEQ ID NO: 337 - protein, 7 aa, Artificial Sequence
  Synthetic Polypeptide
FPQPELP

SEQ ID NO: 338 - protein, 7 aa, Artificial Sequence
  Synthetic Polypeptide
PELPYPQ

SEQ ID NO: 339 - protein, 9 aa, Artificial Sequence
  Synthetic Polypeptide
FPQPELPYP

SEQ ID NO: 340 - protein, 9 aa, Artificial Sequence
  Synthetic Polypeptide
PYPQPELPY

SEQ ID NO: 341 - protein, 9 aa, Artificial Sequence
  Synthetic Polypeptide
PFPQPELPY

SEQ ID NO: 342 - protein, 9 aa, Artificial Sequence
  Synthetic Polypeptide
PQPELPYPQ

SEQ ID NO: 343 - protein, 9 aa, Artificial Sequence
  Synthetic Polypeptide
PFPQPEQPF

SEQ ID NO: 344 - protein, 9 aa, Artificial Sequence
  Synthetic Polypeptide
PQPEQPFPW

SEQ ID NO: 345 - protein, 9 aa, Artificial Sequence
  Synthetic Polypeptide
PIPEQPQPY

SEQ ID NO: 346 - protein, 9 aa, Artificial Sequence
  Synthetic Polypeptide
PQPELPYPQ

SEQ ID NO: 347 - protein, 9 aa, Artificial Sequence
  Synthetic Polypeptide
FRPEQPYPQ

SEQ ID NO: 348 - protein, 9 aa, Artificial Sequence
  Synthetic Polypeptide
PQQSFPEQQ

SEQ ID NO: 349 - protein, 9 aa, Artificial Sequence
  Synthetic Polypeptide
IQPEQPAQL

SEQ ID NO: 350 - protein, 9 aa, Artificial Sequence
  Synthetic Polypeptide
QQPEQPYPQ

SEQ ID NO: 351 - protein, 9 aa, Artificial Sequence
  Synthetic Polypeptide
SQPEQEFPQ

SEQ ID NO: 352 - protein, 9 aa, Artificial Sequence
  Synthetic Polypeptide
PQPEQEFPQ

SEQ ID NO: 353 - protein, 9 aa, Artificial Sequence
  Synthetic Polypeptide
QQPEQPFPQ

SEQ ID NO: 354 - protein, 9 aa, Artificial Sequence
  Synthetic Polypeptide
PQPEQPFCQ

SEQ ID NO: 355 - protein, 9 aa, Artificial Sequence
  Synthetic Polypeptide
QQPFPEQPQ

SEQ ID NO: 356 - protein, 9 aa, Artificial Sequence
  Synthetic Polypeptide
PFPQPEQPF

SEQ ID NO: 357 - protein, 9 aa, Artificial Sequence
  Synthetic Polypeptide
PQPEQPFPW

SEQ ID NO: 358 - protein, 9 aa, Artificial Sequence
  Synthetic Polypeptide
PFSEQEQPV

SEQ ID NO: 359 - protein, 9 aa, Artificial Sequence
  Synthetic Polypeptide
FSQQQESPF

SEQ ID NO: 360 - protein, 9 aa, Artificial Sequence
  Synthetic Polypeptide
PFPQPEQPF

SEQ ID NO: 361 - protein, 9 aa, Artificial Sequence
  Synthetic Polypeptide
PQPEQPFPQ

SEQ ID NO: 362 - protein, 9 aa, Artificial Sequence
  Synthetic Polypeptide
PIPEQPQPY

SEQ ID NO: 363 - protein, 9 aa, Artificial Sequence
  Synthetic Polypeptide
PFPQPEQPF

SEQ ID NO: 364 - protein, 9 aa, Artificial Sequence
  Synthetic Polypeptide
PQPEQPFPQ

SEQ ID NO: 365 - protein, 9 aa, Artificial Sequence
  Synthetic Polypeptide
PYPEQEEPF

SEQ ID NO: 366 - protein, 9 aa, Artificial Sequence
  Synthetic Polypeptide
PYPEQEQPF

SEQ ID NO: 367 - protein, 9 aa, Artificial Sequence
  Synthetic Polypeptide
PFSEQEQPV

SEQ ID NO: 368 - protein, 9 aa, Artificial Sequence
  Synthetic Polypeptide
EGSFQPSQE

SEQ ID NO: 369 - protein, 9 aa, Artificial Sequence
  Synthetic Polypeptide
EQPQQPFPQ

SEQ ID NO: 370 - protein, 9 aa, Artificial Sequence
  Synthetic Polypeptide
EQPQQPYPE

SEQ ID NO: 371 - protein, 10 aa, Artificial Sequence
  Synthetic Polypeptide
QQGYYPTSPQ

SEQ ID NO: 372 - protein, 9 aa, Artificial Sequence
  Synthetic Polypeptide
EGSFQPSQE

SEQ ID NO: 373 - protein, 9 aa, Artificial Sequence
  Synthetic Polypeptide
PQQSFPEQE

SEQ ID NO: 374 - protein, 9 aa, Artificial Sequence
  Synthetic Polypeptide
QGYYPTSPQ

SEQ ID NO: 375 - protein, 9 aa, Artificial Sequence
  Synthetic Polypeptide
PFPQPELPY

SEQ ID NO: 376 - protein, 9 aa, Artificial Sequence
  Synthetic Polypeptide
PQPELPYPQ

SEQ ID NO: 377 - protein, 9 aa, Artificial Sequence
  Synthetic Polypeptide
PFPQPEQPF

SEQ ID NO: 378 - protein, 9 aa, Artificial Sequence
  Synthetic Polypeptide
PQPEQPFPW

SEQ ID NO: 379 - protein, 9 aa, Artificial Sequence
  Synthetic Polypeptide
EQPIPEQPQ

SEQ ID NO: 380 - protein, 9 aa, Artificial Sequence
  Synthetic Polypeptide
PIPEQPQPY

SEQ ID NO: 381 - protein, 9 aa, Artificial Sequence
  Synthetic Polypeptide
PFPQPEQPI

SEQ ID NO: 382 - protein, 9 aa, Artificial Sequence
  Synthetic Polypeptide
PQPEQPIPV

SEQ ID NO: 383 - protein, 9 aa, Artificial Sequence
  Synthetic Polypeptide
EQPIPVQPE

SEQ ID NO: 384 - protein, 9 aa, Artificial Sequence
  Synthetic Polypeptide
PFPQPEQPT

SEQ ID NO: 385 - protein, 9 aa, Artificial Sequence
  Synthetic Polypeptide
PQPEQPTPI

SEQ ID NO: 386 - protein, 9 aa, Artificial Sequence
  Synthetic Polypeptide
EQPTPIQPE

SEQ ID NO: 387 - protein, 9 aa, Artificial Sequence
  Synthetic Polypeptide
PQPEQPFPL

SEQ ID NO: 388 - protein, 9 aa, Artificial Sequence
  Synthetic Polypeptide
EQPFPLQPE

SEQ ID NO: 389 - protein, 9 aa, Artificial Sequence
  Synthetic Polypeptide
PQPEQPFSQ

SEQ ID NO: 390 - protein, 9 aa, Artificial Sequence
  Synthetic Polypeptide
PYPEQPQPF

SEQ ID NO: 391 - protein, 9 aa, Artificial Sequence
  Synthetic Polypeptide
EGSFQPSQE

SEQ ID NO: 392 - protein, 9 aa, Artificial Sequence
  Synthetic Polypeptide
QGYYPTSPQ

SEQ ID NO: 393 - protein, 9 aa, Artificial Sequence
  Synthetic Polypeptide
EQPEQPFPE

SEQ ID NO: 394 - protein, 9 aa, Artificial Sequence
  Synthetic Polypeptide
EQPFPEQPQ

SEQ ID NO: 395 - protein, 9 aa, Artificial Sequence
  Synthetic Polypeptide
PFPEQPEQI

SEQ ID NO: 396 - protein, 9 aa, Artificial Sequence
  Synthetic Polypeptide
PFSEQEQPV

SEQ ID NO: 397 - protein, 9 aa, Artificial Sequence
  Synthetic Polypeptide
EQPFPEQPI

SEQ ID NO: 398 - protein, 9 aa, Artificial Sequence
  Synthetic Polypeptide
PFPEQPIPE

SEQ ID NO: 399 - protein, 9 aa, Artificial Sequence
  Synthetic Polypeptide
PYPQPELPY

SEQ ID NO: 400 - protein, 9 aa, Artificial Sequence
  Synthetic Polypeptide
PQPELPYPY

SEQ ID NO: 401 - protein, 9 aa, Artificial Sequence
  Synthetic Polypeptide
PQPYPEQPQ

SEQ ID NO: 402 - protein, 9 aa, Artificial Sequence
  Synthetic Polypeptide
PFPQPELPY

SEQ ID NO: 403 - protein, 9 aa, Artificial Sequence
  Synthetic Polypeptide
PQPELPYPQ

SEQ ID NO: 404 - protein, 9 aa, Artificial Sequence
  Synthetic Polypeptide
PFPQPEQPF

SEQ ID NO: 405 - protein, 9 aa, Artificial Sequence
  Synthetic Polypeptide
PQPEQPFPW

SEQ ID NO: 406 - protein, 9 aa, Artificial Sequence
  Synthetic Polypeptide
EQPIPEQPQ

SEQ ID NO: 407 - protein, 9 aa, Artificial Sequence
  Synthetic Polypeptide
PIPEQPQPY

SEQ ID NO: 408 - protein, 9 aa, Artificial Sequence
  Synthetic Polypeptide
PFPQPEQPI

SEQ ID NO: 409 - protein, 9 aa, Artificial Sequence
  Synthetic Polypeptide
PQPEQPIPV

SEQ ID NO: 410 - protein, 9 aa, Artificial Sequence
  Synthetic Polypeptide
EQPIPVQPE

SEQ ID NO: 411 - protein, 9 aa, Artificial Sequence
  Synthetic Polypeptide
PFPQPEQPT

SEQ ID NO: 412 - protein, 9 aa, Artificial Sequence
  Synthetic Polypeptide
PQPEQPTPI

SEQ ID NO: 413 - protein, 9 aa, Artificial Sequence
  Synthetic Polypeptide
EQPTPIQPE

SEQ ID NO: 414 - protein, 9 aa, Artificial Sequence
  Synthetic Polypeptide
PQPEQPFPL

SEQ ID NO: 415 - protein, 9 aa, Artificial Sequence
  Synthetic Polypeptide
EQPFPLQPE

SEQ ID NO: 416 - protein, 9 aa, Artificial Sequence
  Synthetic Polypeptide
PQPEQPFSQ

SEQ ID NO: 417 - protein, 9 aa, Artificial Sequence
  Synthetic Polypeptide
PYPEQPQPF

SEQ ID NO: 418 - protein, 9 aa, Artificial Sequence
  Synthetic Polypeptide
EGSFQPSQE

SEQ ID NO: 419 - protein, 9 aa, Artificial Sequence
  Synthetic Polypeptide
QGYYPTSPQ

SEQ ID NO: 420 - protein, 9 aa, Artificial Sequence
  Synthetic Polypeptide
EQPEQPFPE

SEQ ID NO: 421 - protein, 9 aa, Artificial Sequence
  Synthetic Polypeptide
EQPFPEQPQ

SEQ ID NO: 422 - protein, 9 aa, Artificial Sequence
  Synthetic Polypeptide
PFPEQPEQI

SEQ ID NO: 423 - protein, 9 aa, Artificial Sequence
  Synthetic Polypeptide
PFSEQEQPV

SEQ ID NO: 424 - protein, 9 aa, Artificial Sequence
  Synthetic Polypeptide
EQPFPEQPI

SEQ ID NO: 425 - protein, 9 aa, Artificial Sequence
  Synthetic Polypeptide
PFPEQPIPE

SEQ ID NO: 426 - protein, 9 aa, Artificial Sequence
  Synthetic Polypeptide
PYPQPELPY

SEQ ID NO: 427 - protein, 9 aa, Artificial Sequence
  Synthetic Polypeptide
PQPELPYPY

SEQ ID NO: 428 - protein, 9 aa, Artificial Sequence
  Synthetic Polypeptide
PQPYPEQPQ

SEQ ID NO: 429 - protein, 9 aa, Artificial Sequence
  Synthetic Polypeptide
PFPQPELPY

SEQ ID NO: 430 - protein, 9 aa, Artificial Sequence
  Synthetic Polypeptide
PQPELPYPQ

SEQ ID NO: 431 - protein, 9 aa, Artificial Sequence
  Synthetic Polypeptide
PFPQPEQPF

SEQ ID NO: 432 - protein, 9 aa, Artificial Sequence
  Synthetic Polypeptide
PQPEQPFPW

SEQ ID NO: 433 - protein, 9 aa, Artificial Sequence
  Synthetic Polypeptide
EQPIPEQPQ

SEQ ID NO: 434 - protein, 9 aa, Artificial Sequence
  Synthetic Polypeptide
PIPEQPQPY

SEQ ID NO: 435 - protein, 9 aa, Artificial Sequence
  Synthetic Polypeptide
PFPQPEQPI

SEQ ID NO: 436 - protein, 9 aa, Artificial Sequence
  Synthetic Polypeptide
PQPEQPIPV

SEQ ID NO: 437 - protein, 9 aa, Artificial Sequence
  Synthetic Polypeptide
EQPIPVQPE

SEQ ID NO: 438 - protein, 9 aa, Artificial Sequence
  Synthetic Polypeptide
PFPQPEQPT

SEQ ID NO: 439 - protein, 9 aa, Artificial Sequence
  Synthetic Polypeptide
PQPEQPTPI

SEQ ID NO: 440 - protein, 9 aa, Artificial Sequence
  Synthetic Polypeptide
EQPTPIQPE

SEQ ID NO: 441 - protein, 9 aa, Artificial Sequence
  Synthetic Polypeptide
PFPQPEQPF

SEQ ID NO: 442 - protein, 9 aa, Artificial Sequence
  Synthetic Polypeptide
PQPEQPFPL

SEQ ID NO: 443 - protein, 9 aa, Artificial Sequence
  Synthetic Polypeptide
EQPFPLQPE

SEQ ID NO: 444 - protein, 9 aa, Artificial Sequence
  Synthetic Polypeptide
PFPQPEQPF

SEQ ID NO: 445 - protein, 9 aa, Artificial Sequence
  Synthetic Polypeptide
PQPEQPFSQ

SEQ ID NO: 446 - protein, 9 aa, Artificial Sequence
  Synthetic Polypeptide
PYPEQPQPF

SEQ ID NO: 447 - protein, 9 aa, Artificial Sequence
  Synthetic Polypeptide
PFPEQPEQI

SEQ ID NO: 448 - protein, 9 aa, Artificial Sequence
  Synthetic Polypeptide
EGSFQPSQE

SEQ ID NO: 449 - protein, 9 aa, Artificial Sequence
  Synthetic Polypeptide
QGYYPTSPQ

SEQ ID NO: 450 - protein, 9 aa, Artificial Sequence
  Synthetic Polypeptide
EQPEQPFPE

SEQ ID NO: 451 - protein, 9 aa, Artificial Sequence
  Synthetic Polypeptide
EQPFPEQPQ

SEQ ID NO: 452 - protein, 9 aa, Artificial Sequence
  Synthetic Polypeptide
PFSEQEQPV

SEQ ID NO: 453 - protein, 9 aa, Artificial Sequence
  Synthetic Polypeptide
EQPFPEQPI

SEQ ID NO: 454 - protein, 9 aa, Artificial Sequence
  Synthetic Polypeptide
PFPEQPIPE

SEQ ID NO: 455 - protein, 9 aa, Artificial Sequence
  Synthetic Polypeptide
EQPIPEQPQ

SEQ ID NO: 456 - protein, 9 aa, Artificial Sequence
  Synthetic Polypeptide
PIPEQPQPY

SEQ ID NO: 457 - protein, 9 aa, Artificial Sequence
  Synthetic Polypeptide
PQPELPYPQ

SEQ ID NO: 458 - protein, 9 aa, Artificial Sequence
  Synthetic Polypeptide
PYPQPELPY

SEQ ID NO: 459 - protein, 9 aa, Artificial Sequence
  Synthetic Polypeptide
PFPQPELPY

SEQ ID NO: 460 - protein, 9 aa, Artificial Sequence
  Synthetic Polypeptide
PQPELPYPY

SEQ ID NO: 461 - protein, 9 aa, Artificial Sequence
  Synthetic Polypeptide
EQPFPEQPI

SEQ ID NO: 462 - protein, 9 aa, Artificial Sequence
  Synthetic Polypeptide
PFPEQPIPE

SEQ ID NO: 463 - protein, 9 aa, Artificial Sequence
  Synthetic Polypeptide
PQPYPEQPQ

SEQ ID NO: 464 - protein, 9 aa, Artificial Sequence
  Synthetic Polypeptide
PYPEQPQPF

SEQ ID NO: 465 - protein, 9 aa, Artificial Sequence
  Synthetic Polypeptide
PFPQPELPY

SEQ ID NO: 466 - protein, 9 aa, Artificial Sequence
  Synthetic Polypeptide
PQPELPYPQ

SEQ ID NO: 467 - protein, 9 aa, Artificial Sequence
  Synthetic Polypeptide
PFPQPEQPF

SEQ ID NO: 468 - protein, 9 aa, Artificial Sequence
  Synthetic Polypeptide
PQPEQPFPW

SEQ ID NO: 469 - protein, 9 aa, Artificial Sequence
  Synthetic Polypeptide
PIPEQPQPY

SEQ ID NO: 470 - protein, 10 aa, Artificial Sequence
  Synthetic Polypeptide
PFPQPEQPIP

SEQ ID NO: 471 - protein, 9 aa, Artificial Sequence
  Synthetic Polypeptide
EQPIPVQPE

SEQ ID NO: 472 - protein, 11 aa, Artificial Sequence
  Synthetic Polypeptide
PFPQPEQPTPI

SEQ ID NO: 473 - protein, 9 aa, Artificial Sequence
  Synthetic Polypeptide
EQPTPIQPE

SEQ ID NO: 474 - protein, 9 aa, Artificial Sequence
  Synthetic Polypeptide
PQPEQPFPL

SEQ ID NO: 475 - protein, 9 aa, Artificial Sequence
  Synthetic Polypeptide
EQPFPLQPE

SEQ ID NO: 476 - protein, 9 aa, Artificial Sequence
  Synthetic Polypeptide
PFPQPEQPF

SEQ ID NO: 477 - protein, 9 aa, Artificial Sequence
  Synthetic Polypeptide
PQPEQPFSQ

SEQ ID NO: 478 - protein, 9 aa, Artificial Sequence
  Synthetic Polypeptide
PYPEQPQPF

SEQ ID NO: 479 - protein, 11 aa, Artificial Sequence
  Synthetic Polypeptide
PFPEQPEQIIP

SEQ ID NO: 480 - protein, 9 aa, Artificial Sequence
  Synthetic Polypeptide
EGSFQPSQE

SEQ ID NO: 481 - protein, 9 aa, Artificial Sequence
  Synthetic Polypeptide
QGYYPTSPQ

SEQ ID NO: 482 - protein, 12 aa, Artificial Sequence
  Synthetic Polypeptide
EQPEQPFPEQPQ

SEQ ID NO: 483 - protein, 9 aa, Artificial Sequence
  Synthetic Polypeptide
PFSEQEQPV

SEQ ID NO: 484 - protein, 9 aa, Artificial Sequence
  Synthetic Polypeptide
EQPFPEQPI

SEQ ID NO: 485 - protein, 9 aa, Artificial Sequence
  Synthetic Polypeptide
PIPEQPQPY

SEQ ID NO: 486 - protein, 9 aa, Artificial Sequence
  Synthetic Polypeptide
PQPELPYPQ

SEQ ID NO: 487 - protein, 9 aa, Artificial Sequence
  Synthetic Polypeptide
PYPQPELPY

SEQ ID NO: 488 - protein, 9 aa, Artificial Sequence
  Synthetic Polypeptide
PFPQPELPY

SEQ ID NO: 489 - protein, 9 aa, Artificial Sequence
  Synthetic Polypeptide
PQPELPYPY

SEQ ID NO: 490 - protein, 9 aa, Artificial Sequence
  Synthetic Polypeptide
PFPQPELPY

SEQ ID NO: 491 - protein, 9 aa, Artificial Sequence
  Synthetic Polypeptide
PQPELPYPQ

SEQ ID NO: 492 - protein, 9 aa, Artificial Sequence
  Synthetic Polypeptide
PFPQPEQPF

SEQ ID NO: 493 - protein, 9 aa, Artificial Sequence
  Synthetic Polypeptide
PQPEQPFPW

SEQ ID NO: 494 - protein, 9 aa, Artificial Sequence
  Synthetic Polypeptide
PIPEQPQPY

SEQ ID NO: 495 - protein, 10 aa, Artificial Sequence
  Synthetic Polypeptide
PFPQPEQPIP

SEQ ID NO: 496 - protein, 9 aa, Artificial Sequence
  Synthetic Polypeptide
EQPIPVQPE

SEQ ID NO: 497 - protein, 11 aa, Artificial Sequence
  Synthetic Polypeptide
PFPQPEQPTPI

SEQ ID NO: 498 - protein, 9 aa, Artificial Sequence
  Synthetic Polypeptide
EQPTPIQPE

SEQ ID NO: 499 - protein, 9 aa, Artificial Sequence
  Synthetic Polypeptide
PQPEQPFPL

SEQ ID NO: 500 - protein, 9 aa, Artificial Sequence
  Synthetic Polypeptide
EQPFPLQPE

SEQ ID NO: 501 - protein, 9 aa, Artificial Sequence
  Synthetic Polypeptide
PFPQPEQPF

SEQ ID NO: 502 - protein, 9 aa, Artificial Sequence
  Synthetic Polypeptide
PQPEQPFSQ

SEQ ID NO: 503 - protein, 9 aa, Artificial Sequence
  Synthetic Polypeptide
PYPEQPQPF

SEQ ID NO: 504 - protein, 11 aa, Artificial Sequence
  Synthetic Polypeptide
PFPEQPEQIIP

SEQ ID NO: 505 - protein, 9 aa, Artificial Sequence
  Synthetic Polypeptide
EGSFQPSQE

SEQ ID NO: 506 - protein, 9 aa, Artificial Sequence
  Synthetic Polypeptide
QGYYPTSPQ

SEQ ID NO: 507 - protein, 12 aa, Artificial Sequence
  Synthetic Polypeptide
EQPEQPFPEQPQ

SEQ ID NO: 508 - protein, 9 aa, Artificial Sequence
  Synthetic Polypeptide
PFSEQEQPV

SEQ ID NO: 509 - protein, 9 aa, Artificial Sequence
  Synthetic Polypeptide
EQPFPEQPI

SEQ ID NO: 510 - protein, 9 aa, Artificial Sequence
  Synthetic Polypeptide
PIPEQPQPY

SEQ ID NO: 511 - protein, 9 aa, Artificial Sequence
  Synthetic Polypeptide
PQPELPYPQ

SEQ ID NO: 512 - protein, 9 aa, Artificial Sequence
  Synthetic Polypeptide
PYPQPELPY

SEQ ID NO: 513 - protein, 9 aa, Artificial Sequence
  Synthetic Polypeptide
PFPQPELPY

SEQ ID NO: 514 - protein, 9 aa, Artificial Sequence
  Synthetic Polypeptide
PQPELPYPY

SEQ ID NO: 515 - protein, 9 aa, Artificial Sequence
  Synthetic Polypeptide
EQPFPEQPI

SEQ ID NO: 516 - protein, 9 aa, Artificial Sequence
  Synthetic Polypeptide
PFPQPQLPY

SEQ ID NO: 517 - protein, 9 aa, Artificial Sequence
  Synthetic Polypeptide
PFPQPELPY

SEQ ID NO: 518 - protein, 9 aa, Artificial Sequence
  Synthetic Polypeptide
PFPQPELPY

SEQ ID NO: 519 - protein, 9 aa, Artificial Sequence
  Synthetic Polypeptide
PFPQPDLPY

SEQ ID NO: 520 - protein, 9 aa, Artificial Sequence
  Synthetic Polypeptide
PFPQPDLPY

SEQ ID NO: 521 - protein, 9 aa, Artificial Sequence
  Synthetic Polypeptide
PQPDLPYPQ

SEQ ID NO: 522 - protein, 9 aa, Artificial Sequence
  Synthetic Polypeptide
PFPQPDQPF

SEQ ID NO: 523 - protein, 9 aa, Artificial Sequence
  Synthetic Polypeptide
PQPDQPFPW

SEQ ID NO: 524 - protein, 9 aa, Artificial Sequence
  Synthetic Polypeptide
PIPDQPQPY

SEQ ID NO: 525 - protein, 15 aa, Artificial Sequence
  Synthetic Polypeptide
LQPFPQPDLPYPQPQ

SEQ ID NO: 526 - protein, 14 aa, Artificial Sequence
  Synthetic Polypeptide
QPFPQPDQPFPWQP

SEQ ID NO: 527 - protein, 15 aa, Artificial Sequence
  Synthetic Polypeptide
PQQPIPDQPQPYPQQ

SEQ ID NO: 528 - protein, 10 aa, Artificial Sequence
  Synthetic Polypeptide
PFPQPDQPIP

SEQ ID NO: 529 - protein, 9 aa, Artificial Sequence
  Synthetic Polypeptide
DQPIPVQPD

SEQ ID NO: 530 - protein, 11 aa, Artificial Sequence
  Synthetic Polypeptide
PFPQPDQPTPI

SEQ ID NO: 531 - protein, 9 aa, Artificial Sequence
  Synthetic Polypeptide
DQPTPIQPD

SEQ ID NO: 532 - protein, 9 aa, Artificial Sequence
  Synthetic Polypeptide
PQPDQPFPL

SEQ ID NO: 533 - protein, 9 aa, Artificial Sequence
  Synthetic Polypeptide
DQPFPLQPD

SEQ ID NO: 534 - protein, 9 aa, Artificial Sequence
  Synthetic Polypeptide
PFPQPDQPF

SEQ ID NO: 535 - protein, 9 aa, Artificial Sequence
  Synthetic Polypeptide
PQPDQPFSQ

SEQ ID NO: 536 - protein, 9 aa, Artificial Sequence
  Synthetic Polypeptide
PYPDQPQPF

SEQ ID NO: 537 - protein, 11 aa, Artificial Sequence
  Synthetic Polypeptide
PFPDQPDQIIP

SEQ ID NO: 538 - protein, 9 aa, Artificial Sequence
  Synthetic Polypeptide
DGSFQPSQD

SEQ ID NO: 539 - protein, 12 aa, Artificial Sequence
  Synthetic Polypeptide
DQPDQPFPDQPQ

SEQ ID NO: 540 - protein, 9 aa, Artificial Sequence
  Synthetic Polypeptide
PFSDQDQPV

SEQ ID NO: 541 - protein, 9 aa, Artificial Sequence
  Synthetic Polypeptide
DQPFPDQPI

SEQ ID NO: 542 - protein, 9 aa, Artificial Sequence
  Synthetic Polypeptide
PIPDQPQPY

SEQ ID NO: 543 - protein, 9 aa, Artificial Sequence
  Synthetic Polypeptide
PQPDLPYPQ

SEQ ID NO: 544 - protein, 9 aa, Artificial Sequence
  Synthetic Polypeptide
PYPQPDLPY

SEQ ID NO: 545 - protein, 9 aa, Artificial Sequence
  Synthetic Polypeptide
PFPQPDLPY

SEQ ID NO: 546 - protein, 9 aa, Artificial Sequence
  Synthetic Polypeptide
PQPDLPYPY

SEQ ID NO: 547 - protein, 4 aa, Artificial Sequence
  Synthetic Polypeptide
PQLP

SEQ ID NO: 548 - protein, 5 aa, Artificial Sequence
  Synthetic Polypeptide
PQLPY

SEQ ID NO: 549 - protein, 7 aa, Artificial Sequence
  Synthetic Polypeptide
QPQLPYP

SEQ ID NO: 550 - protein, 7 aa, Artificial Sequence
  Synthetic Polypeptide
PQPQLPY

SEQ ID NO: 551 - protein, 7 aa, Artificial Sequence
  Synthetic Polypeptide
FPQPQLP

SEQ ID NO: 552 - protein, 7 aa, Artificial Sequence
  Synthetic Polypeptide
PQLPYPQ

SEQ ID NO: 553 - protein, 9 aa, Artificial Sequence
  Synthetic Polypeptide
FPQPQLPYP

SEQ ID NO: 554 - protein, 9 aa, Artificial Sequence
  Synthetic Polypeptide
PYPQPQLPY

SEQ ID NO: 555 - protein, 9 aa, Artificial Sequence
  Synthetic Polypeptide
PFPQPQLPY

SEQ ID NO: 556 - protein, 9 aa, Artificial Sequence
  Synthetic Polypeptide
PQPQLPYPQ

SEQ ID NO: 557 - protein, 9 aa, Artificial Sequence
  Synthetic Polypeptide
PFPQPQQPF

SEQ ID NO: 558 - protein, 9 aa, Artificial Sequence
  Synthetic Polypeptide
PQPQQPFPW

SEQ ID NO: 559 - protein, 9 aa, Artificial Sequence
  Synthetic Polypeptide
PIPQQPQPY

SEQ ID NO: 560 - protein, 15 aa, Artificial Sequence
  Synthetic Polypeptide
LQPFPQPQLPYPQPQ

SEQ ID NO: 561 - protein, 14 aa, Artificial Sequence
  Synthetic Polypeptide
QPFPQPQQPFPWQP

SEQ ID NO: 562 - protein, 15 aa, Artificial Sequence
  Synthetic Polypeptide
PEQPIPQQPQPYPQQ

SEQ ID NO: 563 - protein, 9 aa, Artificial Sequence
  Synthetic Polypeptide
PQPQLPYPQ

SEQ ID NO: 564 - protein, 9 aa, Artificial Sequence
  Synthetic Polypeptide
FRPQQPYPQ

SEQ ID NO: 565 - protein, 9 aa, Artificial Sequence
  Synthetic Polypeptide
PQQSFPQQQ

SEQ ID NO: 566 - protein, 9 aa, Artificial Sequence
  Synthetic Polypeptide
IQPQQPAQL

SEQ ID NO: 567 - protein, 9 aa, Artificial Sequence
  Synthetic Polypeptide
QQPQQPYPQ

SEQ ID NO: 568 - protein, 9 aa, Artificial Sequence
  Synthetic Polypeptide
SQPQQQFPQ

SEQ ID NO: 569 - protein, 9 aa, Artificial Sequence
  Synthetic Polypeptide
PQPQQQFPQ

SEQ ID NO: 570 - protein, 9 aa, Artificial Sequence
  Synthetic Polypeptide
QQPQQPFPQ

SEQ ID NO: 571 - protein, 9 aa, Artificial Sequence
  Synthetic Polypeptide
PQPQQPFCQ

SEQ ID NO: 572 - protein, 9 aa, Artificial Sequence
  Synthetic Polypeptide
QQPFPQQPQ

SEQ ID NO: 573 - protein, 9 aa, Artificial Sequence
  Synthetic Polypeptide
PFPQPQQPF

SEQ ID NO: 574 - protein, 9 aa, Artificial Sequence
  Synthetic Polypeptide
PQPQQPFPW

SEQ ID NO: 575 - protein, 9 aa, Artificial Sequence
  Synthetic Polypeptide
PFSQQQQPV

SEQ ID NO: 576 - protein, 9 aa, Artificial Sequence
  Synthetic Polypeptide
FSQQQQSPF

SEQ ID NO: 577 - protein, 9 aa, Artificial Sequence
  Synthetic Polypeptide
PFPQPQQPF

SEQ ID NO: 578 - protein, 9 aa, Artificial Sequence
  Synthetic Polypeptide
PQPQQPFPQ

SEQ ID NO: 579 - protein, 9 aa, Artificial Sequence
  Synthetic Polypeptide
PIPQQPQPY

SEQ ID NO: 580 - protein, 9 aa, Artificial Sequence
  Synthetic Polypeptide
PFPQPQQPF

SEQ ID NO: 581 - protein, 9 aa, Artificial Sequence
  Synthetic Polypeptide
PQPQQPFPQ

SEQ ID NO: 582 - protein, 9 aa, Artificial Sequence
  Synthetic Polypeptide
PYPEQQEPF

SEQ ID NO: 583 - protein, 9 aa, Artificial Sequence
  Synthetic Polypeptide
PYPEQQQPF

SEQ ID NO: 584 - protein, 9 aa, Artificial Sequence
  Synthetic Polypeptide
PFSQQQQPV

SEQ ID NO: 585 - protein, 9 aa, Artificial Sequence
  Synthetic Polypeptide
QGSFQPSQQ

SEQ ID NO: 586 - protein, 9 aa, Artificial Sequence
  Synthetic Polypeptide
QQPQQPFPQ

SEQ ID NO: 587 - protein, 9 aa, Artificial Sequence
  Synthetic Polypeptide
QQPQQPYPQ

SEQ ID NO: 588 - protein, 10 aa, Artificial Sequence
  Synthetic Polypeptide
QQGYYPTSPQ

SEQ ID NO: 589 - protein, 9 aa, Artificial Sequence
  Synthetic Polypeptide
QGSFQPSQQ

SEQ ID NO: 590 - protein, 9 aa, Artificial Sequence
  Synthetic Polypeptide
PQQSFPQQQ

SEQ ID NO: 591 - protein, 9 aa, Artificial Sequence
  Synthetic Polypeptide
QGYYPTSPQ

SEQ ID NO: 592 - protein, 15 aa, Artificial Sequence
  Synthetic Polypeptide
LQPFPQPELPYPQPQ

SEQ ID NO: 593 - protein, 14 aa, Artificial Sequence
  Synthetic Polypeptide
QPFPQPQQPFPWQP

SEQ ID NO: 594 - protein, 15 aa, Artificial Sequence
  Synthetic Polypeptide
PQQPIPQQPQPYPQQ

SEQ ID NO: 595 - protein, 10 aa, Artificial Sequence
  Synthetic Polypeptide
PFPQPEQPIP

SEQ ID NO: 596 - protein, 9 aa, Artificial Sequence
  Synthetic Polypeptide
EQPIPVQPE

SEQ ID NO: 597 - protein, 11 aa, Artificial Sequence
  Synthetic Polypeptide
PFPQPEQPTPI

SEQ ID NO: 598 - protein, 9 aa, Artificial Sequence
  Synthetic Polypeptide
EQPTPIQPE

SEQ ID NO: 599 - protein, 9 aa, Artificial Sequence
  Synthetic Polypeptide
PQPEQPFPL

SEQ ID NO: 600 - protein, 9 aa, Artificial Sequence
  Synthetic Polypeptide
EQPFPLQPE

SEQ ID NO: 601 - protein, 9 aa, Artificial Sequence
  Synthetic Polypeptide
PFPQPEQPF

SEQ ID NO: 602 - protein, 9 aa, Artificial Sequence
  Synthetic Polypeptide
PQPEQPFSQ

SEQ ID NO: 603 - protein, 9 aa, Artificial Sequence
  Synthetic Polypeptide
PYPEQPQPF

SEQ ID NO: 604 - protein, 11 aa, Artificial Sequence
  Synthetic Polypeptide
PFPEQPEQIIP

SEQ ID NO: 605 - protein, 9 aa, Artificial Sequence
  Synthetic Polypeptide
EGSFQPSQE

SEQ ID NO: 606 - protein, 12 aa, Artificial Sequence
  Synthetic Polypeptide
EQPEQPFPEQPQ

SEQ ID NO: 607 - protein, 9 aa, Artificial Sequence
  Synthetic Polypeptide
PFSEQEQPV

SEQ ID NO: 608 - protein, 9 aa, Artificial Sequence
  Synthetic Polypeptide
EQPFPEQPI

SEQ ID NO: 609 - protein, 9 aa, Artificial Sequence
  Synthetic Polypeptide
PIPEQPQPY

SEQ ID NO: 610 - protein, 9 aa, Artificial Sequence
  Synthetic Polypeptide
PQPELPYPQ

SEQ ID NO: 611 - protein, 9 aa, Artificial Sequence
  Synthetic Polypeptide
PYPQPELPY

SEQ ID NO: 612 - protein, 9 aa, Artificial Sequence
  Synthetic Polypeptide
PFPQPELPY

SEQ ID NO: 613 - protein, 9 aa, Artificial Sequence
  Synthetic Polypeptide
PQPELPYPY

SEQ ID NO: 614 - protein, 15 aa, Artificial Sequence
  Synthetic Polypeptide
LQPFPQPQLPYPQPQ

SEQ ID NO: 615 - protein, 14 aa, Artificial Sequence
  Synthetic Polypeptide
QPFPQPQQPFPWQP

SEQ ID NO: 616 - protein, 15 aa, Artificial Sequence
  Synthetic Polypeptide
PQQPIPQQPQPYPQQ

SEQ ID NO: 617 - protein, 17 aa, Artificial Sequence
  Synthetic Polypeptide
QPFPQPQQPIPVQPQQS

SEQ ID NO: 618 - protein, 17 aa, Artificial Sequence
  Synthetic Polypeptide
QPFPQPQQPTPIQPQQP

SEQ ID NO: 619 - protein, 17 aa, Artificial Sequence
  Synthetic Polypeptide
QPFPQPQQPFPLQPQQP

SEQ ID NO: 620 - protein, 13 aa, Artificial Sequence
  Synthetic Polypeptide
QPFPQPQQPFSQQ

SEQ ID NO: 621 - protein, 14 aa, Artificial Sequence
  Synthetic Polypeptide
PQPYPQQPQPFPQQ

SEQ ID NO: 622 - protein, 15 aa, Artificial Sequence
  Synthetic Polypeptide
QPFPEQPQQIIPQQP

SEQ ID NO: 623 - protein, 14 aa, Artificial Sequence
  Synthetic Polypeptide
SGEGSFQPSQQNPQ

SEQ ID NO: 624 - protein, 14 aa, Artificial Sequence
  Synthetic Polypeptide
PQQPQQPFPQQPQQ

SEQ ID NO: 625 - protein, 14 aa, Artificial Sequence
  Synthetic Polypeptide
QPPFSQQQQPVLPQ

SEQ ID NO: 626 - protein, 18 aa, Artificial Sequence
  Synthetic Polypeptide
PQQPFPQQPIPQQPQPYP

SEQ ID NO: 627 - protein, 14 aa, Artificial Sequence
  Synthetic Polypeptide
QPYPQPQLPYPQPQ

SEQ ID NO: 628 - protein, 14 aa, Artificial Sequence
  Synthetic Polypeptide
QPFPQPQLPYPYPQ

SEQ ID NO: 629 - protein, 9 aa, Artificial Sequence
  Synthetic Polypeptide
FPQPELPYP

SEQ ID NO: 630 - protein, 17 aa, Artificial Sequence
  Synthetic Polypeptide
QLQPFPQPELPYPQPQS

SEQ ID NO: 631 - protein, 9 aa, Artificial Sequence
  Synthetic Polypeptide
PFPQPELPY

SEQ ID NO: 632 - protein, 9 aa, Artificial Sequence
  Synthetic Polypeptide
PQPELPYPQ

SEQ ID NO: 633 - protein, 9 aa, Artificial Sequence
  Synthetic Polypeptide
PFPQPEQPF

SEQ ID NO: 634 - protein, 9 aa, Artificial Sequence
  Synthetic Polypeptide
PQPEQPFPW

SEQ ID NO: 635 - protein, 9 aa, Artificial Sequence
  Synthetic Polypeptide
EQPIPEQPQ

SEQ ID NO: 636 - protein, 9 aa, Artificial Sequence
  Synthetic Polypeptide
PIPEQPQPY

SEQ ID NO: 637 - protein, 9 aa, Artificial Sequence
  Synthetic Polypeptide
PFPQPEQPI

SEQ ID NO: 638 - protein, 9 aa, Artificial Sequence
  Synthetic Polypeptide
PQPEQPIPV

SEQ ID NO: 639 - protein, 9 aa, Artificial Sequence
  Synthetic Polypeptide
EQPIPVQPE

SEQ ID NO: 640 - protein, 9 aa, Artificial Sequence
  Synthetic Polypeptide
PFPQPEQPT

SEQ ID NO: 641 - protein, 9 aa, Artificial Sequence
  Synthetic Polypeptide
PQPEQPTPI

SEQ ID NO: 642 - protein, 9 aa, Artificial Sequence
  Synthetic Polypeptide
EQPTPIQPE

SEQ ID NO: 643 - protein, 9 aa, Artificial Sequence
  Synthetic Polypeptide
PQPEQPFPL

SEQ ID NO: 644 - protein, 9 aa, Artificial Sequence
  Synthetic Polypeptide
EQPFPLQPE

SEQ ID NO: 645 - protein, 9 aa, Artificial Sequence
  Synthetic Polypeptide
PQPEQPFSQ

SEQ ID NO: 646 - protein, 9 aa, Artificial Sequence
  Synthetic Polypeptide
PYPEQPQPF

SEQ ID NO: 647 - protein, 9 aa, Artificial Sequence
  Synthetic Polypeptide
EGSFQPSQE

SEQ ID NO: 648 - protein, 9 aa, Artificial Sequence
  Synthetic Polypeptide
QGYYPTSPQ

SEQ ID NO: 649 - protein, 9 aa, Artificial Sequence
  Synthetic Polypeptide
EQPEQPFPE

SEQ ID NO: 650 - protein, 9 aa, Artificial Sequence
  Synthetic Polypeptide
EQPFPEQPQ

SEQ ID NO: 651 - protein, 9 aa, Artificial Sequence
  Synthetic Polypeptide
PFPEQPEQI

SEQ ID NO: 652 - protein, 9 aa, Artificial Sequence
  Synthetic Polypeptide
PFSEQEQPV

SEQ ID NO: 653 - protein, 9 aa, Artificial Sequence
  Synthetic Polypeptide
EQPFPEQPI

SEQ ID NO: 654 - protein, 9 aa, Artificial Sequence
  Synthetic Polypeptide
PFPEQPIPE

SEQ ID NO: 655 - protein, 9 aa, Artificial Sequence
  Synthetic Polypeptide
PYPQPELPY

SEQ ID NO: 656 - protein, 9 aa, Artificial Sequence
  Synthetic Polypeptide
PQPELPYPY

SEQ ID NO: 657 - protein, 9 aa, Artificial Sequence
  Synthetic Polypeptide
PQPYPEQPQ

SEQ ID NO: 658 - protein, 9 aa, Artificial Sequence
  Synthetic Polypeptide
PFPQPELPY

SEQ ID NO: 659 - protein, 9 aa, Artificial Sequence
  Synthetic Polypeptide
PQPELPYPQ

SEQ ID NO: 660 - protein, 9 aa, Artificial Sequence
  Synthetic Polypeptide
PFPQPEQPF

SEQ ID NO: 661 - protein, 9 aa, Artificial Sequence
  Synthetic Polypeptide
PQPEQPFPW

SEQ ID NO: 662 - protein, 9 aa, Artificial Sequence
  Synthetic Polypeptide
PIPEQPQPY

SEQ ID NO: 663 - protein, 10 aa, Artificial Sequence
  Synthetic Polypeptide
PFPQPEQPIP

SEQ ID NO: 664 - protein, 9 aa, Artificial Sequence
  Synthetic Polypeptide
EQPIPVQPE

SEQ ID NO: 665 - protein, 11 aa, Artificial Sequence
  Synthetic Polypeptide
PFPQPEQPTPI

SEQ ID NO: 666 - protein, 9 aa, Artificial Sequence
  Synthetic Polypeptide
EQPTPIQPE

SEQ ID NO: 667 - protein, 9 aa, Artificial Sequence
  Synthetic Polypeptide
PQPEQPFPL

SEQ ID NO: 668 - protein, 9 aa, Artificial Sequence
  Synthetic Polypeptide
EQPFPLQPE

SEQ ID NO: 669 - protein, 9 aa, Artificial Sequence
  Synthetic Polypeptide
PFPQPEQPF

SEQ ID NO: 670 - protein, 9 aa, Artificial Sequence
  Synthetic Polypeptide
PQPEQPFSQ

SEQ ID NO: 671 - protein, 9 aa, Artificial Sequence
  Synthetic Polypeptide
PYPEQPQPF

SEQ ID NO: 672 - protein, 11 aa, Artificial Sequence
  Synthetic Polypeptide
PFPEQPEQIIP

SEQ ID NO: 673 - protein, 9 aa, Artificial Sequence
  Synthetic Polypeptide
EGSFQPSQE

SEQ ID NO: 674 - protein, 9 aa, Artificial Sequence
  Synthetic Polypeptide
QGYYPTSPQ

SEQ ID NO: 675 - protein, 12 aa, Artificial Sequence
  Synthetic Polypeptide
EQPEQPFPEQPQ

SEQ ID NO: 676 - protein, 9 aa, Artificial Sequence
  Synthetic Polypeptide
PFSEQEQPV

SEQ ID NO: 677 - protein, 9 aa, Artificial Sequence
  Synthetic Polypeptide
EQPFPEQPI

SEQ ID NO: 678 - protein, 9 aa, Artificial Sequence
  Synthetic Polypeptide
PIPEQPQPY

SEQ ID NO: 679 - protein, 9 aa, Artificial Sequence
  Synthetic Polypeptide
PQPELPYPQ

SEQ ID NO: 680 - protein, 9 aa, Artificial Sequence
  Synthetic Polypeptide
PYPQPELPY

SEQ ID NO: 681 - protein, 9 aa, Artificial Sequence
  Synthetic Polypeptide
PFPQPELPY

SEQ ID NO: 682 - protein, 9 aa, Artificial Sequence
  Synthetic Polypeptide
PQPELPYPY

SEQ ID NO: 683 - protein, 9 aa, Artificial Sequence
  Synthetic Polypeptide
EQPFPEQPI

SEQ ID NO: 684 - protein, 9 aa, Artificial Sequence
  Synthetic Polypeptide
PFPQPELPY

SEQ ID NO: 685 - protein, 9 aa, Artificial Sequence
  Synthetic Polypeptide
PQPELPYPQ

SEQ ID NO: 686 - protein, 9 aa, Artificial Sequence
  Synthetic Polypeptide
PFPQPEQPF

SEQ ID NO: 687 - protein, 9 aa, Artificial Sequence
  Synthetic Polypeptide
PQPEQPFPW

SEQ ID NO: 688 - protein, 9 aa, Artificial Sequence
  Synthetic Polypeptide
EQPIPEQPQ

SEQ ID NO: 689 - protein, 9 aa, Artificial Sequence
  Synthetic Polypeptide
PIPEQPQPY

SEQ ID NO: 690 - protein, 9 aa, Artificial Sequence
  Synthetic Polypeptide
PFPQPEQPI

SEQ ID NO: 691 - protein, 9 aa, Artificial Sequence
  Synthetic Polypeptide
PQPEQPIPV

SEQ ID NO: 692 - protein, 9 aa, Artificial Sequence
  Synthetic Polypeptide
EQPIPVQPE

SEQ ID NO: 693 - protein, 9 aa, Artificial Sequence
  Synthetic Polypeptide
PFPQPEQPT

SEQ ID NO: 694 - protein, 9 aa, Artificial Sequence
  Synthetic Polypeptide
PQPEQPTPI

SEQ ID NO: 695 - protein, 9 aa, Artificial Sequence
  Synthetic Polypeptide
EQPTPIQPE

SEQ ID NO: 696 - protein, 9 aa, Artificial Sequence
  Synthetic Polypeptide
PFPQPEQPF

SEQ ID NO: 697 - protein, 9 aa, Artificial Sequence
  Synthetic Polypeptide
PQPEQPFPL

SEQ ID NO: 698 - protein, 9 aa, Artificial Sequence
  Synthetic Polypeptide
EQPFPLQPE

SEQ ID NO: 699 - protein, 9 aa, Artificial Sequence
  Synthetic Polypeptide
PFPQPEQPF

SEQ ID NO: 700 - protein, 9 aa, Artificial Sequence
  Synthetic Polypeptide
PQPEQPFSQ

SEQ ID NO: 701 - protein, 9 aa, Artificial Sequence
  Synthetic Polypeptide
PYPEQPQPF

SEQ ID NO: 702 - protein, 9 aa, Artificial Sequence
  Synthetic Polypeptide
PFPEQPEQI

SEQ ID NO: 703 - protein, 9 aa, Artificial Sequence
  Synthetic Polypeptide
EGSFQPSQE

SEQ ID NO: 704 - protein, 9 aa, Artificial Sequence
  Synthetic Polypeptide
QGYYPTSPQ

SEQ ID NO: 705 - protein, 9 aa, Artificial Sequence
  Synthetic Polypeptide
EQPEQPFPE

SEQ ID NO: 706 - protein, 9 aa, Artificial Sequence
  Synthetic Polypeptide
EQPFPEQPQ

SEQ ID NO: 707 - protein, 9 aa, Artificial Sequence
  Synthetic Polypeptide
PFSEQEQPV

SEQ ID NO: 708 - protein, 9 aa, Artificial Sequence
  Synthetic Polypeptide
EQPFPEQPI

SEQ ID NO: 709 - protein, 9 aa, Artificial Sequence
  Synthetic Polypeptide
PFPEQPIPE

SEQ ID NO: 710 - protein, 9 aa, Artificial Sequence
  Synthetic Polypeptide
EQPIPEQPQ

SEQ ID NO: 711 - protein, 9 aa, Artificial Sequence
  Synthetic Polypeptide
PIPEQPQPY

SEQ ID NO: 712 - protein, 9 aa, Artificial Sequence
  Synthetic Polypeptide
PQPELPYPQ

SEQ ID NO: 713 - protein, 9 aa, Artificial Sequence
  Synthetic Polypeptide
PYPQPELPY

SEQ ID NO: 714 - protein, 9 aa, Artificial Sequence
  Synthetic Polypeptide
PFPQPELPY

SEQ ID NO: 715 - protein, 9 aa, Artificial Sequence
  Synthetic Polypeptide
PQPELPYPY

SEQ ID NO: 716 - protein, 9 aa, Artificial Sequence
  Synthetic Polypeptide
EQPFPEQPI

SEQ ID NO: 717 - protein, 9 aa, Artificial Sequence
  Synthetic Polypeptide
PFPEQPIPE

SEQ ID NO: 718 - protein, 9 aa, Artificial Sequence
  Synthetic Polypeptide
PQPYPEQPQ

SEQ ID NO: 719 - protein, 9 aa, Artificial Sequence
  Synthetic Polypeptide
PYPEQPQPF

SEQ ID NO: 720 - protein, 15 aa, Artificial Sequence
  Synthetic Polypeptide
LQPFPQPELPYPQPQ

SEQ ID NO: 721 - protein, 14 aa, Artificial Sequence
  Synthetic Polypeptide
QPFPQPEQPFPWQP

SEQ ID NO: 722 - protein, 15 aa, Artificial Sequence
  Synthetic Polypeptide
PEQPIPEQPQPYPQQ

SEQ ID NO: 723 - protein, 17 aa, Artificial Sequence
  Synthetic Polypeptide
QPFPQPEQPIPVQPEQS

SEQ ID NO: 724 - protein, 17 aa, Artificial Sequence
  Synthetic Polypeptide
QPFPQPEQPTPIQPEQP

SEQ ID NO: 725 - protein, 17 aa, Artificial Sequence
  Synthetic Polypeptide
QPFPQPEQPFPLQPEQP

SEQ ID NO: 726 - protein, 13 aa, Artificial Sequence
  Synthetic Polypeptide
QPFPQPEQPFSQQ

SEQ ID NO: 727 - protein, 14 aa, Artificial Sequence
  Synthetic Polypeptide
PQPYPEQPQPFPQQ

SEQ ID NO: 728 - protein, 15 aa, Artificial Sequence
  Synthetic Polypeptide
QPFPEQPEQIIPQQP

SEQ ID NO: 729 - protein, 14 aa, Artificial Sequence
  Synthetic Polypeptide
SGEGSFQPSQENPQ

SEQ ID NO: 730 - protein, 14 aa, Artificial Sequence
  Synthetic Polypeptide
GQQGYYPTSPQQSG

SEQ ID NO: 731 - protein, 14 aa, Artificial Sequence
  Synthetic Polypeptide
PEQPEQPFPEQPQQ

SEQ ID NO: 732 - protein, 14 aa, Artificial Sequence
  Synthetic Polypeptide
QPPFSEQEQPVLPQ

SEQ ID NO: 733 - protein, 18 aa, Artificial Sequence
  Synthetic Polypeptide
PEQPFPEQPIPEQPQPYP

SEQ ID NO: 734 - protein, 14 aa, Artificial Sequence
  Synthetic Polypeptide
QPYPQPELPYPQPQ

SEQ ID NO: 735 - protein, 14 aa, Artificial Sequence
  Synthetic Polypeptide
QPFPQPELPYPYPQ

SEQ ID NO: 736 - protein, 15 aa, Artificial Sequence
  Synthetic Polypeptide
PQEQPFPEQPIPEQP

SEQ ID NO: 737 - protein, 15 aa, Artificial Sequence
  Synthetic Polypeptide
QPQPYPEQPQPFPQQ

SEQ ID NO: 738 - protein, 9 aa, Artificial Sequence
  Synthetic Polypeptide
PFPQPELPY

SEQ ID NO: 739 - protein, 9 aa, Artificial Sequence
  Synthetic Polypeptide
PQPELPYPQ

SEQ ID NO: 740 - protein, 9 aa, Artificial Sequence
  Synthetic Polypeptide
PFPQPEQPF

SEQ ID NO: 741 - protein, 9 aa, Artificial Sequence
  Synthetic Polypeptide
PQPEQPFPW

SEQ ID NO: 742 - protein, 9 aa, Artificial Sequence
  Synthetic Polypeptide
EQPIPEQPQ

SEQ ID NO: 743 - protein, 9 aa, Artificial Sequence
  Synthetic Polypeptide
PIPEQPQPY

SEQ ID NO: 744 - protein, 9 aa, Artificial Sequence
  Synthetic Polypeptide
PFPQPEQPI

SEQ ID NO: 745 - protein, 9 aa, Artificial Sequence
  Synthetic Polypeptide
PQPEQPIPV

SEQ ID NO: 746 - protein, 9 aa, Artificial Sequence
  Synthetic Polypeptide
EQPIPVQPE

SEQ ID NO: 747 - protein, 9 aa, Artificial Sequence
  Synthetic Polypeptide
PFPQPEQPT

SEQ ID NO: 748 - protein, 9 aa, Artificial Sequence
  Synthetic Polypeptide
PQPEQPTPI

SEQ ID NO: 749 - protein, 9 aa, Artificial Sequence
  Synthetic Polypeptide
EQPTPIQPE

SEQ ID NO: 750 - protein, 9 aa, Artificial Sequence
  Synthetic Polypeptide
PQPEQPFPL

SEQ ID NO: 751 - protein, 9 aa, Artificial Sequence
  Synthetic Polypeptide
EQPFPLQPE

SEQ ID NO: 752 - protein, 9 aa, Artificial Sequence
  Synthetic Polypeptide
EGSFQPSQE

SEQ ID NO: 753 - protein, 9 aa, Artificial Sequence
  Synthetic Polypeptide
QGYYPTSPQ

SEQ ID NO: 754 - protein, 9 aa, Artificial Sequence
  Synthetic Polypeptide
EQPEQPFPE

SEQ ID NO: 755 - protein, 9 aa, Artificial Sequence
  Synthetic Polypeptide
PFSEQEQPV

SEQ ID NO: 756 - protein, 9 aa, Artificial Sequence
  Synthetic Polypeptide
PYPQPELPY

SEQ ID NO: 757 - protein, 9 aa, Artificial Sequence
  Synthetic Polypeptide
EQPFPEQPI

SEQ ID NO: 758 - protein, 9 aa, Artificial Sequence
  Synthetic Polypeptide
PFPEQPIPE

SEQ ID NO: 759 - protein, 9 aa, Artificial Sequence
  Synthetic Polypeptide
PYPEQPQPF

SEQ ID NO: 760 - protein, 9 aa, Artificial Sequence
  Synthetic Polypeptide
PQPYPEQPQ

SEQ ID NO: 761 - protein, 9 aa, Artificial Sequence
  Synthetic Polypeptide
PFPQPELPY

SEQ ID NO: 762 - protein, 9 aa, Artificial Sequence
  Synthetic Polypeptide
PQPELPYPQ

SEQ ID NO: 763 - protein, 9 aa, Artificial Sequence
  Synthetic Polypeptide
PFPQPEQPF

SEQ ID NO: 764 - protein, 9 aa, Artificial Sequence
  Synthetic Polypeptide
PQPEQPFPW

SEQ ID NO: 765 - protein, 9 aa, Artificial Sequence
  Synthetic Polypeptide
EQPIPEQPQ

SEQ ID NO: 766 - protein, 9 aa, Artificial Sequence
  Synthetic Polypeptide
PIPEQPQPY

SEQ ID NO: 767 - protein, 9 aa, Artificial Sequence
  Synthetic Polypeptide
PFPQPEQPI

SEQ ID NO: 768 - protein, 9 aa, Artificial Sequence
  Synthetic Polypeptide
PQPEQPIPV

SEQ ID NO: 769 - protein, 9 aa, Artificial Sequence
  Synthetic Polypeptide
EQPIPVQPE

SEQ ID NO: 770 - protein, 9 aa, Artificial Sequence
  Synthetic Polypeptide
PFPQPEQPT

SEQ ID NO: 771 - protein, 9 aa, Artificial Sequence
  Synthetic Polypeptide
PQPEQPTPI

SEQ ID NO: 772 - protein, 9 aa, Artificial Sequence
  Synthetic Polypeptide
EQPTPIQPE

SEQ ID NO: 773 - protein, 9 aa, Artificial Sequence
  Synthetic Polypeptide
PQPEQPFPL

SEQ ID NO: 774 - protein, 9 aa, Artificial Sequence
  Synthetic Polypeptide
EQPFPLQPE

SEQ ID NO: 775 - protein, 9 aa, Artificial Sequence
  Synthetic Polypeptide
EGSFQPSQE

SEQ ID NO: 776 - protein, 9 aa, Artificial Sequence
  Synthetic Polypeptide
QGYYPTSPQ

SEQ ID NO: 777 - protein, 9 aa, Artificial Sequence
  Synthetic Polypeptide
EQPEQPFPE

SEQ ID NO: 778 - protein, 9 aa, Artificial Sequence
  Synthetic Polypeptide
PFSEQEQPV

SEQ ID NO: 779 - protein, 9 aa, Artificial Sequence
  Synthetic Polypeptide
PYPQPELPY

SEQ ID NO: 780 - protein, 9 aa, Artificial Sequence
  Synthetic Polypeptide
EQPFPEQPI

SEQ ID NO: 781 - protein, 9 aa, Artificial Sequence
  Synthetic Polypeptide
PFPEQPIPE

SEQ ID NO: 782 - protein, 9 aa, Artificial Sequence
  Synthetic Polypeptide
PYPEQPQPF

SEQ ID NO: 783 - protein, 9 aa, Artificial Sequence
  Synthetic Polypeptide
PQPYPEQPQ

SEQ ID NO: 784 - protein, 9 aa, Artificial Sequence
  Synthetic Polypeptide
PFPQPELPY

SEQ ID NO: 785 - protein, 9 aa, Artificial Sequence
  Synthetic Polypeptide
PQPELPYPQ

SEQ ID NO: 786 - protein, 9 aa, Artificial Sequence
  Synthetic Polypeptide
PFPQPEQPF

SEQ ID NO: 787 - protein, 9 aa, Artificial Sequence
  Synthetic Polypeptide
PQPEQPFPW

SEQ ID NO: 788 - protein, 9 aa, Artificial Sequence
  Synthetic Polypeptide
EQPIPEQPQ

SEQ ID NO: 789 - protein, 9 aa, Artificial Sequence
  Synthetic Polypeptide
PIPEQPQPY

SEQ ID NO: 790 - protein, 9 aa, Artificial Sequence
  Synthetic Polypeptide
PFPQPEQPI

SEQ ID NO: 791 - protein, 9 aa, Artificial Sequence
  Synthetic Polypeptide
PQPEQPIPV

SEQ ID NO: 792 - protein, 9 aa, Artificial Sequence
  Synthetic Polypeptide
EQPIPVQPE

SEQ ID NO: 793 - protein, 9 aa, Artificial Sequence
  Synthetic Polypeptide
PFPQPEQPT

SEQ ID NO: 794 - protein, 9 aa, Artificial Sequence
  Synthetic Polypeptide
PQPEQPTPI

SEQ ID NO: 795 - protein, 9 aa, Artificial Sequence
  Synthetic Polypeptide
EQPTPIQPE

SEQ ID NO: 796 - protein, 9 aa, Artificial Sequence
  Synthetic Polypeptide
PQPEQPFPL

SEQ ID NO: 797 - protein, 9 aa, Artificial Sequence
  Synthetic Polypeptide
EQPFPLQPE

SEQ ID NO: 798 - protein, 9 aa, Artificial Sequence
  Synthetic Polypeptide
EGSFQPSQE

SEQ ID NO: 799 - protein, 9 aa, Artificial Sequence
  Synthetic Polypeptide
QGYYPTSPQ

SEQ ID NO: 800 - protein, 9 aa, Artificial Sequence
  Synthetic Polypeptide
EQPEQPFPE

SEQ ID NO: 801 - protein, 9 aa, Artificial Sequence
  Synthetic Polypeptide
PFSEQEQPV

SEQ ID NO: 802 - protein, 9 aa, Artificial Sequence
  Synthetic Polypeptide
PYPQPELPY

SEQ ID NO: 803 - protein, 9 aa, Artificial Sequence
  Synthetic Polypeptide
EQPFPEQPI

SEQ ID NO: 804 - protein, 9 aa, Artificial Sequence
  Synthetic Polypeptide
PFPEQPIPE

SEQ ID NO: 805 - protein, 9 aa, Artificial Sequence
  Synthetic Polypeptide
PYPEQPQPF

SEQ ID NO: 806 - protein, 9 aa, Artificial Sequence
  Synthetic Polypeptide
PQPYPEQPQ

SEQ ID NO: 807 - protein, 12 aa, Artificial Sequence
  Synthetic Polypeptide
PFPQPELPYPQP

SEQ ID NO: 808 - protein, 12 aa, Artificial Sequence
  Synthetic Polypeptide
PFPQPEQPFPWQ

SEQ ID NO: 809 - protein, 12 aa, Artificial Sequence
  Synthetic Polypeptide
EQPIPEQPQPYP

SEQ ID NO: 810 - protein, 12 aa, Artificial Sequence
  Synthetic Polypeptide
PFPQPEQPIPVQ

SEQ ID NO: 811 - protein, 12 aa, Artificial Sequence
  Synthetic Polypeptide
PEQPIPVQPEQS

SEQ ID NO: 812 - protein, 12 aa, Artificial Sequence
  Synthetic Polypeptide
PFPQPEQPTPIQ

SEQ ID NO: 813 - protein, 12 aa, Artificial Sequence
  Synthetic Polypeptide
PEQPTPIQPEQP

SEQ ID NO: 814 - protein, 12 aa, Artificial Sequence
  Synthetic Polypeptide
PFPQPEQPFPLQ

SEQ ID NO: 815 - protein, 12 aa, Artificial Sequence
  Synthetic Polypeptide
PEQPFPLQPEQP

SEQ ID NO: 816 - protein, 12 aa, Artificial Sequence
  Synthetic Polypeptide
GEGSFQPSQENP

SEQ ID NO: 817 - protein, 12 aa, Artificial Sequence
  Synthetic Polypeptide
QQGYYPTSPQQS

SEQ ID NO: 818 - protein, 12 aa, Artificial Sequence
  Synthetic Polypeptide
PEQPEQPFPEQP

SEQ ID NO: 819 - protein, 12 aa, Artificial Sequence
  Synthetic Polypeptide
PPFSEQEQPVLP

SEQ ID NO: 820 - protein, 12 aa, Artificial Sequence
  Synthetic Polypeptide
PYPQPELPYPQP

SEQ ID NO: 821 - protein, 12 aa, Artificial Sequence
  Synthetic Polypeptide
EQPFPEQPIPEQ

SEQ ID NO: 822 - protein, 12 aa, Artificial Sequence
  Synthetic Polypeptide
PQPYPEQPQPFP

SEQ ID NO: 823 - protein, 9 aa, Artificial Sequence
  Synthetic Polypeptide
PFPQPELPY

SEQ ID NO: 824 - protein, 9 aa, Artificial Sequence
  Synthetic Polypeptide
PQPELPYPQ

SEQ ID NO: 825 - protein, 9 aa, Artificial Sequence
  Synthetic Polypeptide
PFPQPEQPF

SEQ ID NO: 826 - protein, 9 aa, Artificial Sequence
  Synthetic Polypeptide
PQPEQPFPW

SEQ ID NO: 827 - protein, 9 aa, Artificial Sequence
  Synthetic Polypeptide
PIPEQPQPY

SEQ ID NO: 828 - protein, 10 aa, Artificial Sequence
  Synthetic Polypeptide
PFPQPEQPIP

SEQ ID NO: 829 - protein, 9 aa, Artificial Sequence
  Synthetic Polypeptide
EQPIPVQPE

SEQ ID NO: 830 - protein, 11 aa, Artificial Sequence
  Synthetic Polypeptide
PFPQPEQPTPI

SEQ ID NO: 831 - protein, 9 aa, Artificial Sequence
  Synthetic Polypeptide
EQPTPIQPE

SEQ ID NO: 832 - protein, 9 aa, Artificial Sequence
  Synthetic Polypeptide
PQPEQPFPL

SEQ ID NO: 833 - protein, 9 aa, Artificial Sequence
  Synthetic Polypeptide
EQPFPLQPE

SEQ ID NO: 834 - protein, 9 aa, Artificial Sequence
  Synthetic Polypeptide
PFPQPEQPF

SEQ ID NO: 835 - protein, 9 aa, Artificial Sequence
  Synthetic Polypeptide
PQPEQPFSQ

SEQ ID NO: 836 - protein, 9 aa, Artificial Sequence
  Synthetic Polypeptide
PYPEQPQPF

SEQ ID NO: 837 - protein, 11 aa, Artificial Sequence
  Synthetic Polypeptide
PFPEQPEQIIP

SEQ ID NO: 838 - protein, 9 aa, Artificial Sequence
  Synthetic Polypeptide
EGSFQPSQE

SEQ ID NO: 839 - protein, 9 aa, Artificial Sequence
  Synthetic Polypeptide
QGYYPTSPQ

SEQ ID NO: 840 - protein, 12 aa, Artificial Sequence
  Synthetic Polypeptide
EQPEQPFPEQPQ

SEQ ID NO: 841 - protein, 9 aa, Artificial Sequence
  Synthetic Polypeptide
PFSEQEQPV

SEQ ID NO: 842 - protein, 9 aa, Artificial Sequence
  Synthetic Polypeptide
EQPFPEQPI

SEQ ID NO: 843 - protein, 9 aa, Artificial Sequence
  Synthetic Polypeptide
PIPEQPQPY

SEQ ID NO: 844 - protein, 9 aa, Artificial Sequence
  Synthetic Polypeptide
PQPELPYPQ

SEQ ID NO: 845 - protein, 9 aa, Artificial Sequence
  Synthetic Polypeptide
PYPQPELPY

SEQ ID NO: 846 - protein, 9 aa, Artificial Sequence
  Synthetic Polypeptide
PFPQPELPY

SEQ ID NO: 847 - protein, 9 aa, Artificial Sequence
  Synthetic Polypeptide
PQPELPYPY

SEQ ID NO: 848 - protein, 9 aa, Artificial Sequence
  Synthetic Polypeptide
EQPFPEQPI

SEQ ID NO: 849 - protein, 15 aa, Artificial Sequence
  Synthetic Polypeptide
LQPFPQPELPYPQPQ

SEQ ID NO: 850 - protein, 14 aa, Artificial Sequence
  Synthetic Polypeptide
QPFPQPEQPFPWQP

SEQ ID NO: 851 - protein, 15 aa, Artificial Sequence
  Synthetic Polypeptide
PEQPIPEQPQPYPQQ

SEQ ID NO: 852 - protein, 17 aa, Artificial Sequence
  Synthetic Polypeptide
QPFPQPEQPIPVQPEQS

SEQ ID NO: 853 - protein, 17 aa, Artificial Sequence
  Synthetic Polypeptide
QPFPQPEQPTPIQPEQP

SEQ ID NO: 854 - protein, 17 aa, Artificial Sequence
  Synthetic Polypeptide
QPFPQPEQPFPLQPEQP

SEQ ID NO: 855 - protein, 13 aa, Artificial Sequence
  Synthetic Polypeptide
QPFPQPEQPFSQQ

SEQ ID NO: 856 - protein, 14 aa, Artificial Sequence
  Synthetic Polypeptide
PQPYPEQPQPFPQQ

SEQ ID NO: 857 - protein, 15 aa, Artificial Sequence
  Synthetic Polypeptide
QPFPEQPEQIIPQQP

SEQ ID NO: 858 - protein, 14 aa, Artificial Sequence
  Synthetic Polypeptide
SGEGSFQPSQENPQ

SEQ ID NO: 859 - protein, 14 aa, Artificial Sequence
  Synthetic Polypeptide
GQQGYYPTSPQQSG

SEQ ID NO: 860 - protein, 14 aa, Artificial Sequence
  Synthetic Polypeptide
PEQPEQPFPEQPQQ

SEQ ID NO: 861 - protein, 14 aa, Artificial Sequence
  Synthetic Polypeptide
QPPFSEQEQPVLPQ

SEQ ID NO: 862 - protein, 18 aa, Artificial Sequence
  Synthetic Polypeptide
PEQPFPEQPIPEQPQPYP

SEQ ID NO: 863 - protein, 14 aa, Artificial Sequence
  Synthetic Polypeptide
QPYPQPELPYPQPQ

SEQ ID NO: 864 - protein, 14 aa, Artificial Sequence
  Synthetic Polypeptide
QPFPQPELPYPYPQ

SEQ ID NO: 865 - protein, 9 aa, Artificial Sequence
  Synthetic Polypeptide
EQPFPEQPI

SEQ ID NO: 866 - DNA, 1227 bp, Homo sapiens
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 867 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 868 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 869 - protein, 9 aa, Artificial Sequence
  Synthetic Polypeptide
PFPQPELPY

SEQ ID NO: 870 - protein, 9 aa, Artificial Sequence
  Synthetic Polypeptide
PQPELPYPQ

SEQ ID NO: 871 - protein, 9 aa, Artificial Sequence
  Synthetic Polypeptide
PFPQPEQPF

SEQ ID NO: 872 - protein, 9 aa, Artificial Sequence
  Synthetic Polypeptide
PQPEQPFPW

SEQ ID NO: 873 - protein, 9 aa, Artificial Sequence
  Synthetic Polypeptide
PIPEQPQPY

SEQ ID NO: 874 - protein, 15 aa, Artificial Sequence
  Synthetic Polypeptide
LQPFPQPELPYPQPQ

SEQ ID NO: 875 - protein, 14 aa, Artificial Sequence
  Synthetic Polypeptide
QPFPQPEQPFPWQP

SEQ ID NO: 876 - protein, 15 aa, Artificial Sequence
  Synthetic Polypeptide
PEQPIPEQPQPYPQQ

SEQ ID NO: 877 - protein, 16 aa, Artificial Sequence
  Synthetic Polypeptide
ELQPFPQPELPYPQPQ

SEQ ID NO: 878 - protein, 15 aa, Artificial Sequence
  Synthetic Polypeptide
EQPFPQPEQPFPWQP

SEQ ID NO: 879 - protein, 16 aa, Artificial Sequence
  Synthetic Polypeptide
EPEQPIPEQPQPYPQQ

SEQ ID NO: 880 - protein, 16 aa, Artificial Sequence
  Synthetic Polypeptide
ELQPFPQPELPYPQPQ

SEQ ID NO: 881 - protein, 15 aa, Artificial Sequence
  Synthetic Polypeptide
EQPFPQPEQPFPWQP

SEQ ID NO: 882 - protein, 16 aa, Artificial Sequence
  Synthetic Polypeptide
EPEQPIPEQPQPYPQQ

SEQ ID NO: 883 - protein, 9 aa, Artificial Sequence
  Synthetic Polypeptide
PFPQPELPY

SEQ ID NO: 884 - protein, 9 aa, Artificial Sequence
  Synthetic Polypeptide
PQPELPYPQ

SEQ ID NO: 885 - protein, 9 aa, Artificial Sequence
  Synthetic Polypeptide
PFPQPEQPF

SEQ ID NO: 886 - protein, 9 aa, Artificial Sequence
  Synthetic Polypeptide
PQPEQPFPW

SEQ ID NO: 887 - protein, 9 aa, Artificial Sequence
  Synthetic Polypeptide
EQPIPEQPQ

SEQ ID NO: 888 - protein, 9 aa, Artificial Sequence
  Synthetic Polypeptide
PIPEQPQPY

SEQ ID NO: 889 - protein, 9 aa, Artificial Sequence
  Synthetic Polypeptide
PFPQPEQPI

SEQ ID NO: 890 - protein, 9 aa, Artificial Sequence
  Synthetic Polypeptide
PQPEQPIPV

SEQ ID NO: 891 - protein, 9 aa, Artificial Sequence
  Synthetic Polypeptide
EQPIPVQPE

SEQ ID NO: 892 - protein, 9 aa, Artificial Sequence
  Synthetic Polypeptide
PFPQPEQPT

SEQ ID NO: 893 - protein, 9 aa, Artificial Sequence
  Synthetic Polypeptide
PQPEQPTPI

SEQ ID NO: 894 - protein, 9 aa, Artificial Sequence
  Synthetic Polypeptide
EQPTPIQPE

SEQ ID NO: 895 - protein, 9 aa, Artificial Sequence
  Synthetic Polypeptide
PFPQPEQPF

SEQ ID NO: 896 - protein, 9 aa, Artificial Sequence
  Synthetic Polypeptide
PQPEQPFPL

SEQ ID NO: 897 - protein, 9 aa, Artificial Sequence
  Synthetic Polypeptide
EQPFPLQPE

SEQ ID NO: 898 - protein, 9 aa, Artificial Sequence
  Synthetic Polypeptide
PFPQPEQPF

SEQ ID NO: 899 - protein, 9 aa, Artificial Sequence
  Synthetic Polypeptide
PQPEQPFSQ

SEQ ID NO: 900 - protein, 9 aa, Artificial Sequence
  Synthetic Polypeptide
PYPEQPQPF

SEQ ID NO: 901 - protein, 9 aa, Artificial Sequence
  Synthetic Polypeptide
PFPEQPEQI

SEQ ID NO: 902 - protein, 9 aa, Artificial Sequence
  Synthetic Polypeptide
EGSFQPSQE

SEQ ID NO: 903 - protein, 9 aa, Artificial Sequence
  Synthetic Polypeptide
QGYYPTSPQ

SEQ ID NO: 904 - protein, 9 aa, Artificial Sequence
  Synthetic Polypeptide
EQPEQPFPE

SEQ ID NO: 905 - protein, 9 aa, Artificial Sequence
  Synthetic Polypeptide
EQPFPEQPQ

SEQ ID NO: 906 - protein, 9 aa, Artificial Sequence
  Synthetic Polypeptide
PFSEQEQPV

SEQ ID NO: 907 - protein, 9 aa, Artificial Sequence
  Synthetic Polypeptide
EQPFPEQPI

SEQ ID NO: 908 - protein, 9 aa, Artificial Sequence
  Synthetic Polypeptide
PFPEQPIPE

SEQ ID NO: 909 - protein, 9 aa, Artificial Sequence
  Synthetic Polypeptide
EQPIPEQPQ

SEQ ID NO: 910 - protein, 9 aa, Artificial Sequence
  Synthetic Polypeptide
PIPEQPQPY

SEQ ID NO: 911 - protein, 9 aa, Artificial Sequence
  Synthetic Polypeptide
PQPELPYPQ

SEQ ID NO: 912 - protein, 9 aa, Artificial Sequence
  Synthetic Polypeptide
PYPQPELPY

SEQ ID NO: 913 - protein, 9 aa, Artificial Sequence
  Synthetic Polypeptide
PFPQPELPY

SEQ ID NO: 914 - protein, 9 aa, Artificial Sequence
  Synthetic Polypeptide
PQPELPYPY

SEQ ID NO: 915 - protein, 9 aa, Artificial Sequence
  Synthetic Polypeptide
EQPFPEQPI

SEQ ID NO: 916 - protein, 9 aa, Artificial Sequence
  Synthetic Polypeptide
PFPEQPIPE

SEQ ID NO: 917 - protein, 9 aa, Artificial Sequence
  Synthetic Polypeptide
PQPYPEQPQ

SEQ ID NO: 918 - protein, 9 aa, Artificial Sequence
  Synthetic Polypeptide
PYPEQPQPF

SEQ ID NO: 919 - protein, 15 aa, Artificial Sequence
  Synthetic Polypeptide
LQPFPQPELPYPQPQ

SEQ ID NO: 920 - protein, 14 aa, Artificial Sequence
  Synthetic Polypeptide
QPFPQPEQPFPWQP

SEQ ID NO: 921 - protein, 15 aa, Artificial Sequence
  Synthetic Polypeptide
PEQPIPEQPQPYPQQ

SEQ ID NO: 922 - protein, 17 aa, Artificial Sequence
  Synthetic Polypeptide
QPFPQPEQPIPVQPEQS

SEQ ID NO: 923 - protein, 17 aa, Artificial Sequence
  Synthetic Polypeptide
QPFPQPEQPTPIQPEQP

SEQ ID NO: 924 - protein, 17 aa, Artificial Sequence
  Synthetic Polypeptide
QPFPQPEQPFPLQPEQP

SEQ ID NO: 925 - protein, 13 aa, Artificial Sequence
  Synthetic Polypeptide
QPFPQPEQPFSQQ

SEQ ID NO: 926 - protein, 14 aa, Artificial Sequence
  Synthetic Polypeptide
PQPYPEQPQPFPQQ

SEQ ID NO: 927 - protein, 15 aa, Artificial Sequence
  Synthetic Polypeptide
QPFPEQPEQIIPQQP

SEQ ID NO: 928 - protein, 14 aa, Artificial Sequence
  Synthetic Polypeptide
SGEGSFQPSQENPQ

SEQ ID NO: 929 - protein, 14 aa, Artificial Sequence
  Synthetic Polypeptide
GQQGYYPTSPQQSG

SEQ ID NO: 930 - protein, 14 aa, Artificial Sequence
  Synthetic Polypeptide
PEQPEQPFPEQPQQ

SEQ ID NO: 931 - protein, 14 aa, Artificial Sequence
  Synthetic Polypeptide
QPPFSEQEQPVLPQ

SEQ ID NO: 932 - protein, 18 aa, Artificial Sequence
  Synthetic Polypeptide
PEQPFPEQPIPEQPQPYP

SEQ ID NO: 933 - protein, 14 aa, Artificial Sequence
  Synthetic Polypeptide
QPYPQPELPYPQPQ

SEQ ID NO: 934 - protein, 14 aa, Artificial Sequence
  Synthetic Polypeptide
QPFPQPELPYPYPQ

SEQ ID NO: 935 - protein, 15 aa, Artificial Sequence
  Synthetic Polypeptide
PQEQPFPEQPIPEQP

SEQ ID NO: 936 - protein, 15 aa, Artificial Sequence
  Synthetic Polypeptide
QPQPYPEQPQPFPQQ

SEQ ID NO: 937 - protein, 9 aa, Artificial Sequence
  Synthetic Polypeptide
PFPQPELPY

SEQ ID NO: 938 - protein, 9 aa, Artificial Sequence
  Synthetic Polypeptide
PQPELPYPQ

SEQ ID NO: 939 - protein, 9 aa, Artificial Sequence
  Synthetic Polypeptide
PFPQPEQPF

SEQ ID NO: 940 - protein, 9 aa, Artificial Sequence
  Synthetic Polypeptide
PQPEQPFPW

SEQ ID NO: 941 - protein, 9 aa, Artificial Sequence
  Synthetic Polypeptide
PIPEQPQPY

SEQ ID NO: 942 - protein, 10 aa, Artificial Sequence
  Synthetic Polypeptide
PFPQPEQPIP

SEQ ID NO: 943 - protein, 9 aa, Artificial Sequence
  Synthetic Polypeptide
EQPIPVQPE

SEQ ID NO: 944 - protein, 11 aa, Artificial Sequence
  Synthetic Polypeptide
PFPQPEQPTPI

SEQ ID NO: 945 - protein, 9 aa, Artificial Sequence
  Synthetic Polypeptide
EQPTPIQPE

SEQ ID NO: 946 - protein, 9 aa, Artificial Sequence
  Synthetic Polypeptide
PQPEQPFPL

SEQ ID NO: 947 - protein, 9 aa, Artificial Sequence
  Synthetic Polypeptide
EQPFPLQPE

SEQ ID NO: 948 - protein, 9 aa, Artificial Sequence
  Synthetic Polypeptide
PFPQPEQPF

SEQ ID NO: 949 - protein, 9 aa, Artificial Sequence
  Synthetic Polypeptide
PQPEQPFSQ

SEQ ID NO: 950 - protein, 9 aa, Artificial Sequence
  Synthetic Polypeptide
PYPEQPQPF

SEQ ID NO: 951 - protein, 11 aa, Artificial Sequence
  Synthetic Polypeptide
PFPEQPEQIIP

SEQ ID NO: 952 - protein, 9 aa, Artificial Sequence
  Synthetic Polypeptide
EGSFQPSQE

SEQ ID NO: 953 - protein, 9 aa, Artificial Sequence
  Synthetic Polypeptide
QGYYPTSPQ

SEQ ID NO: 954 - protein, 12 aa, Artificial Sequence
  Synthetic Polypeptide
EQPEQPFPEQPQ

SEQ ID NO: 955 - protein, 9 aa, Artificial Sequence
  Synthetic Polypeptide
PFSEQEQPV

SEQ ID NO: 956 - protein, 9 aa, Artificial Sequence
  Synthetic Polypeptide
EQPFPEQPI

SEQ ID NO: 957 - protein, 9 aa, Artificial Sequence
  Synthetic Polypeptide
PIPEQPQPY

SEQ ID NO: 958 - protein, 9 aa, Artificial Sequence
  Synthetic Polypeptide
PQPELPYPQ

SEQ ID NO: 959 - protein, 9 aa, Artificial Sequence
  Synthetic Polypeptide
PYPQPELPY

SEQ ID NO: 960 - protein, 9 aa, Artificial Sequence
  Synthetic Polypeptide
PFPQPELPY

SEQ ID NO: 961 - protein, 9 aa, Artificial Sequence
  Synthetic Polypeptide
PQPELPYPY

SEQ ID NO: 962 - protein, 9 aa, Artificial Sequence
  Synthetic Polypeptide
EQPFPEQPI

SEQ ID NO: 963 - protein, 15 aa, Artificial Sequence
  Synthetic Polypeptide
LQPFPQPELPYPQPQ

SEQ ID NO: 964 - protein, 14 aa, Artificial Sequence
  Synthetic Polypeptide
QPFPQPEQPFPWQP

SEQ ID NO: 965 - protein, 15 aa, Artificial Sequence
  Synthetic Polypeptide
PEQPIPEQPQPYPQQ

SEQ ID NO: 966 - protein, 17 aa, Artificial Sequence
  Synthetic Polypeptide
QPFPQPEQPIPVQPEQS

SEQ ID NO: 967 - protein, 17 aa, Artificial Sequence
  Synthetic Polypeptide
QPFPQPEQPTPIQPEQP

SEQ ID NO: 968 - protein, 17 aa, Artificial Sequence
  Synthetic Polypeptide
QPFPQPEQPFPLQPEQP

SEQ ID NO: 969 - protein, 13 aa, Artificial Sequence
  Synthetic Polypeptide
QPFPQPEQPFSQQ

SEQ ID NO: 970 - protein, 14 aa, Artificial Sequence
  Synthetic Polypeptide
PQPYPEQPQPFPQQ

SEQ ID NO: 971 - protein, 15 aa, Artificial Sequence
  Synthetic Polypeptide
QPFPEQPEQIIPQQP

SEQ ID NO: 972 - protein, 14 aa, Artificial Sequence
  Synthetic Polypeptide
SGEGSFQPSQENPQ

SEQ ID NO: 973 - protein, 14 aa, Artificial Sequence
  Synthetic Polypeptide
GQQGYYPTSPQQSG

SEQ ID NO: 974 - protein, 14 aa, Artificial Sequence
  Synthetic Polypeptide
PEQPEQPFPEQPQQ

SEQ ID NO: 975 - protein, 14 aa, Artificial Sequence
  Synthetic Polypeptide
QPPFSEQEQPVLPQ

SEQ ID NO: 976 - protein, 18 aa, Artificial Sequence
  Synthetic Polypeptide
PEQPFPEQPIPEQPQPYP

SEQ ID NO: 977 - protein, 14 aa, Artificial Sequence
  Synthetic Polypeptide
QPYPQPELPYPQPQ

SEQ ID NO: 978 - protein, 14 aa, Artificial Sequence
  Synthetic Polypeptide
QPFPQPELPYPYPQ

SEQ ID NO: 979 - protein, 9 aa, Artificial Sequence
  Synthetic Polypeptide
EQPFPEQPI

SEQ ID NO: 980 - protein, 9 aa, Artificial Sequence
  Synthetic Polypeptide
PFPQPELPY

SEQ ID NO: 981 - protein, 9 aa, Artificial Sequence
  Synthetic Polypeptide
PQPELPYPQ

SEQ ID NO: 982 - protein, 9 aa, Artificial Sequence
  Synthetic Polypeptide
PFPQPEQPF

SEQ ID NO: 983 - protein, 9 aa, Artificial Sequence
  Synthetic Polypeptide
PQPEQPFPW

SEQ ID NO: 984 - protein, 9 aa, Artificial Sequence
  Synthetic Polypeptide
EQPIPEQPQ

SEQ ID NO: 985 - protein, 9 aa, Artificial Sequence
  Synthetic Polypeptide
PIPEQPQPY

SEQ ID NO: 986 - protein, 9 aa, Artificial Sequence
  Synthetic Polypeptide
PFPQPEQPI

SEQ ID NO: 987 - protein, 9 aa, Artificial Sequence
  Synthetic Polypeptide
PQPEQPIPV

SEQ ID NO: 988 - protein, 9 aa, Artificial Sequence
  Synthetic Polypeptide
EQPIPVQPE

SEQ ID NO: 989 - protein, 9 aa, Artificial Sequence
  Synthetic Polypeptide
PFPQPEQPT

SEQ ID NO: 990 - protein, 9 aa, Artificial Sequence
  Synthetic Polypeptide
PQPEQPTPI

SEQ ID NO: 991 - protein, 9 aa, Artificial Sequence
  Synthetic Polypeptide
EQPTPIQPE

SEQ ID NO: 992 - protein, 9 aa, Artificial Sequence
  Synthetic Polypeptide
PQPEQPFPL

SEQ ID NO: 993 - protein, 9 aa, Artificial Sequence
  Synthetic Polypeptide
EQPFPLQPE

SEQ ID NO: 994 - protein, 9 aa, Artificial Sequence
  Synthetic Polypeptide
EGSFQPSQE

SEQ ID NO: 995 - protein, 9 aa, Artificial Sequence
  Synthetic Polypeptide
QGYYPTSPQ

SEQ ID NO: 996 - protein, 9 aa, Artificial Sequence
  Synthetic Polypeptide
EQPEQPFPE

SEQ ID NO: 997 - protein, 9 aa, Artificial Sequence
  Synthetic Polypeptide
PFSEQEQPV

SEQ ID NO: 998 - protein, 9 aa, Artificial Sequence
  Synthetic Polypeptide
PYPQPELPY

SEQ ID NO: 999 - protein, 9 aa, Artificial Sequence
  Synthetic Polypeptide
EQPFPEQPI

SEQ ID NO: 1000 - protein, 9 aa, Artificial Sequence
  Synthetic Polypeptide
PFPEQPIPE

SEQ ID NO: 1001 - protein, 9 aa, Artificial Sequence
  Synthetic Polypeptide
PYPEQPQPF

SEQ ID NO: 1002 - protein, 9 aa, Artificial Sequence
  Synthetic Polypeptide
PQPYPEQPQ

SEQ ID NO: 1003 - protein, 12 aa, Artificial Sequence
  Synthetic Polypeptide
PFPQPELPYPQP

SEQ ID NO: 1004 - protein, 12 aa, Artificial Sequence
  Synthetic Polypeptide
PFPQPEQPFPWQ

SEQ ID NO: 1005 - protein, 12 aa, Artificial Sequence
  Synthetic Polypeptide
EQPIPEQPQPYP

SEQ ID NO: 1006 - protein, 12 aa, Artificial Sequence
  Synthetic Polypeptide
PFPQPEQPIPVQ

SEQ ID NO: 1007 - protein, 12 aa, Artificial Sequence
  Synthetic Polypeptide
PEQPIPVQPEQS

SEQ ID NO: 1008 - protein, 12 aa, Artificial Sequence
  Synthetic Polypeptide
PFPQPEQPTPIQ

SEQ ID NO: 1009 - protein, 12 aa, Artificial Sequence
  Synthetic Polypeptide
PEQPTPIQPEQP

SEQ ID NO: 1010 - protein, 12 aa, Artificial Sequence
  Synthetic Polypeptide
PFPQPEQPFPLQ

SEQ ID NO: 1011 - protein, 12 aa, Artificial Sequence
  Synthetic Polypeptide
PEQPFPLQPEQP

SEQ ID NO: 1012 - protein, 12 aa, Artificial Sequence
  Synthetic Polypeptide
GEGSFQPSQENP

SEQ ID NO: 1013 - protein, 12 aa, Artificial Sequence
  Synthetic Polypeptide
QQGYYPTSPQQS

SEQ ID NO: 1014 - protein, 12 aa, Artificial Sequence
  Synthetic Polypeptide
PEQPEQPFPEQP

SEQ ID NO: 1015 - protein, 12 aa, Artificial Sequence
  Synthetic Polypeptide
PPFSEQEQPVLP

SEQ ID NO: 1016 - protein, 12 aa, Artificial Sequence
  Synthetic Polypeptide
PYPQPELPYPQP

SEQ ID NO: 1017 - protein, 12 aa, Artificial Sequence
  Synthetic Polypeptide
EQPFPEQPIPEQ

SEQ ID NO: 1018 - protein, 12 aa, Artificial Sequence
  Synthetic Polypeptide
PQPYPEQPQPFP

SEQ ID NO: 1019 - protein, 16 aa, Artificial Sequence
  Synthetic Polypeptide
XLQPFPQPELPYPQPQ

SEQ ID NO: 1020 - protein, 15 aa, Artificial Sequence
  Synthetic Polypeptide
XQPFPQPEQPFPWQP

SEQ ID NO: 1021 - protein, 16 aa, Artificial Sequence
  Synthetic Polypeptide
XPEQPIPEQPQPYPQQ

SEQ ID NO: 1022 - protein, 16 aa, Artificial Sequence
  Synthetic Polypeptide
XLQPFPQPELPYPQPQ

SEQ ID NO: 1023 - protein, 15 aa, Artificial Sequence
  Synthetic Polypeptide
XQPFPQPEQPFPWQP

SEQ ID NO: 1024 - protein, 16 aa, Artificial Sequence
  Synthetic Polypeptide
XPEQPIPEQPQPYPQQ

SEQ ID NO: 1025 - protein, 18 aa, Artificial Sequence
  Synthetic Polypeptide
XQPFPQPEQPIPVQPEQS

SEQ ID NO: 1026 - protein, 18 aa, Artificial Sequence
  Synthetic Polypeptide
XQPFPQPEQPTPIQPEQP

SEQ ID NO: 1027 - protein, 18 aa, Artificial Sequence
  Synthetic Polypeptide
XQPFPQPEQPFPLQPEQP

SEQ ID NO: 1028 - protein, 14 aa, Artificial Sequence
  Synthetic Polypeptide
XQPFPQPEQPFSQQ

SEQ ID NO: 1029 - protein, 15 aa, Artificial Sequence
  Synthetic Polypeptide
XPQPYPEQPQPFPQQ

SEQ ID NO: 1030 - protein, 16 aa, Artificial Sequence
  Synthetic Polypeptide
XQPFPEQPEQIIPQQP

SEQ ID NO: 1031 - protein, 15 aa, Artificial Sequence
  Synthetic Polypeptide
XSGEGSFQPSQENPQ

SEQ ID NO: 1032 - protein, 15 aa, Artificial Sequence
  Synthetic Polypeptide
XGQQGYYPTSPQQSG

SEQ ID NO: 1033 - protein, 15 aa, Artificial Sequence
  Synthetic Polypeptide
XPEQPEQPFPEQPQQ

SEQ ID NO: 1034 - protein, 15 aa, Artificial Sequence
  Synthetic Polypeptide
XQPPFSEQEQPVLPQ

SEQ ID NO: 1035 - protein, 15 aa, Artificial Sequence
  Synthetic Polypeptide
XQPFPQPEQPFPWQP

SEQ ID NO: 1036 - protein, 18 aa, Artificial Sequence
  Synthetic Polypeptide
XQPFPQPEQPIPVQPEQS

SEQ ID NO: 1037 - protein, 18 aa, Artificial Sequence
  Synthetic Polypeptide
XQPFPQPEQPTPIQPEQP

SEQ ID NO: 1038 - protein, 18 aa, Artificial Sequence
  Synthetic Polypeptide
XQPFPQPEQPFPLQPEQP

SEQ ID NO: 1039 - protein, 14 aa, Artificial Sequence
  Synthetic Polypeptide
XQPFPQPEQPFSQQ

SEQ ID NO: 1040 - protein, 15 aa, Artificial Sequence
  Synthetic Polypeptide
XPQPYPEQPQPFPQQ

SEQ ID NO: 1041 - protein, 16 aa, Artificial Sequence
  Synthetic Polypeptide
XQPFPEQPEQIIPQQP

SEQ ID NO: 1042 - protein, 15 aa, Artificial Sequence
  Synthetic Polypeptide
XSGEGSFQPSQENPQ

SEQ ID NO: 1043 - protein, 15 aa, Artificial Sequence
  Synthetic Polypeptide
XGQQGYYPTSPQQSG

SEQ ID NO: 1044 - protein, 15 aa, Artificial Sequence
  Synthetic Polypeptide
XPEQPEQPFPEQPQQ

SEQ ID NO: 1045 - protein, 15 aa, Artificial Sequence
  Synthetic Polypeptide
XQPPFSEQEQPVLPQ

SEQ ID NO: 1046 - protein, 19 aa, Artificial Sequence
  Synthetic Polypeptide
XPEQPFPEQPIPEQPQPYP

SEQ ID NO: 1047 - protein, 15 aa, Artificial Sequence
  Synthetic Polypeptide
XQPYPQPELPYPQPQ

SEQ ID NO: 1048 - protein, 15 aa, Artificial Sequence
  Synthetic Polypeptide
XQPFPQPELPYPYPQ

SEQ ID NO: 1049 - protein, 16 aa, Artificial Sequence
  Synthetic Polypeptide
XLQPFPQPELPYPQPQ

SEQ ID NO: 1050 - protein, 15 aa, Artificial Sequence
  Synthetic Polypeptide
XQPFPQPEQPFPWQP

SEQ ID NO: 1051 - protein, 16 aa, Artificial Sequence
  Synthetic Polypeptide
XPEQPIPEQPQPYPQQ

SEQ ID NO: 1052 - protein, 18 aa, Artificial Sequence
  Synthetic Polypeptide
XQPFPQPEQPIPVQPEQS

SEQ ID NO: 1053 - protein, 18 aa, Artificial Sequence
  Synthetic Polypeptide
XQPFPQPEQPTPIQPEQP

SEQ ID NO: 1054 - protein, 18 aa, Artificial Sequence
  Synthetic Polypeptide
XQPFPQPEQPFPLQPEQP

SEQ ID NO: 1055 - protein, 14 aa, Artificial Sequence
  Synthetic Polypeptide
XQPFPQPEQPFSQQ

SEQ ID NO: 1056 - protein, 15 aa, Artificial Sequence
  Synthetic Polypeptide
XPQPYPEQPQPFPQQ

SEQ ID NO: 1057 - protein, 15 aa, Artificial Sequence
  Synthetic Polypeptide
XSGEGSFQPSQENPQ

SEQ ID NO: 1058 - protein, 15 aa, Artificial Sequence
  Synthetic Polypeptide
XGQQGYYPTSPQQSG

SEQ ID NO: 1059 - protein, 15 aa, Artificial Sequence
  Synthetic Polypeptide
XPEQPEQPFPEQPQQ

SEQ ID NO: 1060 - protein, 16 aa, Artificial Sequence
  Synthetic Polypeptide
XQPFPEQPEQIIPQQP

SEQ ID NO: 1061 - protein, 15 aa, Artificial Sequence
  Synthetic Polypeptide
XQPPFSEQEQPVLPQ

SEQ ID NO: 1062 - protein, 19 aa, Artificial Sequence
  Synthetic Polypeptide
XPEQPFPEQPIPEQPQPYP

SEQ ID NO: 1063 - protein, 15 aa, Artificial Sequence
  Synthetic Polypeptide
XQPYPQPELPYPQPQ

SEQ ID NO: 1064 - protein, 15 aa, Artificial Sequence
  Synthetic Polypeptide
XQPFPQPELPYPYPQ

SEQ ID NO: 1065 - protein, 16 aa, Artificial Sequence
  Synthetic Polypeptide
XPQEQPFPEQPIPEQP

SEQ ID NO: 1066 - protein, 16 aa, Artificial Sequence
  Synthetic Polypeptide
XQPQPYPEQPQPFPQQ

SEQ ID NO: 1067 - protein, 9 aa, Artificial Sequence
  Synthetic Polypeptide
PFPQPELPY

SEQ ID NO: 1068 - protein, 9 aa, Artificial Sequence
  Synthetic Polypeptide
PQPELPYPQ

SEQ ID NO: 1069 - protein, 9 aa, Artificial Sequence
  Synthetic Polypeptide
PFPQPEQPF

SEQ ID NO: 1070 - protein, 9 aa, Artificial Sequence
  Synthetic Polypeptide
PQPEQPFPW

SEQ ID NO: 1071 - protein, 9 aa, Artificial Sequence
  Synthetic Polypeptide
EQPIPEQPQ

SEQ ID NO: 1072 - protein, 9 aa, Artificial Sequence
  Synthetic Polypeptide
PIPEQPQPY

SEQ ID NO: 1073 - protein, 9 aa, Artificial Sequence
  Synthetic Polypeptide
PFPQPEQPI

SEQ ID NO: 1074 - protein, 9 aa, Artificial Sequence
  Synthetic Polypeptide
PQPEQPIPV

SEQ ID NO: 1075 - protein, 9 aa, Artificial Sequence
  Synthetic Polypeptide
EQPIPVQPE

SEQ ID NO: 1076 - protein, 9 aa, Artificial Sequence
  Synthetic Polypeptide
PFPQPEQPT

SEQ ID NO: 1077 - protein, 9 aa, Artificial Sequence
  Synthetic Polypeptide
PQPEQPTPI

SEQ ID NO: 1078 - protein, 9 aa, Artificial Sequence
  Synthetic Polypeptide
EQPTPIQPE

SEQ ID NO: 1079 - protein, 9 aa, Artificial Sequence
  Synthetic Polypeptide
PFPQPEQPF

SEQ ID NO: 1080 - protein, 9 aa, Artificial Sequence
  Synthetic Polypeptide
PQPEQPFPL

SEQ ID NO: 1081 - protein, 9 aa, Artificial Sequence
  Synthetic Polypeptide
EQPFPLQPE

SEQ ID NO: 1082 - protein, 9 aa, Artificial Sequence
  Synthetic Polypeptide
PFPQPEQPF

SEQ ID NO: 1083 - protein, 9 aa, Artificial Sequence
  Synthetic Polypeptide
PQPEQPFSQ

SEQ ID NO: 1084 - protein, 9 aa, Artificial Sequence
  Synthetic Polypeptide
PYPEQPQPF

SEQ ID NO: 1085 - protein, 9 aa, Artificial Sequence
  Synthetic Polypeptide
EGSFQPSQE

SEQ ID NO: 1086 - protein, 9 aa, Artificial Sequence
  Synthetic Polypeptide
QGYYPTSPQ

SEQ ID NO: 1087 - protein, 9 aa, Artificial Sequence
  Synthetic Polypeptide
EQPEQPFPE

SEQ ID NO: 1088 - protein, 9 aa, Artificial Sequence
  Synthetic Polypeptide
EQPFPEQPQ

SEQ ID NO: 1089 - protein, 9 aa, Artificial Sequence
  Synthetic Polypeptide
PFPEQPEQI

SEQ ID NO: 1090 - protein, 9 aa, Artificial Sequence
  Synthetic Polypeptide
PFSEQEQPV

SEQ ID NO: 1091 - protein, 9 aa, Artificial Sequence
  Synthetic Polypeptide
EQPFPEQPI

SEQ ID NO: 1092 - protein, 9 aa, Artificial Sequence
  Synthetic Polypeptide
PFPEQPIPE

SEQ ID NO: 1093 - protein, 9 aa, Artificial Sequence
  Synthetic Polypeptide
EQPIPEQPQ

SEQ ID NO: 1094 - protein, 9 aa, Artificial Sequence
  Synthetic Polypeptide
PIPEQPQPY

SEQ ID NO: 1095 - protein, 9 aa, Artificial Sequence
  Synthetic Polypeptide
PYPQPELPY

SEQ ID NO: 1096 - protein, 9 aa, Artificial Sequence
  Synthetic Polypeptide
PQPELPYPQ

SEQ ID NO: 1097 - protein, 9 aa, Artificial Sequence
  Synthetic Polypeptide
PFPQPELPY

SEQ ID NO: 1098 - protein, 9 aa, Artificial Sequence
  Synthetic Polypeptide
PQPELPYPY

SEQ ID NO: 1099 - protein, 9 aa, Artificial Sequence
  Synthetic Polypeptide
EQPFPEQPI

SEQ ID NO: 1100 - protein, 9 aa, Artificial Sequence
  Synthetic Polypeptide
PFPEQPIPE

SEQ ID NO: 1101 - protein, 9 aa, Artificial Sequence
  Synthetic Polypeptide
PQPYPEQPQ

SEQ ID NO: 1102 - protein, 9 aa, Artificial Sequence
  Synthetic Polypeptide
PYPEQPQPF

SEQ ID NO: 1103 - protein, 16 aa, Artificial Sequence
  Synthetic Polypeptide
XLQPFPQPELPYPQPQ

SEQ ID NO: 1104 - protein, 15 aa, Artificial Sequence
  Synthetic Polypeptide
XQPFPQPEQPFPWQP

SEQ ID NO: 1105 - protein, 16 aa, Artificial Sequence
  Synthetic Polypeptide
XPEQPIPEQPQPYPQQ

SEQ ID NO: 1106 - protein, 13 aa, Artificial Sequence
  Synthetic Polypeptide
XPFPQPELPYPQP

SEQ ID NO: 1107 - protein, 13 aa, Artificial Sequence
  Synthetic Polypeptide
XPFPQPEQPFPWQ

SEQ ID NO: 1108 - protein, 13 aa, Artificial Sequence
  Synthetic Polypeptide
XEQPIPEQPQPYP

SEQ ID NO: 1109 - protein, 13 aa, Artificial Sequence
  Synthetic Polypeptide
XPFPQPEQPIPVQ

SEQ ID NO: 1110 - protein, 13 aa, Artificial Sequence
  Synthetic Polypeptide
XPEQPIPVQPEQS

SEQ ID NO: 1111 - protein, 13 aa, Artificial Sequence
  Synthetic Polypeptide
XPFPQPEQPTPIQ

SEQ ID NO: 1112 - protein, 13 aa, Artificial Sequence
  Synthetic Polypeptide
XPEQPTPIQPEQP

SEQ ID NO: 1113 - protein, 13 aa, Artificial Sequence
  Synthetic Polypeptide
XPFPQPEQPFPLQ

SEQ ID NO: 1114 - protein, 13 aa, Artificial Sequence
  Synthetic Polypeptide
XPEQPFPLQPEQP

SEQ ID NO: 1115 - protein, 13 aa, Artificial Sequence
  Synthetic Polypeptide
XGEGSFQPSQENP

SEQ ID NO: 1116 - protein, 13 aa, Artificial Sequence
  Synthetic Polypeptide
XQQGYYPTSPQQS

SEQ ID NO: 1117 - protein, 13 aa, Artificial Sequence
  Synthetic Polypeptide
XPEQPEQPFPEQP

SEQ ID NO: 1118 - protein, 13 aa, Artificial Sequence
  Synthetic Polypeptide
XPPFSEQEQPVLP

SEQ ID NO: 1119 - protein, 13 aa, Artificial Sequence
  Synthetic Polypeptide
XPYPQPELPYPQP

SEQ ID NO: 1120 - protein, 13 aa, Artificial Sequence
  Synthetic Polypeptide
XEQPFPEQPIPEQ

SEQ ID NO: 1121 - protein, 13 aa, Artificial Sequence
  Synthetic Polypeptide
XPQPYPEQPQPFP

SEQ ID NO: 1122 - protein, 9 aa, Artificial Sequence
  Synthetic Polypeptide
PFPQPELPY

SEQ ID NO: 1123 - protein, 9 aa, Artificial Sequence
  Synthetic Polypeptide
PQPELPYPQ

SEQ ID NO: 1124 - protein, 9 aa, Artificial Sequence
  Synthetic Polypeptide
PFPQPEQPF

SEQ ID NO: 1125 - protein, 9 aa, Artificial Sequence
  Synthetic Polypeptide
PQPEQPFPW

SEQ ID NO: 1126 - protein, 9 aa, Artificial Sequence
  Synthetic Polypeptide
EQPIPEQPQ

SEQ ID NO: 1127 - protein, 9 aa, Artificial Sequence
  Synthetic Polypeptide
PIPEQPQPY

SEQ ID NO: 1128 - protein, 9 aa, Artificial Sequence
  Synthetic Polypeptide
PFPQPEQPI

SEQ ID NO: 1129 - protein, 9 aa, Artificial Sequence
  Synthetic Polypeptide
PQPEQPIPV

SEQ ID NO: 1130 - protein, 9 aa, Artificial Sequence
  Synthetic Polypeptide
EQPIPVQPE

SEQ ID NO: 1131 - protein, 9 aa, Artificial Sequence
  Synthetic Polypeptide
PFPQPEQPT

SEQ ID NO: 1132 - protein, 9 aa, Artificial Sequence
  Synthetic Polypeptide
PQPEQPTPI

SEQ ID NO: 1133 - protein, 9 aa, Artificial Sequence
  Synthetic Polypeptide
EQPTPIQPE

SEQ ID NO: 1134 - protein, 9 aa, Artificial Sequence
  Synthetic Polypeptide
PQPEQPFPL

SEQ ID NO: 1135 - protein, 9 aa, Artificial Sequence
  Synthetic Polypeptide
EQPFPLQPE

SEQ ID NO: 1136 - protein, 9 aa, Artificial Sequence
  Synthetic Polypeptide
EGSFQPSQE

SEQ ID NO: 1137 - protein, 9 aa, Artificial Sequence
  Synthetic Polypeptide
QGYYPTSPQ

SEQ ID NO: 1138 - protein, 9 aa, Artificial Sequence
  Synthetic Polypeptide
EQPEQPFPE

SEQ ID NO: 1139 - protein, 9 aa, Artificial Sequence
  Synthetic Polypeptide
PFSEQEQPV

SEQ ID NO: 1140 - protein, 9 aa, Artificial Sequence
  Synthetic Polypeptide
PYPQPELPY

SEQ ID NO: 1141 - protein, 9 aa, Artificial Sequence
  Synthetic Polypeptide
PQPELPYPQ

SEQ ID NO: 1142 - protein, 9 aa, Artificial Sequence
  Synthetic Polypeptide
EQPFPEQPI

SEQ ID NO: 1143 - protein, 9 aa, Artificial Sequence
  Synthetic Polypeptide
PFPEQPIPE

SEQ ID NO: 1144 - protein, 9 aa, Artificial Sequence
  Synthetic Polypeptide
PYPEQPQPF

SEQ ID NO: 1145 - protein, 9 aa, Artificial Sequence
  Synthetic Polypeptide
PQPYPEQPQ

(19) United States
(12) Patent Application Publication
(10) Pub. No.: US 2017/0097346 A1
(43) Pub. Date: Apr. 6, 2017

(54) USE OF INTERLEUKIN-2 FOR DIAGNOSIS OF CELIAC DISEASE

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 15/306,219

(22) PCT Filed: Apr. 24, 2015
(86) PCT No.: PCT/US2015/027477

§ 371 Date: Oct. 24, 2016

Related U.S. Application Data
(60) Provisional application No. 62/043,395, filed on Aug. 28, 2014.
(60) Provisional application No. 62/014,666, filed on Jun. 19, 2014.
(60) Provisional application No. 62/014,676, filed on Jun. 19, 2014.
(60) Provisional application No. 62/014,681, filed on Jun. 19, 2014.
(60) Provisional application No. 62/014,373, filed on Jun. 19, 2014.
(60) Provisional application No. 62/014,401, filed on Jun. 19, 2014.
(60) Provisional application No. 62/011,794, filed on Jun. 13, 2014.
(60) Provisional application No. 62/011,493, filed on Jun. 12, 2014.
(60) Provisional application No. 62/011,540, filed on Jun. 12, 2014.
(60) Provisional application No. 62/011,508, filed on Jun. 12, 2014.
(60) Provisional application No. 62/011,561, filed on Jun. 12, 2014.
(60) Provisional application No. 62/011,566, filed on Jun. 12, 2014.
(60) Provisional application No. 62/009,090, filed on Jun. 6, 2014.
(60) Provisional application No. 62/009,146, filed on Jun. 6, 2014.
(60) Provisional application No. 61/984,043, filed on Apr. 25, 2014.
(60) Provisional application No. 61/983,989, filed on Apr. 24, 2014.
(60) Provisional application No. 61/983,993, filed on Apr. 24, 2014.
(60) Provisional application No. 61/983,981, filed on Apr. 24, 2014.
(60) Provisional application No. 61/984,028, filed on Apr. 24, 2014.

(51) Int. Cl. G01N 33/564; G01N 33/68

(52) U.S. Cl. CPC G01N 33/564; G01N 33/6869; G01N 2333/55; G01N 2800/50; G01N 2800/06; G01N 2333/415

(57) ABSTRACT

Provided herein are methods of identifying a subject having or at risk for having Celiac disease by determining a level of IL-2 in a sample from a subject.

### RELATED APPLICATIONS

This application claims the benefit under 35 U.S.C. §119(e) of U.S. provisional application No. 61/983,981, filed Apr. 24, 2014, U.S. provisional application No. 62/011,561, filed Jun. 12, 2014, U.S. provisional application No. 62/014,676, filed Jun. 19, 2014, U.S. provisional application No. 62/057,152, filed Sep. 29, 2014, U.S. provisional application No. 62/115,925, filed Feb. 13, 2015, U.S. provisional application No. 61/984,028, filed Apr. 24, 2014, U.S. provisional application No. 61/984,043, filed Apr. 25, 2014, U.S. provisional application No. 62/011,566, filed Jun. 12, 2014, U.S. provisional application No. 62/014,681, filed Jun. 19, 2014, U.S. provisional application No. 62/057,163, filed Sep. 29, 2014, U.S. provisional application No. 62/115,897, filed Feb. 13, 2015, U.S. provisional application No. 61/983,989, filed Apr. 24, 2014, U.S. provisional application No. 62/014,666, filed Jun. 19, 2014, U.S. provisional application No. 62/009,146, filed Jun. 6, 2014, U.S. provisional application No. 62/043,386, filed Aug. 28, 2014, U.S. provisional application No. 62/115,963, filed Feb. 13, 2015, U.S. provisional application No. 61/983,993, filed Apr. 24, 2014, U.S. provisional application No. 62/011,508, filed Jun. 12, 2014, U.S. provisional application No. 62/116,052, filed Feb. 13, 2015, U.S. provisional application No. 62/043,395, filed Aug. 28, 2014, U.S. provisional application No. 62/082,832, filed Nov. 21, 2014, U.S. provisional application No. 62/009,090, filed Jun. 6, 2014, U.S. provisional application No. 62/014,373, filed Jun. 19, 2014, U.S. provisional application No. 62/043,390, filed Aug. 28, 2014, U.S. provisional application No. 62/116,002, filed Feb. 13, 2015, U.S. provisional application No. 62/011,493, filed Jun. 12, 2014, U.S. provisional application No. 62/011,794, filed Jun. 13, 2014, U.S. provisional application No. 62/014,401, filed Jun. 19, 2014, U.S. provisional application No. 62/116,027, filed Feb. 13, 2015, and U.S. provisional application No. 62/011,540, filed Jun. 12, 2014, the contents of each of which are incorporated by reference herein in their entirety.

### BACKGROUND

Celiac disease is an autoimmune disorder of the small intestine that occurs in people of all ages. Celiac disease causes damage to the villi of the small intestine due to an inappropriate immune response to gluten peptides, leading to malabsorption and an increased risk of intestinal cancer. Identifying subjects with Celiac disease is important for ensuring that such Celiac disease subjects receive proper treatment.

### SUMMARY

Aspects of the disclosure relate to methods of identifying (e.g., diagnosing) a subject as having or at risk of having Celiac disease by determining a level of Interleukin-2 (IL-2) in a sample from the subject.

In some aspects, the disclosure relates to a method of identifying a subject having or at risk for having celiac disease, the method comprising (a) determining a level of IL-2 in a sample comprising a T cell from the subject, which sample has been contacted with a composition comprising at least one gluten peptide; and (b) assessing whether or not the subject has or is at risk of having Celiac disease.

In some embodiments, the determining step comprises (i) contacting the sample comprising the T cell with the composition comprising at least one gluten peptide; and (ii) measuring the level of IL-2 in the sample comprising the T cell after the contacting. In some embodiments, measuring the level of IL-2 comprises an enzyme-linked immunosorbent assay (ELISA) or a multiplex bead-based immunoassay.

In some embodiments, the method further comprises: (c) comparing the level of IL-2 in the sample with a control level of IL-2. In some embodiments, the assessing comprises (i) identifying the subject as having or at risk of having Celiac disease if the IL-2 level is elevated compared to a control level of IL-2; or (ii) not having or not at risk of having Celiac disease if the IL-2 level is reduced compared to the control level of IL-2 or the same as the control level of IL-2.

In some embodiments, the control level of IL-2 is a pre-determined threshold. In some embodiments, the control level of IL-2 is 3 pg/mL. In some embodiments, the control level of IL-2 is the level of IL-2 in another sample comprising a T cell obtained from the subject that is not contacted with the composition comprising at least one gluten peptide. In some embodiments, the sample comprising the T cell is a sample that comprises whole blood or peripheral blood mononuclear cells.

In some embodiments, the composition comprises at least one (e.g., one, two, three, four, five, six, seven, eight, nine, ten, eleven, twelve, thirteen, fourteen, fifteen, or sixteen) peptide(s), the at least one (e.g., one, two, three, four, five, six, seven, eight, nine, ten, eleven, twelve, thirteen, fourteen, fifteen, or sixteen) peptide(s) comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23). In some embodiments, the composition comprises at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 15), QGYYPTSPQ (SEQ ID NO: 16), EQPEQPFPE (SEQ ID NO: 17), PFSEQEQPV (SEQ ID NO: 18), PYPQPELPY (SEQ ID NO: 19), EQPFPEQPI (SEQ ID NO: 20), PFPEQPIPE (SEQ ID NO: 21), PYPEQPQPF (SEQ ID NO: 22), and PQPYPEQPQ (SEQ ID NO: 23).

In some embodiments, the composition comprises (or consists of) at least one (or consists of) of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 15);

(k) a eleventh peptide comprising the amino acid sequence QGYYPTSPQ (SEQ ID NO: 16);

(l) a twelfth peptide comprising the amino acid sequence EQPEQPFPE (SEQ ID NO: 17);

(m) a thirteenth peptide comprising the amino acid sequence PFSEQEQPV (SEQ ID NO: 18);

(n) a fourteenth peptide comprising the amino acid sequence PYPQPELPY (SEQ ID NO: 19);

(o) a fifteenth peptide comprising the amino acid sequence EQPFPEQPI (SEQ ID NO: 20) and the amino acid sequence PFPEQPIPE (SEQ ID NO: 21); and

(p) a sixteenth peptide comprising the amino acid sequence PYPEQPQPF (SEQ ID NO: 22) and the amino acid sequence PQPYPEQPQ (SEQ ID NO: 23).

In some embodiments:

(a) the first peptide comprises the amino acid sequence PFPQPELPYPQP (SEQ ID NO: 24);

(b) the second peptide comprises the amino acid sequence PFPQPEQPFPWQ (SEQ ID NO: 25);

(c) the third peptide comprises the amino acid sequence EQPIPEQPQPYP (SEQ ID NO: 26);

(d) the fourth peptide comprises the amino acid sequence PFPQPEQPIPVQ (SEQ ID NO: 27);

(e) the fifth peptide comprises the amino acid sequence PEQPIPVQPEQS (SEQ ID NO: 28);

(f) the sixth peptide comprises the amino acid sequence PFPQPEQPTPIQ (SEQ ID NO: 29);

(g) the seventh peptide comprises the amino acid sequence PEQPTPIQPEQP (SEQ ID NO: 30);

(h) the eighth peptide comprises the amino acid sequence PFPQPEQPFPLQ (SEQ ID NO: 31);

(i) the ninth peptide comprises the amino acid sequence PEQPFPLQPEQP (SEQ ID NO: 32);

(j) the tenth peptide comprises the amino acid sequence GEGSFQPSQENP (SEQ ID NO: 33);

(k) the eleventh peptide comprises the amino acid sequence QQGYYPTSPQQS (SEQ ID NO: 34);

(l) the twelfth peptide comprises the amino acid sequence PEQPEQPFPEQP (SEQ ID NO: 35);

(m) the thirteenth peptide comprises the amino acid sequence PPFSEQEQPVLP (SEQ ID NO: 36);

(n) the fourteenth peptide comprises the amino acid sequence PYPQPELPYPQP (SEQ ID NO: 37);

(o) the fifteenth peptide comprises the amino acid sequence EQPFPEQPIPEQ (SEQ ID NO: 38); and

(p) the sixteenth peptide comprises the amino acid sequence PQPYPEQPQPFP (SEQ ID NO: 39).

In some embodiments, the composition comprises (or consists of) at least four (e.g., four, five, six, seven, eight, nine, ten, eleven, twelve, thirteen, fourteen, fifteen or sixteen) of the peptides. In some embodiments, the composition comprises (or consists of) the peptides in (a)-(p).

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 12 to 30 amino acids in length. In some embodiments, each of the peptides is 13 amino acids in length.

In some embodiments of any one of the compositions provided, each of the peptides are present in an amount of 2.5 ug/mL in the composition. In some embodiments of any one of the compositions provided, each of the peptides are present in an amount of 5 ug/mL in the composition. In some embodiments of any one of the compositions provided, each of the peptides are present in an amount of 10 ug/mL in the composition. In some embodiments of any one of the compositions provided, each of the peptides are present in an amount of 20 ug/mL in the composition. In some embodiments of any one of the compositions provided, each of the peptides are present in an amount of 25 ug/mL in the composition. In some embodiments of any one of the compositions provided, each of the peptides are present in an amount of 50 ug/mL in the composition. In some embodiments of any one of the compositions provided, each of the peptides are present in an amount of 5 uM (micromolar) in the composition. In some embodiments of any one of the compositions provided, each of the peptides are present in an amount of 10 uM in the composition. In some embodiments of any one of the compositions provided, each of the peptides are present in an amount of 25 uM in the composition. In some embodiments of any one of the compositions provided, each of the peptides are present in an amount of 50 uM in the composition.

In some embodiments, the composition comprises at least one (e.g., one, two, three, four, five, six, seven, eight, nine, ten, eleven, twelve, thirteen, fourteen, fifteen, or sixteen) peptide(s), the at least one (e.g., one, two, three, four, five, six, seven, eight, nine, ten, eleven, twelve, thirteen, fourteen, fifteen, or sixteen) peptide(s) comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 43), EQPFPEQPI (SEQ ID NO: 20), PFPEQPIPE (SEQ ID NO: 21), PYPQPELPY (SEQ ID NO: 19), PQPELPYPY (SEQ ID NO: 44), and PQPYPEQPQ (SEQ ID NO: 23). In some embodiments, the composition comprises at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 22), EGSFQPSQE (SEQ ID NO: 15), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 43), EQPFPEQPI (SEQ ID NO: 20), PFPEQPIPE (SEQ ID NO: 21), PYPQPELPY (SEQ ID NO: 19), PQPELPYPY (SEQ ID NO: 44), and PQPYPEQPQ (SEQ ID NO: 23). In some embodiments, the compositions comprises at least one (e.g., one, two, three, four, five, six, seven, eight, nine, ten, eleven, twelve, thirteen, fourteen, fifteen, or sixteen) peptide(s) comprising the amino acid sequences PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6) and at least one (e.g., one, two, three, four, five, six, seven, eight, nine, ten, eleven, twelve, thirteen, fourteen, fifteen, sixteen, seventeen, eighteen, nineteen, twenty, twenty-one or more) further amino acid sequence(s) selected from PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 45), PYPEQPQPF (SEQ ID NO: 46), EGSFQPSQE (SEQ ID NO: 47), QGYYPTSPQ (SEQ ID NO: 48), EQPEQPFPE (SEQ ID NO: 49), EQPFPEQPQ (SEQ ID NO: 50), PFPEQPEQI (SEQ ID NO: 51), PFSEQEQPV (SEQ ID NO: 52), EQPFPEQPI (SEQ ID NO: 53, PFPEQPIPE (SEQ ID NO: 54), PYPQPELPY (SEQ ID NO: 55), PQPELPYPY (SEQ ID NO: 56), and PQPYPEQPQ (SEQ ID NO: 57). In some embodiments, the composition comprises at least one peptide comprising the amino acid sequences EQPFPEQPI (SEQ ID NO: 58), PFPEQPIPE (SEQ ID NO: 59), EQPIPEQPQ (SEQ ID NO: 60), and PIPEQPQPY (SEQ ID NO: 61) (e.g., the composition comprises at least one peptide comprising the amino acid sequence PEQPFPEQPIPEQPQPYP (SEQ ID NO: 62)).

In some embodiments, the composition comprises (or consists of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 63) and the amino acid sequence PQPEQPFSQ (SEQ ID NO: 64);

(h) an eighth peptide comprising the amino acid sequence PYPEQPQPF (SEQ ID NO: 65);

(i) a ninth peptide comprising the amino acid sequence PFPEQPEQI (SEQ ID NO: 66);

(j) a tenth peptide comprising the amino acid sequence EGSFQPSQE (SEQ ID NO: 67);

(k) an eleventh peptide comprising the amino acid sequence QGYYPTSPQ (SEQ ID NO: 68);

(l) a twelfth peptide comprising the amino acid sequence EQPEQPFPE (SEQ ID NO: 69) and the amino acid sequence EQPFPEQPQ (SEQ ID NO: 70);

(m) a thirteenth peptide comprising the amino acid sequence PFSEQEQPV (SEQ ID NO: 71);

(n) a fourteenth peptide comprising the amino acid sequence EQPFPEQPI (SEQ ID NO: 72), the amino acid sequence PFPEQPIPE (SEQ ID NO: 73), the amino acid sequence EQPIPEQPQ (SEQ ID NO: 74), and the amino acid sequence PIPEQPQPY (SEQ ID NO: 75);

(o) a fifteenth peptide comprising the amino acid sequence PQPELPYPQ (SEQ ID NO: 76) and the amino acid sequence PYPQPELPY (SEQ ID NO: 77);

(p) a sixteenth peptide comprising the amino acid sequence PFPQPELPY (SEQ ID NO: 78) and the amino acid sequence PQPELPYPY (SEQ ID NO: 79);

(q) a seventeenth peptide comprising the amino acid sequence EQPFPEQPI (SEQ ID NO: 80) and the amino acid sequence PFPEQPIPE (SEQ ID NO: 81); and

(r) an eighteenth peptide comprising the amino acid sequence PQPYPEQPQ (SEQ ID NO: 82) and the amino acid sequence PYPEQPQPF (SEQ ID NO: 83).

In some embodiments,

(a) the first peptide comprises the amino acid sequence LQPFPQPELPYPQPQ (SEQ ID NO: 84);

(b) the second peptide comprises the amino acid sequence QPFPQPEQPFPWQP (SEQ ID NO: 85);

(c) the third peptide comprises the amino acid sequence PEQPIPEQPQPYPQQ (SEQ ID NO: 86);

(d) the fourth peptide comprises the amino acid sequence QPFPQPEQPIPVQPEQS (SEQ ID NO: 87);

(e) the fifth peptide comprises the amino acid sequence QPFPQPEQPTPIQPEQP (SEQ ID NO: 88);

(f) the sixth peptide comprises the amino acid sequence QPFPQPEQPFPLQPEQP (SEQ ID NO: 89);

(g) the seventh peptide comprises the amino acid sequence QPFPQPEQPFSQQ (SEQ ID NO: 90);

(h) the eighth peptide comprises the amino acid sequence PQPYPEQPQPFPQQ (SEQ ID NO: 91);

(i) the ninth peptide comprises the amino acid sequence QPFPEQPEQIIPQQP (SEQ ID NO: 92);

(j) the tenth peptide comprises the amino acid sequence SGEGSFQPSQENPQ (SEQ ID NO: 93);

(k) the eleventh peptide comprises the amino acid sequence GQQGYYPTSPQQSG (SEQ ID NO: 94);

(l) the twelfth peptide comprises the amino acid sequence PEQPEQPFPEQPQQ (SEQ ID NO: 95);

(m) the thirteenth peptide comprises the amino acid sequence QPPFSEQEQPVLPQ (SEQ ID NO: 96);

(n) the fourteenth peptide comprises the amino acid sequence PEQPFPEQPIPEQPQPYP (SEQ ID NO: 97);

(o) the fifteenth peptide comprises the amino acid sequence QPYPQPELPYPQPQ (SEQ ID NO: 98);

(p) the sixteenth peptide comprises the amino acid sequence QPFPQPELPYPYPQ (SEQ ID NO: 99);

(q) the seventeenth peptide comprises the amino acid sequence PQEQPFPEQPIPEQP (SEQ ID NO: 100); and

(r) the eighteenth peptide comprises the amino acid sequence QPQPYPEQPQPFPQQ (SEQ ID NO: 101).

In some embodiments, the composition comprises (or consists of) the first, second, and third peptide. In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, tenth, eleventh, twelfth, thirteenth, fifteenth, seventeenth, and eighteenth peptides.

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 14 to 20 amino acids in length.

In some embodiments of any one of the compositions provided, a composition comprises (or consists of) any one of the peptide pools as described in the examples provided. In some embodiments, a composition comprising the epitopes of any one of the peptide pools of the examples is provided. In some embodiments of any one of the compositions, the peptides or epitopes are in equimolar amounts.

In some embodiments of any one of the compositions provided, each of the peptides are present in an amount of 2.5 ug/mL in the composition. In some embodiments of any one of the compositions provided, each of the peptides are present in an amount of 5 ug/mL in the composition. In some embodiments of any one of the compositions provided, each of the peptides are present in an amount of 10 ug/mL in the composition. In some embodiments of any one of the compositions provided, each of the peptides are present in an amount of 20 ug/mL in the composition. In some embodiments of any one of the compositions provided, each of the peptides are present in an amount of 25 ug/mL in the composition. In some embodiments of any one of the compositions provided, each of the peptides are present in an amount of 50 ug/mL in the composition. In some embodiments of any one of the compositions provided, each of the peptides are present in an amount of 5 uM (micromolar) in the composition. In some embodiments of any one of the compositions provided, each of the peptides are present in an amount of 10 uM in the composition. In some embodiments of any one of the compositions provided, each of the peptides are present in an amount of 25 uM in the composition. In some embodiments of any one of the compositions provided, each of the peptides are present in an amount of 50 uM in the composition.

In some embodiments, the composition comprises (or consists of) at least one (e.g., one, two, three, four, five, six, seven, eight, nine, ten, eleven, twelve, thirteen, fourteen, fifteen, or sixteen) of:

(i) a first peptide comprising the amino acid sequence PFPQPELPY (SEQ ID NO: 102) and the amino acid sequence PQPELPYPQ (SEQ ID NO: 103);

(ii) a second peptide comprising the amino acid sequence PFPQPEQPF (SEQ ID NO: 104) and the amino acid sequence PQPEQPFPW (SEQ ID NO: 105);

(iii) a third peptide comprising the amino acid sequence PIPEQPQPY (SEQ ID NO: 106);

(iv) a fourth peptide comprising the amino acid sequence PFPQPEQPIP (SEQ ID NO: 107) and the amino acid sequence EQPIPVQPE (SEQ ID NO: 108);

(v) a fifth peptide comprising the amino acid sequence PFPQPEQPTPI (SEQ ID NO: 109) and the amino acid sequence EQPTPIQPE (SEQ ID NO: 110);

(vi) a sixth peptide comprising the amino acid sequence PQPEQPFPL (SEQ ID NO: 111) and the amino acid sequence EQPFPLQPE (SEQ ID NO: 112);

(vii) a seventh peptide comprising the amino acid sequence PFPQPEQPF (SEQ ID NO: 113) and the amino acid sequence PQPEQPFSQ (SEQ ID NO: 114);

(viii) an eighth peptide comprising the amino acid sequence PYPEQPQPF (SEQ ID NO: 115);

(ix) a ninth peptide comprising the amino acid sequence PFPEQPEQIIP (SEQ ID NO: 116);

(x) a tenth peptide comprising the amino acid sequence EGSFQPSQE (SEQ ID NO: 117);

(xi) an eleventh peptide comprising the amino acid sequence QGYYPTSPQ (SEQ ID NO: 118);

(xii) a twelfth peptide comprising the amino acid sequence EQPEQPFPEQPQ (SEQ ID NO: 119);

(xiii) a thirteenth peptide comprising the amino acid sequence PFSEQEQPV (SEQ ID NO: 120);

(xiv) a fourteenth peptide comprising the amino acid sequence EQPFPEQPI (SEQ ID NO: 121) and PIPEQPQPY (SEQ ID NO: 122);

(xv) a fifteenth peptide comprising the amino acid sequence PQPELPYPQ (SEQ ID NO: 123) and the amino acid sequence PYPQPELPY (SEQ ID NO: 124);

(xvi) a sixteenth peptide comprising the amino acid sequence PFPQPELPY (SEQ ID NO: 125) and the amino acid sequence PQPELPYPY (SEQ ID NO: 126); and

(xvii) a seventeenth peptide comprising the amino acid sequence EQPFPEQPI (SEQ ID NO: 127).

In some embodiments, (i) the first peptide comprises the amino acid sequence LQPFPQPELPYPQPQ (SEQ ID NO: 128); (ii) the second peptide comprises the amino acid sequence QPFPQPEQPFPWQP (SEQ ID NO: 129); (iii) the third peptide comprises the amino acid sequence PEQPIPEQPQPYPQQ (SEQ ID NO: 130); (iv) the fourth peptide comprises the amino acid sequence QPFPQPEQPIPVQPEQS (SEQ ID NO: 131); (v) the fifth peptide comprises the amino acid sequence QPFPQPEQPTPIQPEQP (SEQ ID NO: 132); (vi) the sixth peptide comprises the amino acid sequence QPFPQPEQPFPLQPEQP (SEQ ID NO: 133); (vii) the seventh peptide comprises the amino acid sequence QPFPQPEQPFSQQ (SEQ ID NO: 134); (viii) the eighth peptide comprises the amino acid sequence PQPYPEQPQPFPQQ (SEQ ID NO: 135); (ix) the ninth peptide comprises the amino acid sequence QPFPEQPEQIIPQQP (SEQ ID NO: 136); (x) the tenth peptide comprises the amino acid sequence SGEGSFQPSQENPQ (SEQ ID NO: 137); (xi) the eleventh peptide comprises the amino acid sequence GQQGYYPTSPQQSG (SEQ ID NO: 138); (xii) the twelfth peptide comprises the amino acid sequence PEQPEQPFPEQPQQ (SEQ ID NO: 139); (xiii) the thirteenth peptide comprises the amino acid sequence QPPFSEQEQPVLPQ (SEQ ID NO: 140); (xiv) the fourteenth peptide comprises the amino acid sequence PEQPFPEQPIPEQPQPYP (SEQ ID NO: 141); (xv) the fifteenth peptide comprises the amino acid sequence QPYPQPELPYPQPQ (SEQ ID NO: 142); (xvi) the sixteenth peptide comprises the amino acid sequence QPFPQPELPYPYPQ (SEQ ID NO: 143); and (xvii) the seventeenth peptide comprises the amino acid sequence EQPFPEQPI (SEQ ID NO: 144).

In some embodiments, the composition comprises (or consists of) the first, second, and third peptide. In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides.

In some embodiments, at least one of the peptides comprises an N-terminal pyroglutamate and/or a C-terminal amide group. In some embodiments, each of the peptides comprises an N-terminal pyroglutamate and/or a C-terminal amide group. In some embodiments, each of the peptides is less than full-length gluten. In some embodiments, each of the peptides is independently between 8 to 50 amino acids in length. In some embodiments, each of the peptides is independently between 10 to 30 amino acids in length. In some embodiments, each of the peptides is independently between 14 to 20 amino acids in length.

In some embodiments of any one of the compositions provided, each of the peptides are present in an amount of 2.5 ug/mL in the composition. In some embodiments of any one of the compositions provided, each of the peptides are present in an amount of 5 ug/mL in the composition. In some embodiments of any one of the compositions provided, each of the peptides are present in an amount of 10 ug/mL in the composition. In some embodiments of any one of the compositions provided, each of the peptides are present in an amount of 20 ug/mL in the composition. In some embodiments of any one of the compositions provided, each of the peptides are present in an amount of 25 ug/mL in the composition. In some embodiments of any one of the compositions provided, each of the peptides are present in an amount of 50 ug/mL in the composition. In some embodiments of any one of the compositions provided, each of the peptides are present in an amount of 5 uM (micromolar) in the composition. In some embodiments of any one of the compositions provided, each of the peptides are present in an amount of 10 uM in the composition. In some embodiments of any one of the compositions provided, each of the peptides are present in an amount of 25 uM in the composition. In some embodiments of any one of the compositions provided, each of the peptides are present in an amount of 50 uM in the composition.

In some embodiments, the method further comprises treating the subject if identified as having or at risk of having Celiac disease or providing information to the subject about a treatment. In some embodiments, the method further comprises a step of recommending a gluten-free diet if the subject is identified as having or at risk of having Celiac disease or providing information to the subject about such a diet. In some embodiments, the method further comprises performing other testing. In some embodiments, the other testing comprises performing a serology assay, genotyping, and/or an intestinal biopsy.

In some embodiments, the subject is HLA-DQ2.5 positive, HLA-DQ2.2 positive and/or HLA-DQ8 positive. In some embodiments, the subject is HLA-DQ2.5 positive.

In some embodiments, the method further comprises administering a composition comprising wheat, rye, and/or barley, or a composition comprising a gluten peptide, to the subject at least once a day for one day. In some embodiments, the method further comprises administering a composition comprising wheat, rye, and/or barley to the subject at least once a day for two days.

In some embodiments, the subject has not undergone a gluten challenge within 1 week of the sample being obtained from the subject. In some embodiments, the subject has a level of IFN-gamma that is reduced or the same as a control level of IFN-gamma. In some embodiments, the level of IFN-gamma is not statically significantly higher than the control level of IFN-gamma. In some embodiments, the control level of IFN-gamma is 7.2 pg/mL. In some embodiments, the subject is on a diet that contains gluten.

In some embodiments of any one of the methods provided herein, the method further comprises recording the level(s), the result(s) of the assessing and/or the treatment, or suggestion for treatment, based on the assessing.

Other aspects of the disclosure relate to a kit comprising a binding partner for IL-2 and any one of the compositions provided, such as a composition comprising (or consisting of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 145) and the amino acid sequence PQPELPYPQ (SEQ ID NO: 146);

(b) a second peptide comprising the amino acid sequence PFPQPEQPF (SEQ ID NO: 147) and the amino acid sequence PQPEQPFPW (SEQ ID NO: 148);

(c) a third peptide comprising the amino acid sequence EQPIPEQPQ (SEQ ID NO: 149) and the amino acid sequence PIPEQPQPY (SEQ ID NO: 150);

(d) a fourth peptide comprising the amino acid sequence PFPQPEQPI (SEQ ID NO: 151), the amino acid sequence PQPEQPIPV (SEQ ID NO: 152), and the amino acid sequence EQPIPVQPE (SEQ ID NO: 153);

(e) a fifth peptide comprising the amino acid sequence PFPQPEQPT (SEQ ID NO: 154), the amino acid sequence PQPEQPTPI (SEQ ID NO: 155), and the amino acid sequence EQPTPIQPE (SEQ ID NO: 156);

(f) a sixth peptide comprising the amino acid sequence PFPQPEQPF (SEQ ID NO: 157), the amino acid sequence PQPEQPFPL (SEQ ID NO: 158), and the amino acid sequence EQPFPLQPE (SEQ ID NO: 159);

(g) a seventh peptide comprising the amino acid sequence PFPQPEQPF (SEQ ID NO: 160) and the amino acid sequence PQPEQPFSQ (SEQ ID NO: 161);

(h) an eighth peptide comprising the amino acid sequence PYPEQPQPF (SEQ ID NO: 162);

(i) a ninth peptide comprising the amino acid sequence PFPEQPEQI (SEQ ID NO: 163);

(j) a tenth peptide comprising the amino acid sequence EGSFQPSQE (SEQ ID NO: 164);

(k) an eleventh peptide comprising the amino acid sequence QGYYPTSPQ (SEQ ID NO: 165);

(l) a twelfth peptide comprising the amino acid sequence EQPEQPFPE (SEQ ID NO: 166) and the amino acid sequence EQPFPEQPQ (SEQ ID NO: 167);

(m) a thirteenth peptide comprising the amino acid sequence PFSEQEQPV (SEQ ID NO: 168);

(n) a fourteenth peptide comprising the amino acid sequence EQPFPEQPI (SEQ ID NO: 169), the amino acid sequence PFPEQPIPE (SEQ ID NO: 170), the amino acid sequence EQPIPEQPQ (SEQ ID NO: 171), and the amino acid sequence PIPEQPQPY (SEQ ID NO: 172);

(o) a fifteenth peptide comprising the amino acid sequence PQPELPYPQ (SEQ ID NO: 173) and the amino acid sequence PYPQPELPY (SEQ ID NO: 174);

(p) a sixteenth peptide comprising the amino acid sequence PFPQPELPY (SEQ ID NO: 175) and the amino acid sequence PQPELPYPY (SEQ ID NO: 176);

(q) a seventeenth peptide comprising the amino acid sequence EQPFPEQPI (SEQ ID NO: 177) and the amino acid sequence PFPEQPIPE (SEQ ID NO: 178); and

(r) an eighteenth peptide comprising the amino acid sequence PQPYPEQPQ (SEQ ID NO: 179) and the amino acid sequence PYPEQPQPF (SEQ ID NO: 180).

In some embodiments,

(a) the first peptide comprises the amino acid sequence LQPFPQPELPYPQPQ (SEQ ID NO: 181);

(b) the second peptide comprises the amino acid sequence QPFPQPEQPFPWQP (SEQ ID NO: 182);

(c) the third peptide comprises the amino acid sequence PEQPIPEQPQPYPQQ (SEQ ID NO: 183);

(d) the fourth peptide comprises the amino acid sequence QPFPQPEQPIPVQPEQS (SEQ ID NO: 184);

(e) the fifth peptide comprises the amino acid sequence QPFPQPEQPTPIQPEQP (SEQ ID NO: 185);

(f) the sixth peptide comprises the amino acid sequence QPFPQPEQPFPLQPEQP (SEQ ID NO: 186);

(g) the seventh peptide comprises the amino acid sequence QPFPQPEQPFSQQ (SEQ ID NO: 187);

(h) the eighth peptide comprises the amino acid sequence PQPYPEQPQPFPQQ (SEQ ID NO: 188);

(i) the ninth peptide comprises the amino acid sequence QPFPEQPEQIIPQQP (SEQ ID NO: 189);

(j) the tenth peptide comprises the amino acid sequence SGEGSFQPSQENPQ (SEQ ID NO: 190);

(k) the eleventh peptide comprises the amino acid sequence GQQGYYPTSPQQSG (SEQ ID NO: 191);

(l) the twelfth peptide comprises the amino acid sequence PEQPEQPFPEQPQQ (SEQ ID NO: 192);

(m) the thirteenth peptide comprises the amino acid sequence QPPFSEQEQPVLPQ (SEQ ID NO: 193);

(n) the fourteenth peptide comprises the amino acid sequence PEQPFPEQPIPEQPQPYP (SEQ ID NO: 194);

(o) the fifteenth peptide comprises the amino acid sequence QPYPQPELPYPQPQ (SEQ ID NO: 195);

(p) the sixteenth peptide comprises the amino acid sequence QPFPQPELPYPYPQ (SEQ ID NO: 196);

(q) the seventeenth peptide comprises the amino acid sequence PQEQPFPEQPIPEQP (SEQ ID NO: 197); and

(r) the eighteenth peptide comprises the amino acid sequence QPQPYPEQPQPFPQQ (SEQ ID NO: 198).

In some embodiments, the composition comprises (or consists of) the first, second, and third peptides.

In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 20 ug/mL in the composition. In some embodiments of any one of the kits provided, each of the peptides are present in an amount of 50 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments, the composition comprises (or consists of) the first, second, third, fourth, fifth, sixth, seventh, eighth, ninth, tenth, eleventh, twelfth, and thirteenth peptides or the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, tenth, eleventh, twelfth, thirteenth, fifteenth, seventeenth, and eighteenth peptides. In some embodiments of any one of the kits provided, each of the peptides are present in an amount of 2.5 ug/mL in the composition. In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 25 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments of any one of the kits provided, each of the peptides comprises an N-terminal pyroglutamate and/or a C-terminal amide group.

In some embodiments of any one of the kits provided, the kit further comprises a composition comprising wheat, rye, and/or barley, such a foodstuff. In some embodiments of any one of the kits provided, the kit further comprises a second binding partner for IP-10, such as a secondary antibody.

Other aspects of the disclosure relate to a kit comprising a binding partner for IL-2 and a composition comprising (or consisting of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 199) and the amino acid sequence PQPELPYPQ (SEQ ID NO: 200);

(b) a second peptide comprising the amino acid sequence PFPQPEQPF (SEQ ID NO: 201) and the amino acid sequence PQPEQPFPW (SEQ ID NO: 202);

(c) a third peptide comprising the amino acid sequence PIPEQPQPY (SEQ ID NO: 203);

(d) a fourth peptide comprising the amino acid sequence PFPQPEQPIP (SEQ ID NO: 204) and the amino acid sequence EQPIPVQPE (SEQ ID NO: 205);

(e) a fifth peptide comprising the amino acid sequence PFPQPEQPTPI (SEQ ID NO: 206) and the amino acid sequence EQPTPIQPE (SEQ ID NO: 207);

(f) a sixth peptide comprising the amino acid sequence PQPEQPFPL (SEQ ID NO: 208) and the amino acid sequence EQPFPLQPE (SEQ ID NO: 209);

(g) a seventh peptide comprising the amino acid sequence PFPQPEQPF (SEQ ID NO: 210) and the amino acid sequence PQPEQPFSQ (SEQ ID NO: 211);

(h) an eighth peptide comprising the amino acid sequence PYPEQPQPF (SEQ ID NO: 212);

(i) a ninth peptide comprising the amino acid sequence PFPEQPEQIIP (SEQ ID NO: 213);

(j) a tenth peptide comprising the amino acid sequence EGSFQPSQE (SEQ ID NO: 214);

(k) an eleventh peptide comprising the amino acid sequence QGYYPTSPQ (SEQ ID NO: 215);

(l) a twelfth peptide comprising the amino acid sequence EQPEQPFPEQPQ (SEQ ID NO: 216);

(m) a thirteenth peptide comprising the amino acid sequence PFSEQEQPV (SEQ ID NO: 217);

(n) a fourteenth peptide comprising the amino acid sequence EQPFPEQPI (SEQ ID NO: 218) and PIPEQPQPY (SEQ ID NO: 219);

(o) a fifteenth peptide comprising the amino acid sequence PQPELPYPQ (SEQ ID NO: 220) and the amino acid sequence PYPQPELPY (SEQ ID NO: 221);

(p) a sixteenth peptide comprising the amino acid sequence PFPQPELPY (SEQ ID NO: 222) and the amino acid sequence PQPELPYPY (SEQ ID NO: 223); and

(q) a seventeenth peptide comprising the amino acid sequence EQPFPEQPI (SEQ ID NO: 224).

In some embodiments, (a) the first peptide comprises the amino acid sequence LQPFPQPELPYPQPQ (SEQ ID NO: 225); (b) the second peptide comprises the amino acid sequence QPFPQPEQPFPWQP (SEQ ID NO: 226); (c) the third peptide comprises the amino acid sequence PEQPIPEQPQPYPQQ (SEQ ID NO: 227); (d) the fourth peptide comprises the amino acid sequence QPFPQPEQPIPVQPEQS (SEQ ID NO: 228); (e) the fifth peptide comprises the amino acid sequence QPFPQPEQPTPIQPEQP (SEQ ID NO: 229); (f) the sixth peptide comprises the amino acid sequence QPFPQPEQPFPLQPEQP (SEQ ID NO: 230); (g) the seventh peptide comprises the amino acid sequence QPFPQPEQPFSQQ (SEQ ID NO: 231); (h) the eighth peptide comprises the amino acid sequence PQPYPEQPQPFPQQ (SEQ ID NO: 232); (i) the ninth peptide comprises the amino acid sequence QPFPEQPEQIIPQQP (SEQ ID NO: 233); (j) the tenth peptide comprises the amino acid sequence SGEGSFQPSQENPQ (SEQ ID NO: 234); (k) the eleventh peptide comprises the amino acid sequence GQQGYYPTSPQQSG (SEQ ID NO: 235); (l) the twelfth peptide comprises the amino acid sequence PEQPEQPFPEQPQQ (SEQ ID NO: 236); (m) the thirteenth peptide comprises the amino acid sequence QPPFSEQEQPVLPQ (SEQ ID NO: 237); (n) the fourteenth peptide comprises the amino acid sequence PEQPFPEQPIPEQPQPYP (SEQ ID NO: 238); (o) the fifteenth peptide comprises the amino acid sequence QPYPQPELPYPQPQ (SEQ ID NO: 239); (p) the sixteenth peptide comprises the amino acid sequence QPFPQPELPYPYPQ (SEQ ID NO: 240); and (q) the seventeenth peptide comprises the amino acid sequence EQPFPEQPI (SEQ ID NO: 241).

In some embodiments, the composition comprises (or consists of) the first, second, and third peptides.

In some embodiments, the composition comprises (or consists of) the first, second, third, fourth, fifth, sixth, seventh, eighth, ninth, tenth, eleventh, twelfth, and thirteenth peptides or the composition comprises (or consists of) the second, fourth, fifth, sixth, seventh, eighth, ninth, tenth, eleventh, twelfth, thirteenth, fourteenth, fifteenth, and sixteenth peptides.

In some embodiments, each of the peptides comprises an N-terminal pyroglutamate and/or a C-terminal amide group.

In some embodiments of any one of the kits provided, the composition comprises at least one (e.g., one, two, three, four, five, six, seven, eight, nine, ten, eleven, twelve, thirteen, fourteen, fifteen, or sixteen) peptide(s), the at least one peptide comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 242), PQPELPYPQ (SEQ ID NO: 243), PFPQPEQPF (SEQ ID NO: 244), PQPEQPFPW (SEQ ID NO: 245), EQPIPEQPQ (SEQ ID NO: 246), PIPEQPQPY (SEQ ID NO: 247), PFPQPEQPI (SEQ ID NO: 248), PQPEQPIPV (SEQ ID NO: 249), EQPIPVQPE (SEQ ID NO: 250), PFPQPEQPT (SEQ ID NO: 251), PQPEQPTPI (SEQ ID NO: 252), EQPTPIQPE (SEQ ID NO: 253), PQPEQPFPL (SEQ ID NO: 254), EQPFPLQPE (SEQ ID NO: 255), EGSFQPSQE (SEQ ID NO: 256), QGYYPTSPQ (SEQ ID NO: 257), EQPEQPFPE (SEQ ID NO: 258), PFSEQEQPV (SEQ ID NO: 259), PYPQPELPY (SEQ ID NO: 260), EQPFPEQPI (SEQ ID NO: 261), PFPEQPIPE (SEQ ID NO: 262), PYPEQPQPF (SEQ ID NO: 263), and PQPYPEQPQ (SEQ ID NO: 264). In some embodiments, the composition comprises (or consists of) at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 265), PQPELPYPQ (SEQ ID NO: 266), PFPQPEQPF (SEQ ID NO: 267), PQPEQPFPW (SEQ ID NO: 268), EQPIPEQPQ (SEQ ID NO: 269), PIPEQPQPY (SEQ ID NO: 270), PFPQPEQPI (SEQ ID NO: 271), PQPEQPIPV (SEQ ID NO: 272), EQPIPVQPE (SEQ ID NO: 273), PFPQPEQPT (SEQ ID NO: 274), PQPEQPTPI (SEQ ID NO: 275), EQPTPIQPE (SEQ ID NO: 276), PQPEQPFPL (SEQ ID NO: 277), EQPFPLQPE (SEQ ID NO: 278), EGSFQPSQE (SEQ ID NO: 279), QGYYPTSPQ (SEQ ID NO: 280), EQPEQPFPE (SEQ ID NO: 281), PFSEQEQPV (SEQ ID NO: 282), PYPQPELPY (SEQ ID NO: 283), EQPFPEQPI (SEQ ID NO: 284), PFPEQPIPE (SEQ ID NO: 285), PYPEQPQPF (SEQ ID NO: 286), and PQPYPEQPQ (SEQ ID NO: 287).

In some embodiments of any one of the kits provided, the composition comprises (or consists of) at least one (e.g., one, two, three, four, five, six, seven, eight, nine, ten, eleven, twelve, thirteen, fourteen, fifteen, or sixteen) of:

(a) a first peptide comprising the amino acid sequence PFPQPELPY (SEQ ID NO: 288) and the amino acid sequence PQPELPYPQ (SEQ ID NO: 289);

(b) a second peptide comprising the amino acid sequence PFPQPEQPF (SEQ ID NO: 290) and the amino acid sequence PQPEQPFPW (SEQ ID NO: 291);

(c) a third peptide comprising the amino acid sequence EQPIPEQPQ (SEQ ID NO: 292) and the amino acid sequence PIPEQPQPY (SEQ ID NO: 293);

(d) a fourth peptide comprising the amino acid sequence PFPQPEQPI (SEQ ID NO: 294) and the amino acid sequence PQPEQPIPV (SEQ ID NO: 295);

(e) a fifth peptide comprising the amino acid sequence EQPIPVQPE (SEQ ID NO: 296);

(f) a sixth peptide comprising the amino acid sequence PFPQPEQPT (SEQ ID NO: 297) and the amino acid sequence PQPEQPTPI (SEQ ID NO: 298);

(g) a seventh peptide comprising the amino acid sequence EQPTPIQPE (SEQ ID NO: 299);

(h) an eighth peptide comprising the amino acid sequence PQPEQPFPL (SEQ ID NO: 300);

(i) a ninth peptide comprising the amino acid sequence EQPFPLQPE (SEQ ID NO: 301);

(j) a tenth peptide comprising the amino acid sequence EGSFQPSQE (SEQ ID NO: 302);

(k) a eleventh peptide comprising the amino acid sequence QGYYPTSPQ (SEQ ID NO: 303);

(l) a twelfth peptide comprising the amino acid sequence EQPEQPFPE (SEQ ID NO: 304);

(m) a thirteenth peptide comprising the amino acid sequence PFSEQEQPV (SEQ ID NO: 305);

(n) a fourteenth peptide comprising the amino acid sequence PYPQPELPY (SEQ ID NO: 306);

(o) a fifteenth peptide comprising the amino acid sequence EQPFPEQPI (SEQ ID NO: 307) and the amino acid sequence PFPEQPIPE (SEQ ID NO: 308); and

(p) a sixteenth peptide comprising the amino acid sequence PYPEQPQPF (SEQ ID NO: 309) and the amino acid sequence PQPYPEQPQ (SEQ ID NO: 310).

In some embodiments of any one of the kits provided,

(a) the first peptide comprises the amino acid sequence PFPQPELPYPQP (SEQ ID NO: 311);

(b) the second peptide comprises the amino acid sequence PFPQPEQPFPWQ (SEQ ID NO: 312);

(c) the third peptide comprises the amino acid sequence EQPIPEQPQPYP (SEQ ID NO: 313);

(d) the fourth peptide comprises the amino acid sequence PFPQPEQPIPVQ (SEQ ID NO: 314);

(e) the fifth peptide comprises the amino acid sequence PEQPIPVQPEQS (SEQ ID NO: 315);

(f) the sixth peptide comprises the amino acid sequence PFPQPEQPTPIQ (SEQ ID NO: 316);

(g) the seventh peptide comprises the amino acid sequence PEQPTPIQPEQP (SEQ ID NO: 317);

(h) the eighth peptide comprises the amino acid sequence PFPQPEQPFPLQ (SEQ ID NO: 318);

(i) the ninth peptide comprises the amino acid sequence PEQPFPLQPEQP (SEQ ID NO: 319);

(j) the tenth peptide comprises the amino acid sequence GEGSFQPSQENP (SEQ ID NO: 320);

(k) the eleventh peptide comprises the amino acid sequence QQGYYPTSPQQS (SEQ ID NO: 321);

(l) the twelfth peptide comprises the amino acid sequence PEQPEQPFPEQP (SEQ ID NO: 322);

(m) the thirteenth peptide comprises the amino acid sequence PPFSEQEQPVLP (SEQ ID NO: 323);

(n) the fourteenth peptide comprises the amino acid sequence PYPQPELPYPQP (SEQ ID NO: 324);

(o) the fifteenth peptide comprises the amino acid sequence EQPFPEQPIPEQ (SEQ ID NO: 325); and

(p) the sixteenth peptide comprises the amino acid sequence PQPYPEQPQPFP (SEQ ID NO: 326).

In some embodiments of any one of the kits provided, the composition comprises (or consists of) at least four (e.g., four, five, six, seven, eight, nine, ten, eleven, twelve, thirteen, fourteen, fifteen or sixteen) of the peptides. In some embodiments of any one of the kits provided, the composition comprises (or consists of) the peptides in (a)-(p).

In some embodiments of any one of the kits provided, at least one of the peptides comprises an N-terminal pyroglutamate and/or a C-terminal amide group. In some embodiments of any one of the kits provided, each of the peptides comprises an N-terminal pyroglutamate and/or a C-terminal amide group. In some embodiments of any one of the kits provided, each of the peptides is less than full-length gluten. In some embodiments of any one of the kits provided, each of the peptides is independently between 8 to 50 amino acids in length. In some embodiments, each of the peptides is independently between 10 to 30 amino acids in length. In some embodiments, each of the peptides is independently between 12 to 30 amino acids in length. In some embodiments, each of the peptides is 13 amino acids in length.

In some embodiments of any one of the kits provided, each of the peptides are present in an amount of 2.5 ug/mL in the composition. In some embodiments of any one of the kits provided, each of the peptides are present in an amount of 5 ug/mL in the composition. In some embodiments of any one of the kits provided, each of the peptides are present in an amount of 10 ug/mL in the composition. In some embodiments of any one of the kits provided, each of the peptides are present in an amount of 20 ug/mL in the composition. In some embodiments of any one of the kits provided, each of the peptides are present in an amount of 25 ug/mL in the composition. In some embodiments of any one of the kits provided, each of the peptides are present in an amount of 50 ug/mL in the composition. In some embodiments of any one of the kits provided, each of the peptides are present in an amount of 5 uM (micromolar) in the composition. In some embodiments of any one of the kits provided, each of the peptides are present in an amount of 10 uM in the composition. In some embodiments of any one of the kits provided, each of the peptides are present in an amount of 25 uM in the composition. In some embodiments of any one of the kits provided, each of the peptides are present in an amount of 50 uM in the composition.

In some embodiments of any one of the kits provided, the kit further comprises a composition comprising wheat, rye, and/or barley, such a foodstuff. In some embodiments of any one of the kits provided, the kit further comprises a second binding partner for IP-10, such as a secondary antibody.

In some embodiments of any one of the compositions, methods or kits provided, the peptides in a composition each consist of the recited amino acid sequence(s).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is two graphs showing exemplary net IL-2 release and relative IL-2 release in whole blood contacted with Peptide Pool 1 compared to whole blood contacted with PBS control (NIL). The whole blood was collected before (Day 0) and after (Day 6) 3-day oral gluten challenge.

FIG. 2 is a table showing IFNγ and IL-2 MAGPIX data in blood samples from subjects with Celiac disease after oral gluten challenge prior to a first treatment dose and after a last treatment dose with a peptide composition. The blood samples were contacted with a peptide composition or buffered solution (NIL) and the level IFNγ and IL-2 was measured by MAGPIX.

FIG. 3 is a table showing IFNγ and IL-2 MAGPIX data in blood samples from subjects with Celiac disease after oral gluten challenge prior to a first treatment dose and after a last treatment dose with a placebo. The blood samples were contacted with a peptide composition or buffered solution (NIL) and the level IFNγ and IL-2 was measured by MAGPIX.

### DETAILED DESCRIPTION

Celiac disease (CD, also sometimes referred to as cœliac disease, c(o)eliac sprue, non-tropical sprue, endemic sprue, gluten enteropathy or gluten-sensitive enteropathy, and gluten intolerance) is an autoimmune disorder of the small intestine caused by ingestion of gluten-containing foods that occurs in people of all ages, ranging from middle infancy onward, and affects approximately 1% of people in Europe and North America. Untreated Celiac disease is associated with increased risk of adenocarcinoma (small intestine cancer) and lymphoma of the small bowel (enteropathy-associated T-cell lymphoma), as well as other complications, such as ulcerative jejunitis (ulcer formation of the small bowel) and stricturing (narrowing as a result of scarring with obstruction of the bowel). In many of those affected, Celiac disease is unrecognized, but this clinical oversight is now being rectified with greater clinical awareness.

Celiac disease generally occurs in genetically susceptible individuals who possess either HLA-DQ2 encoded by HLA-DQA1*05 and HLA-DQB1*02 (accounting for about 90% of individuals), variants of HLA-DQ2, or HLA-DQ8. Without wishing to be bound by theory, such individuals are thought to mount an inappropriate HLA-DQ2- and/or DQ8-restricted CD4⁺ T cell-mediated immune response to peptides derived from the aqueous-insoluble proteins of wheat flour, gluten, and related proteins in rye and barley (herein referred to as gluten peptides).

Celiac disease is diagnosed by small bowel biopsy showing villous atrophy, crypt hyperplasia and raised intra-epithelial lymphocytes, and supported by the presence of celiac disease-specific serology (IgA specific for transglutaminase and/or IgA and IgG specific for deamidated gliadin peptide). Intestinal histology and serological abnormalities normalize or improve within weeks to months of adopting gluten-free diet. In general, celiac disease can be excluded if certain alleles encoding HLA-DQA1*05, DQB1*02 and DQB1*0302 are not present. In patients who have adopted a gluten-free diet (GFD) without definitive diagnosis, reintroduction of gluten into the diet has been necessary to make a firm diagnosis of celiac disease. Reintroduction of >3 g/day gluten (about 1.5 slices of wheat bread) daily leads to intestinal tissue damage in the majority of patients with celiac disease usually strictly adherent to gluten free diet.

Small bowel biopsy typically requires an endoscopy, which is inconvenient and may be inconclusive if biopsies are not performed at multiple sites in the duodenum, processed meticulously and interpreted correctly. Requiring small bowel biopsy may also delay treatment because of the importance of continuing to consume gluten until after the procedure. Furthermore, celiac disease cannot be diagnosed in patients who have excluded gluten from their diet if serology and histology do show typical diagnostic features.

Oral gluten challenge for 3 days mobilizes gluten-reactive T cells that can generally be measured six days after commencing the challenge. However, patients may not tolerate consuming gluten for three days and results are not available for a number of days.

Accordingly, aspects of the disclosure relate to methods of identifying a subject having or at risk for having celiac disease by determining a level of IL-2 in a sample from the subject.

### Methods

One aspect of the disclosure relates to methods useful for diagnosis of a subject, such as a subject having or suspected of having Celiac disease. The methods involve determining (e.g., measuring) a level of IL-2 in a sample from the subject.

In some embodiments, the method comprises determining a level of IL-2 in a sample comprising a T cell from a subject having or suspected of having Celiac disease, wherein the sample has been contacted (e.g., contacted ex vivo) with a composition comprising at least one gluten peptide as described herein prior to the determining; and assessing whether or not the subject has or is at risk of having Celiac disease.

In some embodiments, the determining step comprises contacting (e.g., contacting ex vivo) the sample comprising the T cell with the composition comprising at least one gluten peptide; and measuring the level of IL-2 in the sample comprising the T cell after the contacting. Methods for measuring the level of IL-2 are described herein.

In some embodiments, any one of the methods further comprises comparing the level of IL-2 in the sample with a control level of IL-2. The comparing may be accomplished with the assistance of a software program on a computer. In some embodiments, the comparing comprises a statistical analysis, such as a paired T-test.

In some embodiments, assessing comprises identifying the subject as having or at risk of having Celiac disease if the IL-2 level is elevated compared to a control level of IL-2; or not having or not at risk of having Celiac disease if the IL-2 level is reduced compared to the control level of IL-2 or the same as the control level of IL-2. Control levels are further described herein. In some embodiments, assessing comprises identifying the subject as having or at risk of having Celiac disease if the IL-2 level is at least 3 pg/mL above a control level of IL-2; or not having or not at risk of having Celiac disease if the IL-2 level is less than 3 pg/mL above a control level of IL-2 (e.g. whole blood incubated with a composition described herein comprising at least one gluten peptide in phosphate buffered saline (PBS) compared to PBS alone). In some embodiments, assessing comprises identifying the subject as having or at risk of having Celiac disease if the IL-2 level is at least 40% greater a control level of IL-2; or not having or not at risk of having Celiac disease if the IL-2 level is less than 40% greater than a control level of IL-2. In some embodiments, assessing comprises identifying the subject as having or at risk of having Celiac disease if the IL-2 level is at least 40% greater than and at least 3 pg/mL above a control level of IL-2; or not having or not at risk of having Celiac disease if the IL-2 level is less than 40% greater than and is less than 3 pg/mL above a control level of IL-2.

In some embodiments, assessing comprises identifying the subject as having or at risk of having Celiac disease if the IL-2 level is at least 3 pg/mL above a control level of IL-2 and a stimulation index that is greater than 1.4; or not having or not at risk of having Celiac disease if the IL-2 level is less than 3 pg/mL above a control level of IL-2 and a stimulation index that is less than or equal to 1.4.

In some embodiments of any one of the methods provided herein, the method further comprising treating or suggesting a treatment if the subject is identified as having or likely of having celiac disease. In some embodiments of any one of the methods provided herein, the method further comprises recommending a gluten-free diet and/or providing information in regard thereto to the subject. In some embodiments of any one of the methods provided herein, the method further comprises administering a treatment, or providing information in regard thereto, to the subject. Suitable treatments are described herein. In some embodiments, the treatment is a composition comprising a gluten peptide as described herein. In some embodiments, the treatment comprises a gluten-free diet.

In some embodiments, any one of the methods described herein further comprises recording whether or not the subject has Celiac disease based on the assessing. In some embodiments, any one of the methods described herein further comprises transmitting, such as to a database, whether or not the subject has celiac disease based on the assessing. The transmitting may be accomplished, e.g., via a computer or network of computers.

### IL-2 and Measuring IL-2 Levels

Interleukin-2 (IL-2) is a protein that in humans is encoded by the IL2 gene. IL-2 is a secreted cytokine and binds, e.g., to the heterotrimeric protein receptor interleukin-2 receptor (IL-2R). The Genbank ID number for the human IL2 gene is 3558. Exemplary mRNA sequences and protein sequences for IL-2 are shown below.

>gi|125661059|ref|NM_000586.3| Homo sapiens
interleukin 2 (IL2), mRNA
(SEQ ID NO: 327)
AGTTCCCTATCACTCTCTTTAATCACTACTCACAGTAACCTCAACTCCTG
CCACAATGTACAGGATGCAACTCCTGTCTTGCATTGCACTAAGTCTTGCA
CTTGTCACAAACAGTGCACCTACTTCAAGTTCTACAAAGAAAACACAGCT
ACAACTGGAGCATTTACTGCTGGATTTACAGATGATTTTGAATGGAATTA
ATAATTACAAGAATCCCAAACTCACCAGGATGCTCACATTTAAGTTTTAC
ATGCCCAAGAAGGCCACAGAACTGAAACATCTTCAGTGTCTAGAAGAAGA
ACTCAAACCTCTGGAGGAAGTGCTAAATTTAGCTCAAAGCAAAAACTTTC
ACTTAAGACCCAGGGACTTAATCAGCAATATCAACGTAATAGTTCTGGAA
CTAAAGGGATCTGAAACAACATTCATGTGTGAATATGCTGATGAGACAGC
AACCATTGTAGAATTTCTGAACAGATGGATTACCTTTTGTCAAAGCATCA
TCTCAACACTGACTTGATAATTAAGTGCTTCCCACTTAAAACATATCAGG
CCTTCTATTTATTTAAATATTTAAATTTTATATTTATTGTTGAATGTATG
GTTTGCTACCTATTGTAACTATTATTCTTAATCTTAAAACTATAAATATG
GATCTTTTATGATTCTTTTTGTAAGCCCTAGGGGCTCTAAAATGGTTTCA
CTTATTTATCCCAAAATATTTATTATTATGTTGAATGTTAAATATAGTAT
CTATGTAGATTGGTTAGTAAAACTATTTAATAAATTTGATAAATATAAAA
AAAAAAAAAAAAAAAAAAAAAA
>gi|28178861|ref|NP_000577.2| interleukin-2
precursor [Homo sapiens]
(SEQ ID NO: 328)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT
>gi|28178861|ref|NP_000577.2| interleukin-2
mature protein [Homo sapiens]
(SEQ ID NO: 329)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

Aspects of the disclosure relate to methods that comprise determining or measuring a level of IL-2 in a sample comprising a T cell from a subject, such as a subject suspected of having Celiac disease. Such methods are described herein.

Measuring an IL-2 level can be accomplished using any assay known in the art (see, e.g., Molecular Cloning: A Laboratory Manual, M. Green and J. Sambrook, Fourth Edition, Cold Spring Harbor Laboratory Press, Cold Spring Harbor, N.Y., 2012, Current Protocols in Molecular Biology, F. M. Ausubel, et al., eds., John Wiley & Sons, Inc., New York). Levels of IL-2 include levels of IL-2 mRNA and/or levels of IL-2 protein. In a preferred embodiment, levels of IL-2 are protein levels.

Assays for detecting IL-2 mRNA include, but are not limited to, Northern blot analysis, RT-PCR, sequencing technology, RNA in situ hybridization (using e.g., DNA or RNA probes to hybridize to RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer) with addressable locations, such as an Affymetrix microarray (Affymetrix®, Santa Clara, Calif.)). Methods for designing nucleic acid binding partners, such as probes, are well known in the art. In some embodiments, the nucleic acid binding partners bind to a part of or an entire nucleic acid sequence of IL-2, such as a sequence provided herein.

Assays for detecting protein levels include, but are not limited to, immunoassays (also referred to herein as immune-based or immuno-based assays, e.g., Western blot, ELISA, and ELISPOT assays), Mass spectrometry, and multiplex bead-based assays. Binding partners for protein detection can be designed using methods known in the art and as described herein. In some embodiments, the IL-2 protein binding partners, e.g., anti-IL-2 antibodies, bind to a part of or an entire amino acid sequence of IL-2, such as an IL-2 protein sequence provided herein. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

In some embodiments, measuring a level of IL-2 comprises a multiplex bead-based assay. An exemplary multiplex bead-based assay involves use of magnetic beads that are internally dyed with fluorescent dyes to produce a specific spectral address. Binding partners (e.g., antibodies) are conjugated to the surface of beads to capture IL-2. The sample is loaded into a 96-well plate containing the beads and the sample is incubated to allow binding of IL-2 to the beads. A second biotinylated binding partner for IL-2 is added after the IL-2 binds to the beads. A streptavidin-conjugated detectable label is then bound to the biotin. Light emitting diodes are used to illuminate the samples, causing the fluorescent dyes in the beads to fluoresce, as well as the detectable label to fluoresce. The concentration of IL-2 is then determined based on the level of fluorescence. An exemplary system for running a multiplex bead-based assay is the MAGPIX® system available from Luminex® Corporation (see, e.g., U.S. Pat. No. 8,031,918, U.S. Pat. No. 8,296,088, U.S. Pat. No. 8,274,656, U.S. Pat. No. 8,532,351, U.S. Pat. No. 8,542,897, U.S. Pat. No. 6,514,295, U.S. Pat. No. 6,599,331, U.S. Pat. No. 6,632,526, U.S. Pat. No. 6,929,859, U.S. Pat. No. 7,445,844, U.S. Pat. No. 7,718,262, U.S. Pat. No. 8,283,037, and U.S. Pat. No. 8,568,881, all of which are incorporated by reference herein, and in particular the systems provided herein).

In some embodiments, measuring a level of IL-2 comprises an enzyme-linked immunosorbent assay (ELISA) or enzyme-linked immunosorbent spot (ELISpot) assay. ELISA and ELISpot assays are well known in the art (see, e.g., U.S. Pat. Nos. 5,939,281, 6,410,252, and 7,575,870; Czerkinsky C, Nilsson L, Nygren H, Ouchterlony O, Tarkowski A (1983) “A solid-phase enzyme-linked immunospot (ELISPOT) assay for enumeration of specific antibody-secreting cells”. J Immunol Methods 65 (1-2): 109-121 and Lequin R (2005). “Enzyme immunoassay (EIA)/enzyme-linked immunosorbent assay (ELISA)”. Clin. Chem. 51 (12): 2415-8).

An exemplary ELISpot assay involves a binding agent for IL-2 (e.g., an anti-IL-2 antibody) that is coated aseptically onto a PVDF (polyvinylidene fluoride)-backed microplate. Cells of interest (e.g., peripheral blood mononuclear cells) are plated out at varying densities, along with a peptide as described herein, and allowed to incubate for a period of time (e.g., about 24 hours). The at least one cytokine secreted by activated cells is captured locally by the binding partner for the at least one cytokine on the high surface area PVDF membrane. After the IL-2 is immobilized, a second binding partner for IL-2 is added, forming a complex with the immobilized at least one cytokine. The binding partner can be linked to a detectable label (e.g., a fluorophor or an enzyme), or can itself be detected by an agent that recognizes the binding partner for the at least one cytokine (e.g., a secondary antibody) that is linked to a detectable label (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

An exemplary ELISA involves at least one binding partner, e.g., an antibody or antigen-binding fragment thereof, with specificity for a particular antigen, such as IL-2. The sample with an unknown amount of antigen can be immobilized on a solid support (e.g., a polystyrene microtiter plate) either non-specifically (via adsorption to the surface) or specifically (via capture by another binding partner specific to the same antigen, as in a “sandwich” ELISA). After the antigen is immobilized, the binding partner for IL-2 is added, forming a complex with the immobilized IL-2. The binding partner can be attached to a detectable label as described herein (e.g., a fluorophor or an enzyme), or can itself be detected by an agent that recognizes the IL-2 binding partner that is attached to a detectable label as described herein (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

In some embodiments, a level of IL-2 is measured using an ELISA similar to the QuantiFERON®-TB Gold IT test (Cellestis Inc., Valencia, Calif.) for detecting mycobacterium, except wherein the TB antigen is replaced with at least one gluten peptide as described herein and IL-2 is detected in place of IFN-γ. The ELISA in the context of TB antigen has been described (see, e.g., U.S. Pat. Nos. 5,494,799, 5,334,504, and 7,608,382). As an exemplary method, at least one gluten peptide as defined herein is dried onto the inner wall of a blood collection tube. A negative control tube containing no antigen is provided. A positive control tube containing a mitogen is also provided. Blood from a subject is drawn into each of the three tubes. Each tube is agitated to ensure mixing. The tubes are then incubated at 37 degrees Celsius, preferably immediately after blood draw or at least within about 16 hours of collection. After incubation, the cells are separated from the plasma by centrifugation. The plasma is then loaded into an ELISA plate for detection of levels of IL-2 present in the plasma. A standard ELISA assay as described above can then be used to detect the levels of IL-2 present in each plasma sample.

In some embodiments, the level of IL-2 detected using any one of the methods above or any other appropriate method is then compared to a control level of IL-2 as described herein. In some embodiments, the control level is measured using any one of the methods above or any other as appropriate. In some embodiments, the same method is used to detect the level of the IL-2 in the sample of the subject and in the control level of IL-2.

### Samples

Samples, as used herein, refer to biological samples taken or derived from a subject, e.g., a subject having or suspected of having Celiac disease. Examples of samples include tissue samples or fluid samples. Examples of fluid samples are blood, plasma, and serum. In some embodiments, the sample comprises a T cell. In some embodiments, the sample comprises a T cell and a leukocyte, such as a monocyte or granulocyte. In some embodiments, the sample comprises a T cell and monocyte or granulocyte. In some embodiments, the sample comprises a T cell, a monocyte and a granulocyte. Different types of leukocytes can be identified using methods known in the art, e.g., using a Hematoxylin and eosin stain and/or antibodies specific for different types of leukocytes. In some embodiments, the sample comprises whole blood or peripheral blood mononuclear cells (PBMCs). Whole blood includes blood cells (such as erythrocytes, leukocytes, and platelets) and plasma, and may optionally include additives such as anti-coagulants. PBMCs include singly-nucleated blood cells (such as lymphocytes, monocytes, and macrophages) isolated from whole blood, e.g., using Ficoll or other methods known in the art. T cells include CD8⁺ and/or CD4⁺ T cells. The T cell may be, e.g., a gluten-reactive CD4⁺ T cell.

In some embodiments, any one of the methods described herein comprises obtaining the sample from the subject. In some embodiments, a first and second sample are contemplated. “First” and “second” are not meant to imply an order of use or an order in which the samples are obtained, unless specifically stated otherwise. In some embodiments, the second sample is a control sample to be used to obtain a control IL-2 level (controls and control levels are discussed herein). In some embodiments, the first sample and/or second sample are obtained from the subject prior to, during, or after a gluten challenge as described herein. In some embodiments, the first sample is obtained from the subject after a gluten challenge. In some embodiments, the second sample is obtained from the subject prior to a gluten challenge. Additional samples, e.g., third, fourth, fifth, etc., are also contemplated if additional measurements of IL-2 levels are desired.

### Subjects

A subject may include any subject that is suspected of having Celiac disease. Preferably, the subject is a human. In some embodiments, the subject has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). In some embodiments, the subject is HLA-DQ2.5 positive (i.e., has both susceptibility alleles DQA1*05 and DQB1*02). In some embodiments, the subject is HLA-DQ2.2 positive (i.e., has both susceptibility alleles DQA1*02 and DQB1*02). In some embodiments, the subject is HLA-DQ8 positive (i.e., has both susceptibility alleles DQA1*03 and DQB1*0302). In some embodiments, the subject is HLA-DQ2.2 positive and HLA-DQ2.5 positive. In some embodiments, the subject is HLA-DQ8 positive and HLA-DQ2.5 positive. In some embodiments, the subject is HLA-DQ2.2 positive and HLA-DQ8 positive. In some embodiments, a subject may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes. In some embodiments of any one of the methods provided herein, the subject is on a gluten-free diet. In some embodiments of any one of the methods provided herein, the subject is on a diet that contains gluten.

In some embodiments of any one of the methods provided herein, the subject has a level of IFN-γ that is reduced or the same as a control level of IFN-γ. In some embodiments of any one of the methods provided herein, the subject's level of IFN-γ is such that a clinician may expect little to no risk of Celiac disease for the subject. In some embodiments, the level of IFN-γ is such that a clinician would expect additional testing to be needed for a more assured diagnosis. A level of IFN-γ may be measured using any method known in the art or described herein (e.g., ELISA, ELISpot, or multiplex bead-based assay). The level may be a RNA level or a protein level. Exemplary RNA and protein sequences of IFN-γ are provided below.

>gi|56786137|ref|NM_000619.2| Homo sapiens
interferon, gamma (IFNG), mRNA
(SEQ ID NO: 330)
CACATTGTTCTGATCATCTGAAGATCAGCTATTAGAAGAGAAAGATCAGT
TAAGTCCTTTGGACCTGATCAGCTTGATACAAGAACTACTGATTTCAACT
TCTTTGGCTTAATTCTCTCGGAAACGATGAAATATACAAGTTATATCTTG
GCTTTTCAGCTCTGCATCGTTTTGGGTTCTCTTGGCTGTTACTGCCAGGA
CCCATATGTAAAAGAAGCAGAAAACCTTAAGAAATATTTTAATGCAGGTC
ATTCAGATGTAGCGGATAATGGAACTCTTTTCTTAGGCATTTTGAAGAAT
TGGAAAGAGGAGAGTGACAGAAAAATAATGCAGAGCCAAATTGTCTCCTT
TTACTTCAAACTTTTTAAAAACTTTAAAGATGACCAGAGCATCCAAAAGA
GTGTGGAGACCATCAAGGAAGACATGAATGTCAAGTTTTTCAATAGCAAC
AAAAAGAAACGAGATGACTTCGAAAAGCTGACTAATTATTCGGTAACTGA
CTTGAATGTCCAACGCAAAGCAATACATGAACTCATCCAAGTGATGGCTG
AACTGTCGCCAGCAGCTAAAACAGGGAAGCGAAAAAGGAGTCAGATGCTG
TTTCGAGGTCGAAGAGCATCCCAGTAATGGTTGTCCTGCCTGCAATATTT
GAATTTTAAATCTAAATCTATTTATTAATATTTAACATTATTTATATGGG
GAATATATTTTTAGACTCATCAATCAAATAAGTATTTATAATAGCAACTT
TTGTGTAATGAAAATGAATATCTATTAATATATGTATTATTTATAATTCC
TATATCCTGTGACTGTCTCACTTAATCCTTTGTTTTCTGACTAATTAGGC
AAGGCTATGTGATTACAAGGCTTTATCTCAGGGGCCAACTAGGCAGCCAA
CCTAAGCAAGATCCCATGGGTTGTGTGTTTATTTCACTTGATGATACAAT
GAACACTTATAAGTGAAGTGATACTATCCAGTTACTGCCGGTTTGAAAAT
ATGCCTGCAATCTGAGCCAGTGCTTTAATGGCATGTCAGACAGAACTTGA
ATGTGTCAGGTGACCCTGATGAAAACATAGCATCTCAGGAGATTTCATGC
CTGGTGCTTCCAAATATTGTTGACAACTGTGACTGTACCCAAATGGAAAG
TAACTCATTTGTTAAAATTATCAATATCTAATATATATGAATAAAGTGTA
AGTTCACAACAAAAAAAAAAAAAAAAAAAAAAAAAAAAAA
>gi|56786138|ref|NP_000610.2| interferon
gamma precursor [Homo sapiens]
(SEQ ID NO: 331)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ
>gi|56786138: 24-166 interferon gamma
mature protein [Homo sapiens]
(SEQ ID NO: 332)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQMLFRGRRASQ

In some embodiments, a control level is a level of IFN-γ in a sample from a control subject (or subjects). Control subjects are described herein. In some embodiments, the control level is a pre-determined threshold. In some embodiments, the control level of IFN-γ is 7.2 pg/mL. In some embodiments, a control level is a level of IFN-γ in a second sample from the same subject from which the first sample was obtained (e.g., a first and second sample may be obtained from the same subject and the comparison between the first and second sample is used to determine if the subject has or is at risk of having Celiac disease). In some embodiments, the first sample and/or second sample is obtained from the subject prior to, during, or after a gluten challenge as described herein.

### Controls and Control Levels

In some embodiments, methods provided herein comprise measuring a level IL-2 in a sample (e.g., a first sample) and then comparing that level to one or more control levels of IL-2.

In some embodiments, a control level is a level of IL-2 in a sample from a control subject (or subjects). In some embodiments, a control subject has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease. In some embodiments, a control subject does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments, a control subject is a healthy individual not having or suspected of having Celiac disease. In some embodiments, the control level is a pre-determined threshold. In some embodiments, a control level is a pre-determined level from a control subject or subjects, such that the control level need not be measured every time the methods described herein are performed.

In some embodiments, a control level is a level of IL-2 in a second sample from the same subject from which the first sample was obtained (e.g., a first and second sample may be obtained from the same subject and the comparison between the first and second sample is used to determine if the subject has or is at risk of having Celiac disease). In some embodiments, the first sample and/or second sample is obtained from the subject prior to, during, or after a gluten challenge as described herein. In some embodiments, the first sample is obtained from the subject after a gluten challenge. In some embodiments, the second sample is obtained from the subject prior to a gluten challenge. In some embodiments, a control level is a level of IL-2 is a negative control level of IL-2. Exemplary negative controls include, but are not limited to, a level of IL-2 in a sample that has been contacted with a non-T cell-activating peptide (e.g., a peptide not recognized by T cells present in a sample from a subject), such as a non-CD4+-T cell-activating peptide, or a T cell response in sample that has not been contacted with a T cell-activating peptide (e.g., contacting the sample with a saline solution or cell culture medium containing no peptides), such as a CD4+ T cell-activating peptide. Additional control samples, e.g., third, fourth, fifth, etc., are also contemplated if additional measurements of IL-2 levels are desired.

### Gluten Peptides and Compositions Containing Gluten Peptides

As used herein the term “gluten peptide” includes any peptide comprising a sequence derived from, or encompassed within, one or more of gluten proteins alpha (α), beta (β), γ (γ) and omega (ω) gliadins, and low and high molecular weight (LMW and HMW) glutenins in wheat, B, C and D hordeins in barley, β, γ and omega secalins in rye, and optionally avenins in oats, including deamidated variants thereof containing one or more glutamine to glutamate substitutions. In some embodiments, the gluten peptide(s) stimulate a CD4+ T cell specific response.

A gluten peptide may include one or more sequences of epitopes known to be recognized by a CD4⁺ T cell in a subject with Celiac disease, e.g., sequences encompassing PELP (SEQ ID NO: 333), PELPY (SEQ ID NO: 334), QPELPYP (SEQ ID NO: 335), PQPELPY (SEQ ID NO: 336), FPQPELP (SEQ ID NO: 337), PELPYPQ (SEQ ID NO: 338), FPQPELPYP (SEQ ID NO: 339), PYPQPELPY (SEQ ID NO: 340), PFPQPELPY (SEQ ID NO: 341), PQPELPYPQ (SEQ ID NO: 342), PFPQPEQPF (SEQ ID NO: 343), PQPEQPFPW (SEQ ID NO: 344), PIPEQPQPY (SEQ ID NO: 345), PQPELPYPQ (SEQ ID NO: 346), FRPEQPYPQ (SEQ ID NO: 347), PQQSFPEQQ (SEQ ID NO: 348), IQPEQPAQL (SEQ ID NO: 349), QQPEQPYPQ (SEQ ID NO: 350), SQPEQEFPQ (SEQ ID NO: 351), PQPEQEFPQ (SEQ ID NO: 352), QQPEQPFPQ (SEQ ID NO: 353), PQPEQPFCQ (SEQ ID NO: 354), QQPFPEQPQ (SEQ ID NO: 355), PFPQPEQPF (SEQ ID NO: 356), PQPEQPFPW (SEQ ID NO: 357), PFSEQEQPV (SEQ ID NO: 358), FSQQQESPF (SEQ ID NO: 359), PFPQPEQPF (SEQ ID NO: 360), PQPEQPFPQ (SEQ ID NO: 361), PIPEQPQPY (SEQ ID NO: 362), PFPQPEQPF (SEQ ID NO: 363), PQPEQPFPQ (SEQ ID NO: 364), PYPEQEEPF (SEQ ID NO: 365), PYPEQEQPF (SEQ ID NO: 366), PFSEQEQPV (SEQ ID NO: 367), EGSFQPSQE (SEQ ID NO: 368), EQPQQPFPQ (SEQ ID NO: 369), EQPQQPYPE (SEQ ID NO: 370), QQGYYPTSPQ (SEQ ID NO: 371), EGSFQPSQE (SEQ ID NO: 372), PQQSFPEQE (SEQ ID NO: 373), or QGYYPTSPQ (SEQ ID NO: 374) (see, e.g., Sollid L M, Qiao S W, Anderson R P, Gianfrani C, Koning F. Nomenclature and listing of celiac disease relevant gluten epitopes recognized by CD4⁺ T cells. Immunogenetics. 2012; 64:455-60; PCT Publication Nos.: WO/2001/025793, WO/2003/104273, WO/2005/105129, and WO/2010/060155). Preferably, in some embodiments, the gluten peptides that comprise sequences of epitopes of less than 6 amino acids also comprise additional amino acids flanking either or both sides of the epitope. Preferably, in some embodiments, the gluten peptides are at least 8 or 9 amino acids in length.

In some embodiments of any one of the methods or kits provided, a gluten peptide may comprise or consist of one or more T cell epitope sequences selected from: PFPQPELPY (SEQ ID NO: 375), PQPELPYPQ (SEQ ID NO: 376), PFPQPEQPF (SEQ ID NO: 377), PQPEQPFPW (SEQ ID NO: 378), EQPIPEQPQ (SEQ ID NO: 379), PIPEQPQPY (SEQ ID NO: 380), PFPQPEQPI (SEQ ID NO: 381), PQPEQPIPV (SEQ ID NO: 382), EQPIPVQPE (SEQ ID NO: 383), PFPQPEQPT (SEQ ID NO: 384), PQPEQPTPI (SEQ ID NO: 385), EQPTPIQPE (SEQ ID NO: 386), PQPEQPFPL (SEQ ID NO: 387), EQPFPLQPE (SEQ ID NO: 388), PQPEQPFSQ (SEQ ID NO: 389), PYPEQPQPF (SEQ ID NO: 390), EGSFQPSQE (SEQ ID NO: 391), QGYYPTSPQ (SEQ ID NO: 392), EQPEQPFPE (SEQ ID NO: 393), EQPFPEQPQ (SEQ ID NO: 394), PFPEQPEQI (SEQ ID NO: 395), PFSEQEQPV (SEQ ID NO: 396), EQPFPEQPI (SEQ ID NO: 397), PFPEQPIPE (SEQ ID NO: 398), PYPQPELPY (SEQ ID NO: 399), PQPELPYPY (SEQ ID NO: 400), and PQPYPEQPQ (SEQ ID NO: 401). In some embodiments of any one of the methods or kits provided, a gluten peptide may comprise or consist of the T cell epitope sequences PFPQPELPY (SEQ ID NO: 402), PQPELPYPQ (SEQ ID NO: 403), PFPQPEQPF (SEQ ID NO: 404), PQPEQPFPW (SEQ ID NO: 405), EQPIPEQPQ (SEQ ID NO: 406), PIPEQPQPY(SEQ ID NO: 407) and at least one further amino acid sequence selected from PFPQPEQPI (SEQ ID NO: 408), PQPEQPIPV (SEQ ID NO: 409), EQPIPVQPE (SEQ ID NO: 410), PFPQPEQPT (SEQ ID NO: 411), PQPEQPTPI (SEQ ID NO: 412), EQPTPIQPE (SEQ ID NO: 413), PQPEQPFPL (SEQ ID NO: 414), EQPFPLQPE (SEQ ID NO: 415), PQPEQPFSQ (SEQ ID NO: 416), PYPEQPQPF (SEQ ID NO: 417), EGSFQPSQE (SEQ ID NO: 418), QGYYPTSPQ (SEQ ID NO: 419), EQPEQPFPE (SEQ ID NO: 420), EQPFPEQPQ (SEQ ID NO: 421), PFPEQPEQI (SEQ ID NO: 422), PFSEQEQPV (SEQ ID NO: 423), EQPFPEQPI (SEQ ID NO: 424), PFPEQPIPE (SEQ ID NO: 425), PYPQPELPY (SEQ ID NO: 426), PQPELPYPY (SEQ ID NO: 427), and PQPYPEQPQ (SEQ ID NO: 428).

In some embodiments of any one of the methods or kits provided, the gluten peptide is selected from:

(a) a first peptide comprising the amino acid sequence PFPQPELPY (SEQ ID NO: 429) and the amino acid sequence PQPELPYPQ (SEQ ID NO: 430);

(b) a second peptide comprising the amino acid sequence PFPQPEQPF (SEQ ID NO: 431) and the amino acid sequence PQPEQPFPW (SEQ ID NO: 432);

(c) a third peptide comprising the amino acid sequence EQPIPEQPQ (SEQ ID NO: 433) and the amino acid sequence PIPEQPQPY (SEQ ID NO: 434);

(d) a fourth peptide comprising the amino acid sequence PFPQPEQPI (SEQ ID NO: 435), the amino acid sequence PQPEQPIPV (SEQ ID NO: 436), and the amino acid sequence EQPIPVQPE (SEQ ID NO: 437);

(e) a fifth peptide comprising the amino acid sequence PFPQPEQPT (SEQ ID NO: 438), the amino acid sequence PQPEQPTPI (SEQ ID NO: 439), and the amino acid sequence EQPTPIQPE (SEQ ID NO: 440);

(f) a sixth peptide comprising the amino acid sequence PFPQPEQPF (SEQ ID NO: 441), the amino acid sequence PQPEQPFPL (SEQ ID NO: 442), and the amino acid sequence EQPFPLQPE (SEQ ID NO: 443);

(g) a seventh peptide comprising the amino acid sequence PFPQPEQPF (SEQ ID NO: 444) and the amino acid sequence PQPEQPFSQ (SEQ ID NO: 445);

(h) an eighth peptide comprising the amino acid sequence PYPEQPQPF (SEQ ID NO: 446);

(i) a ninth peptide comprising the amino acid sequence PFPEQPEQI (SEQ ID NO: 447);

(j) a tenth peptide comprising the amino acid sequence EGSFQPSQE (SEQ ID NO: 448);

(k) an eleventh peptide comprising the amino acid sequence QGYYPTSPQ (SEQ ID NO: 449);

(l) a twelfth peptide comprising the amino acid sequence EQPEQPFPE (SEQ ID NO: 450) and the amino acid sequence EQPFPEQPQ (SEQ ID NO: 451);

(m) a thirteenth peptide comprising the amino acid sequence PFSEQEQPV (SEQ ID NO: 452);

(n) a fourteenth peptide comprising the amino acid sequence EQPFPEQPI (SEQ ID NO: 453), the amino acid sequence PFPEQPIPE (SEQ ID NO: 454), the amino acid sequence EQPIPEQPQ (SEQ ID NO: 455), and the amino acid sequence PIPEQPQPY (SEQ ID NO: 456);

(o) a fifteenth peptide comprising the amino acid sequence PQPELPYPQ (SEQ ID NO: 457) and the amino acid sequence PYPQPELPY (SEQ ID NO: 458);

(p) a sixteenth peptide comprising the amino acid sequence PFPQPELPY (SEQ ID NO: 459) and the amino acid sequence PQPELPYPY (SEQ ID NO: 460);

(q) a seventeenth peptide comprising the amino acid sequence EQPFPEQPI (SEQ ID NO: 461) and the amino acid sequence PFPEQPIPE (SEQ ID NO: 462); and

(r) an eighteenth peptide comprising the amino acid sequence PQPYPEQPQ (SEQ ID NO: 463) and the amino acid sequence PYPEQPQPF (SEQ ID NO: 464). In some embodiments, any one or more of the peptides herein comprises an N-terminal pyroglutamate and/or a C-terminal amide group.

In some embodiments, a gluten peptide may include one or more T cell epitope sequences selected from: PFPQPELPY (SEQ ID NO: 465), PQPELPYPQ (SEQ ID NO: 466), PFPQPEQPF (SEQ ID NO: 467), PQPEQPFPW (SEQ ID NO: 468), PIPEQPQPY (SEQ ID NO: 469), PFPQPEQPIP (SEQ ID NO: 470), EQPIPVQPE (SEQ ID NO: 471), PFPQPEQPTPI (SEQ ID NO: 472), EQPTPIQPE (SEQ ID NO: 473), PQPEQPFPL (SEQ ID NO: 474), EQPFPLQPE (SEQ ID NO: 475), PFPQPEQPF (SEQ ID NO: 476), PQPEQPFSQ (SEQ ID NO: 477), PYPEQPQPF (SEQ ID NO: 478), PFPEQPEQIIP (SEQ ID NO: 479), EGSFQPSQE (SEQ ID NO: 480), QGYYPTSPQ (SEQ ID NO: 481), EQPEQPFPEQPQ (SEQ ID NO: 482), PFSEQEQPV (SEQ ID NO: 483), EQPFPEQPI (SEQ ID NO: 484), PIPEQPQPY (SEQ ID NO: 485), PQPELPYPQ (SEQ ID NO: 486), PYPQPELPY (SEQ ID NO: 487), PFPQPELPY (SEQ ID NO: 488), and PQPELPYPY (SEQ ID NO: 489).

In some embodiments, the gluten peptide is selected from:

(i) a first peptide comprising the amino acid sequence PFPQPELPY (SEQ ID NO: 490) and the amino acid sequence PQPELPYPQ (SEQ ID NO: 491);

(ii) a second peptide comprising the amino acid sequence PFPQPEQPF (SEQ ID NO: 492) and the amino acid sequence PQPEQPFPW (SEQ ID NO: 493);

(iii) a third peptide comprising the amino acid sequence PIPEQPQPY (SEQ ID NO: 494);

(iv) a fourth peptide comprising the amino acid sequence PFPQPEQPIP (SEQ ID NO: 495) and the amino acid sequence EQPIPVQPE (SEQ ID NO: 496);

(v) a fifth peptide comprising the amino acid sequence PFPQPEQPTPI (SEQ ID NO: 497) and the amino acid sequence EQPTPIQPE (SEQ ID NO: 498);

(vi) a sixth peptide comprising the amino acid sequence PQPEQPFPL (SEQ ID NO: 499) and the amino acid sequence EQPFPLQPE (SEQ ID NO: 500);

(vii) a seventh peptide comprising the amino acid sequence PFPQPEQPF (SEQ ID NO: 501) and the amino acid sequence PQPEQPFSQ (SEQ ID NO: 502);

(viii) an eighth peptide comprising the amino acid sequence PYPEQPQPF (SEQ ID NO: 503);

(ix) a ninth peptide comprising the amino acid sequence PFPEQPEQIIP (SEQ ID NO: 504);

(x) a tenth peptide comprising the amino acid sequence EGSFQPSQE (SEQ ID NO: 505);

(xi) an eleventh peptide comprising the amino acid sequence QGYYPTSPQ (SEQ ID NO: 506);

(xii) a twelfth peptide comprising the amino acid sequence EQPEQPFPEQPQ (SEQ ID NO: 507);

(xiii) a thirteenth peptide comprising the amino acid sequence PFSEQEQPV (SEQ ID NO: 508);

(xiv) a fourteenth peptide comprising the amino acid sequence EQPFPEQPI (SEQ ID NO: 509) and PIPEQPQPY (SEQ ID NO: 510);

(xv) a fifteenth peptide comprising the amino acid sequence PQPELPYPQ (SEQ ID NO: 511) and the amino acid sequence PYPQPELPY (SEQ ID NO: 512);

(xvi) a sixteenth peptide comprising the amino acid sequence PFPQPELPY (SEQ ID NO: 513) and the amino acid sequence PQPELPYPY (SEQ ID NO: 514); and

(xvii) a seventeenth peptide comprising the amino acid sequence EQPFPEQPI (SEQ ID NO: 515). In some embodiments, any one of the peptides herein comprises an N-terminal pyroglutamate and/or a C-terminal amide group.

Exemplary gluten peptides and method for synthesizing or obtaining such peptides are known in the art and described herein (see, e.g., PCT Publication Nos.: WO/2001/025793, WO/2003/104273, WO/2005/105129, and WO/2010/060155, which are incorporated herein by reference in their entirety, including specifically the aforementioned peptides and methods). A gluten peptide can be recombinantly and/or synthetically produced. In some embodiments, a gluten peptide is chemically synthesized, e.g., using a method known in the art. Non-limiting examples of peptide synthesis include liquid-phase synthesis and solid-phase synthesis. In some embodiments, a gluten peptide is produced by enzymatic digestion, e.g., by enzymatic digestion of a larger polypeptide into short peptides.

In some embodiments, one or more glutamate residues of a gluten peptide may be generated by tissue transglutaminase (tTG) deamidation activity upon one or more glutamine residues of the gluten peptide. This deamidation of glutamine to glutamate can cause the generation of gluten peptides that can bind to HLA-DQ2 or -DQ8 molecules with high affinity. This reaction may occur in vitro by contacting the gluten peptide composition with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 516) could become PFPQPELPY (SEQ ID NO: 517) after processing by tTG. Conservative substitution of E with D is also contemplated herein in any one of the peptides provided herein (e.g., PFPQPELPY (SEQ ID NO: 518) could become PFPQPDLPY (SEQ ID NO: 519)). Exemplary peptides including an E to D substitution include peptides comprising or consisting of one or more of the sequences selected from PFPQPDLPY (SEQ ID NO: 520), PQPDLPYPQ (SEQ ID NO: 521), PFPQPDQPF (SEQ ID NO: 522), PQPDQPFPW (SEQ ID NO: 523), PIPDQPQPY (SEQ ID NO: 524), LQPFPQPDLPYPQPQ (SEQ ID NO: 525), QPFPQPDQPFPWQP (SEQ ID NO: 526), PQQPIPDQPQPYPQQ (SEQ ID NO: 527), PFPQPDQPIP (SEQ ID NO: 528), DQPIPVQPD (SEQ ID NO: 529), PFPQPDQPTPI (SEQ ID NO: 530), DQPTPIQPD (SEQ ID NO: 531), PQPDQPFPL (SEQ ID NO: 532), DQPFPLQPD (SEQ ID NO: 533), PFPQPDQPF (SEQ ID NO: 534), PQPDQPFSQ (SEQ ID NO: 535), PYPDQPQPF (SEQ ID NO: 536), PFPDQPDQIIP (SEQ ID NO: 537), DGSFQPSQD (SEQ ID NO: 538), DQPDQPFPDQPQ (SEQ ID NO: 539), PFSDQDQPV (SEQ ID NO: 540), DQPFPDQPI (SEQ ID NO: 541), PIPDQPQPY (SEQ ID NO: 542), PQPDLPYPQ (SEQ ID NO: 543), PYPQPDLPY (SEQ ID NO: 544), PFPQPDLPY (SEQ ID NO: 545), and PQPDLPYPY (SEQ ID NO: 546). Such substituted peptides can be the gluten peptides of any one of the methods and compositions provided herein.

In some embodiments, it may be desirable to utilize the non-deamidated forms of such peptides, e.g., if the peptides are contained within a composition for administration to a subject where tissue transglutaminase will act in situ (see, e.g., Øyvind Molberg, Stephen McAdam, Knut E. A. Lundin, Christel Kristiansen, Helene Arentz-Hansen, Kjell Kett and Ludvig M. Sollid. T cells from celiac disease lesions recognize gliadin epitopes deamidated in situ by endogenous tissue transglutaminase. Eur. J. Immunol. 2001. 31: 1317-1323). Accordingly, gluten peptides that have not undergone deamidation are also contemplated herein (e.g., gluten peptides comprising or consisting of one or more of the sequences selected from: PQLP (SEQ ID NO: 547), PQLPY (SEQ ID NO: 548), QPQLPYP (SEQ ID NO: 549), PQPQLPY (SEQ ID NO: 550), FPQPQLP (SEQ ID NO: 551), PQLPYPQ (SEQ ID NO: 552), FPQPQLPYP (SEQ ID NO: 553), PYPQPQLPY (SEQ ID NO: 554), PFPQPQLPY (SEQ ID NO: 555), PQPQLPYPQ (SEQ ID NO: 556), PFPQPQQPF (SEQ ID NO: 557), PQPQQPFPW (SEQ ID NO: 558), PIPQQPQPY (SEQ ID NO: 559), LQPFPQPQLPYPQPQ (SEQ ID NO: 560), QPFPQPQQPFPWQP (SEQ ID NO: 561), PEQPIPQQPQPYPQQ (SEQ ID NO: 562), PQPQLPYPQ (SEQ ID NO: 563), FRPQQPYPQ (SEQ ID NO: 564), PQQSFPQQQ (SEQ ID NO: 565), IQPQQPAQL (SEQ ID NO: 566), QQPQQPYPQ (SEQ ID NO: 567), SQPQQQFPQ (SEQ ID NO: 568), PQPQQQFPQ (SEQ ID NO: 569), QQPQQPFPQ (SEQ ID NO: 570), PQPQQPFCQ (SEQ ID NO: 571), QQPFPQQPQ (SEQ ID NO: 572), PFPQPQQPF (SEQ ID NO: 573), PQPQQPFPW (SEQ ID NO: 574), PFSQQQQPV (SEQ ID NO: 575), FSQQQQSPF (SEQ ID NO: 576), PFPQPQQPF (SEQ ID NO: 577), PQPQQPFPQ (SEQ ID NO: 578), PIPQQPQPY (SEQ ID NO: 579), PFPQPQQPF (SEQ ID NO: 580), PQPQQPFPQ (SEQ ID NO: 581), PYPEQQEPF (SEQ ID NO: 582), PYPEQQQPF (SEQ ID NO: 583), PFSQQQQPV (SEQ ID NO: 584), QGSFQPSQQ (SEQ ID NO: 585), QQPQQPFPQ (SEQ ID NO: 586), QQPQQPYPQ (SEQ ID NO: 587), QQGYYPTSPQ (SEQ ID NO: 588), QGSFQPSQQ (SEQ ID NO: 589), PQQSFPQQQ (SEQ ID NO: 590), QGYYPTSPQ (SEQ ID NO: 591), LQPFPQPELPYPQPQ (SEQ ID NO: 592), QPFPQPQQPFPWQP (SEQ ID NO: 593), PQQPIPQQPQPYPQQ (SEQ ID NO: 594), PFPQPEQPIP (SEQ ID NO: 595), EQPIPVQPE (SEQ ID NO: 596), PFPQPEQPTPI (SEQ ID NO: 597), EQPTPIQPE (SEQ ID NO: 598), PQPEQPFPL (SEQ ID NO: 599), EQPFPLQPE (SEQ ID NO: 600), PFPQPEQPF (SEQ ID NO: 601), PQPEQPFSQ (SEQ ID NO: 602), PYPEQPQPF (SEQ ID NO: 603), PFPEQPEQIIP (SEQ ID NO: 604), EGSFQPSQE (SEQ ID NO: 605), EQPEQPFPEQPQ (SEQ ID NO: 606), PFSEQEQPV (SEQ ID NO: 607), EQPFPEQPI (SEQ ID NO: 608), PIPEQPQPY (SEQ ID NO: 609), PQPELPYPQ (SEQ ID NO: 610), PYPQPELPY (SEQ ID NO: 611), PFPQPELPY (SEQ ID NO: 612), PQPELPYPY (SEQ ID NO: 613), LQPFPQPQLPYPQPQ (SEQ ID NO: 614), QPFPQPQQPFPWQP (SEQ ID NO: 615), PQQPIPQQPQPYPQQ (SEQ ID NO: 616), QPFPQPQQPIPVQPQQS (SEQ ID NO: 617), QPFPQPQQPTPIQPQQP (SEQ ID NO: 618), QPFPQPQQPFPLQPQQP (SEQ ID NO: 619), QPFPQPQQPFSQQ (SEQ ID NO: 620), PQPYPQQPQPFPQQ (SEQ ID NO: 621), QPFPEQPQQIIPQQP (SEQ ID NO: 622), SGEGSFQPSQQNPQ (SEQ ID NO: 623), PQQPQQPFPQQPQQ (SEQ ID NO: 624), QPPFSQQQQPVLPQ (SEQ ID NO: 625), PQQPFPQQPIPQQPQPYP (SEQ ID NO: 626), QPYPQPQLPYPQPQ (SEQ ID NO: 627), and QPFPQPQLPYPYPQ (SEQ ID NO: 628).

A gluten peptide may also be an analog of any one of the peptides described herein. Preferably, in some embodiments the analog is recognized by a CD4⁺ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any one of the peptides provided, such variants can include conservative amino acid substitutions, e.g., E to D substitution.

In some embodiments, analogs may include one or more amino acid substitutions as shown in Table A (see, e.g., Anderson et al. Antagonists and non-toxic variants of the dominant wheat gliadin T cell epitope in coeliac disease. Gut. 2006 April; 55(4): 485-491; and PCT Publication WO2003104273, the contents of which are incorporated herein by reference, including the aforementioned analogs). The gluten peptides provided herein include analogs of FPQPELPYP (SEQ ID NO: 629) comprising one or more of the listed amino acid substitutions. In some embodiments, the analog is an analog of FPQPELPYP (SEQ ID NO: 629) comprising one of the amino acid substitutions provided in Table A below.

TABLE A
Exemplary substitutions in the core
sequence FPQPELPYP (SEQ ID NO: 629)
encompassed within the 17mer
QLQPFPQPELPYPQPQS (SEQ ID NO: 630)

| Amino | | | | | | | | | |
|---|---|---|---|---|---|---|---|---|---|
| acid in | | | | | | | | | |
| epitope | F | P | Q | P | E | L | P | Y | P |
| Exem- | A, G, | A, F, | A, F, | — | D | M | S | I, | S, |
| plary | H, I, | I, M, | G, H, | | | | | S, | T, |
| Substi- | L, M | S, T, | I, L, | | | | | V, | Y |
| tutions | P, S, | V, W, | M, S, | | | | | W | |
| | T, W, | Y | T, V | | | | | | |
| | Y | | | | | | | | |

The length of the peptides may vary. In some embodiments, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments, peptides are, e.g., 4-100, 4-50, 4-40, 4-30, or 4-20 amino acids in length. In some embodiments, peptides are 4-20, 5-20, 6-20, 7-20, 8-20, 9-20, 10-20, 11-20, 12-20, 13-20, 14-20, or 15-20 amino acids in length. In some embodiments, peptides are e.g., 5-30, 10-30, 15-30 or 20-30 amino acids in length. In some embodiments, peptides are 4-50, 5-50, 6-50, 7-50, 8-50, 9-50, 10-50, 11-50, 12-50, 13-50, 14-50, or 15-50 amino acids in length. In some embodiments, peptides are 8-30 amino acids in length.

In some embodiments of any one of the methods provided herein, a composition comprising one or one or more gluten peptide(s) is contemplated. In some embodiments, the composition comprises at least one (e.g., 1, 2, 3, 4, 5, 7, 8, 9, 10, 11, 12, 13, 14, 15, or more) peptide, the at least one peptide comprising at least one (e.g., 1, 2, 3, 4, 5, 7, 8, 9, 10, 11, 12, 13, 14, 15, or more) amino acid sequence(s) selected from PFPQPELPY (SEQ ID NO: 631), PQPELPYPQ (SEQ ID NO: 632), PFPQPEQPF (SEQ ID NO: 633), PQPEQPFPW (SEQ ID NO: 634), EQPIPEQPQ (SEQ ID NO: 635), PIPEQPQPY (SEQ ID NO: 636), PFPQPEQPI (SEQ ID NO: 637), PQPEQPIPV (SEQ ID NO: 638), EQPIPVQPE (SEQ ID NO: 639), PFPQPEQPT (SEQ ID NO: 640), PQPEQPTPI (SEQ ID NO: 641), EQPTPIQPE (SEQ ID NO: 642), PQPEQPFPL (SEQ ID NO: 643), EQPFPLQPE (SEQ ID NO: 644), PQPEQPFSQ (SEQ ID NO: 645), PYPEQPQPF (SEQ ID NO: 646), EGSFQPSQE (SEQ ID NO: 647), QGYYPTSPQ (SEQ ID NO: 648), EQPEQPFPE (SEQ ID NO: 649), EQPFPEQPQ (SEQ ID NO: 650), PFPEQPEQI (SEQ ID NO: 651), PFSEQEQPV (SEQ ID NO: 652), EQPFPEQPI (SEQ ID NO: 653), PFPEQPIPE (SEQ ID NO: 654), PYPQPELPY (SEQ ID NO: 655), PQPELPYPY (SEQ ID NO: 656), and PQPYPEQPQ (SEQ ID NO: 657).

In some embodiments, the composition comprises at least one (e.g., 1, 2, 3, 4, 5, 7, 8, 9, 10, 11, 12, 13, 14, 15, or more) peptide, the at least one peptide comprising at least one (e.g., 1, 2, 3, 4, 5, 7, 8, 9, 10, 11, 12, 13, 14, 15, or more) amino acid sequence(s) selected from PFPQPELPY (SEQ ID NO: 658), PQPELPYPQ (SEQ ID NO: 659), PFPQPEQPF (SEQ ID NO: 660), PQPEQPFPW (SEQ ID NO: 661), PIPEQPQPY (SEQ ID NO: 662), PFPQPEQPIP (SEQ ID NO: 663), EQPIPVQPE (SEQ ID NO: 664), PFPQPEQPTPI (SEQ ID NO: 665), EQPTPIQPE (SEQ ID NO: 666), PQPEQPFPL (SEQ ID NO: 667), EQPFPLQPE (SEQ ID NO: 668), PFPQPEQPF (SEQ ID NO: 669), PQPEQPFSQ (SEQ ID NO: 670), PYPEQPQPF (SEQ ID NO: 671), PFPEQPEQIIP (SEQ ID NO: 672), EGSFQPSQE (SEQ ID NO: 673), QGYYPTSPQ (SEQ ID NO: 674), EQPEQPFPEQPQ (SEQ ID NO: 675), PFSEQEQPV (SEQ ID NO: 676), EQPFPEQPI (SEQ ID NO: 677), PIPEQPQPY (SEQ ID NO: 678), PQPELPYPQ (SEQ ID NO: 679), PYPQPELPY (SEQ ID NO: 680), PFPQPELPY (SEQ ID NO: 681), PQPELPYPY (SEQ ID NO: 682), and EQPFPEQPI (SEQ ID NO: 683).

In some embodiments of any one of the methods provided herein, the composition comprises at least one of:

(a) a first peptide comprising the amino acid sequence PFPQPELPY (SEQ ID NO: 684) and the amino acid sequence PQPELPYPQ (SEQ ID NO: 685);

(b) a second peptide comprising the amino acid sequence PFPQPEQPF (SEQ ID NO: 686) and the amino acid sequence PQPEQPFPW (SEQ ID NO: 687);

(c) a third peptide comprising the amino acid sequence EQPIPEQPQ (SEQ ID NO: 688) and the amino acid sequence PIPEQPQPY (SEQ ID NO: 689);

(d) a fourth peptide comprising the amino acid sequence PFPQPEQPI (SEQ ID NO: 690), the amino acid sequence PQPEQPIPV (SEQ ID NO: 691), and the amino acid sequence EQPIPVQPE (SEQ ID NO: 692);

(e) a fifth peptide comprising the amino acid sequence PFPQPEQPT (SEQ ID NO: 693), the amino acid sequence PQPEQPTPI (SEQ ID NO: 694), and the amino acid sequence EQPTPIQPE (SEQ ID NO: 695);

(f) a sixth peptide comprising the amino acid sequence PFPQPEQPF (SEQ ID NO: 696), the amino acid sequence PQPEQPFPL (SEQ ID NO: 697), and the amino acid sequence EQPFPLQPE (SEQ ID NO: 698);

(g) a seventh peptide comprising the amino acid sequence PFPQPEQPF (SEQ ID NO: 699) and the amino acid sequence PQPEQPFSQ (SEQ ID NO: 700);

(h) an eighth peptide comprising the amino acid sequence PYPEQPQPF (SEQ ID NO: 701);

(i) a ninth peptide comprising the amino acid sequence PFPEQPEQI (SEQ ID NO: 702);

(j) a tenth peptide comprising the amino acid sequence EGSFQPSQE (SEQ ID NO: 703);

(k) an eleventh peptide comprising the amino acid sequence QGYYPTSPQ (SEQ ID NO: 704);

(l) a twelfth peptide comprising the amino acid sequence EQPEQPFPE (SEQ ID NO: 705) and the amino acid sequence EQPFPEQPQ (SEQ ID NO: 706);

(m) a thirteenth peptide comprising the amino acid sequence PFSEQEQPV (SEQ ID NO: 707);

(n) a fourteenth peptide comprising the amino acid sequence EQPFPEQPI (SEQ ID NO: 708), the amino acid sequence PFPEQPIPE (SEQ ID NO: 709), the amino acid sequence EQPIPEQPQ (SEQ ID NO: 710), and the amino acid sequence PIPEQPQPY (SEQ ID NO: 711);

(o) a fifteenth peptide comprising the amino acid sequence PQPELPYPQ (SEQ ID NO: 712) and the amino acid sequence PYPQPELPY (SEQ ID NO: 713);

(p) a sixteenth peptide comprising the amino acid sequence PFPQPELPY (SEQ ID NO: 714) and the amino acid sequence PQPELPYPY (SEQ ID NO: 715);

(q) a seventeenth peptide comprising the amino acid sequence EQPFPEQPI (SEQ ID NO: 716) and the amino acid sequence PFPEQPIPE (SEQ ID NO: 717); and

(r) an eighteenth peptide comprising the amino acid sequence PQPYPEQPQ (SEQ ID NO: 718) and the amino acid sequence PYPEQPQPF (SEQ ID NO: 719).

In some embodiments,

(a) the first peptide comprises the amino acid sequence LQPFPQPELPYPQPQ (SEQ ID NO: 720);

(b) the second peptide comprises the amino acid sequence QPFPQPEQPFPWQP (SEQ ID NO: 721);

(c) the third peptide comprises the amino acid sequence PEQPIPEQPQPYPQQ (SEQ ID NO: 722);

(d) the fourth peptide comprises the amino acid sequence QPFPQPEQPIPVQPEQS (SEQ ID NO: 723);

(e) the fifth peptide comprises the amino acid sequence QPFPQPEQPTPIQPEQP (SEQ ID NO: 724);

(f) the sixth peptide comprises the amino acid sequence QPFPQPEQPFPLQPEQP (SEQ ID NO: 725);

(g) the seventh peptide comprises the amino acid sequence QPFPQPEQPFSQQ (SEQ ID NO: 726);

(h) the eighth peptide comprises the amino acid sequence PQPYPEQPQPFPQQ (SEQ ID NO: 727);

(i) the ninth peptide comprises the amino acid sequence QPFPEQPEQIIPQQP (SEQ ID NO: 728);

(j) the tenth peptide comprises the amino acid sequence SGEGSFQPSQENPQ (SEQ ID NO: 729);

(k) the eleventh peptide comprises the amino acid sequence GQQGYYPTSPQQSG (SEQ ID NO: 730);

(l) the twelfth peptide comprises the amino acid sequence PEQPEQPFPEQPQQ (SEQ ID NO: 731);

(m) the thirteenth peptide comprises the amino acid sequence QPPFSEQEQPVLPQ (SEQ ID NO: 732);

(n) the fourteenth peptide comprises the amino acid sequence PEQPFPEQPIPEQPQPYP (SEQ ID NO: 733);

(o) the fifteenth peptide comprises the amino acid sequence QPYPQPELPYPQPQ (SEQ ID NO: 734);

(p) the sixteenth peptide comprises the amino acid sequence QPFPQPELPYPYPQ (SEQ ID NO: 735);

(q) the seventeenth peptide comprises the amino acid sequence PQEQPFPEQPIPEQP (SEQ ID NO: 736); and

(r) the eighteenth peptide comprises the amino acid sequence QPQPYPEQPQPFPQQ (SEQ ID NO: 737).

In some embodiments of any one of the methods provided herein, the composition comprises at least one peptide, the at least one peptide comprising at least one amino acid sequence selected from PFPQPELPY (SEQ ID NO: 738), PQPELPYPQ (SEQ ID NO: 739), PFPQPEQPF (SEQ ID NO: 740), PQPEQPFPW (SEQ ID NO: 741), EQPIPEQPQ (SEQ ID NO: 742), PIPEQPQPY (SEQ ID NO: 743), PFPQPEQPI (SEQ ID NO: 744), PQPEQPIPV (SEQ ID NO: 745), EQPIPVQPE (SEQ ID NO: 746), PFPQPEQPT (SEQ ID NO: 747), PQPEQPTPI (SEQ ID NO: 748), EQPTPIQPE (SEQ ID NO: 749), PQPEQPFPL (SEQ ID NO: 750), EQPFPLQPE (SEQ ID NO: 751), EGSFQPSQE (SEQ ID NO: 752), QGYYPTSPQ (SEQ ID NO: 753), EQPEQPFPE (SEQ ID NO: 754), PFSEQEQPV (SEQ ID NO: 755), PYPQPELPY (SEQ ID NO: 756), EQPFPEQPI (SEQ ID NO: 757), PFPEQPIPE (SEQ ID NO: 758), PYPEQPQPF (SEQ ID NO: 759), and PQPYPEQPQ (SEQ ID NO: 760). In some embodiments of any one of the methods provided herein, the composition comprises at least one (e.g., two, three, four, five, six, seven, eight, nine, ten, eleven, twelve, thirteen, fourteen, fifteen, or sixteen) peptide comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 761), PQPELPYPQ (SEQ ID NO: 762), PFPQPEQPF (SEQ ID NO: 763), PQPEQPFPW (SEQ ID NO: 764), EQPIPEQPQ (SEQ ID NO: 765), PIPEQPQPY (SEQ ID NO: 766), PFPQPEQPI (SEQ ID NO: 767), PQPEQPIPV (SEQ ID NO: 768), EQPIPVQPE (SEQ ID NO: 769), PFPQPEQPT (SEQ ID NO: 770), PQPEQPTPI (SEQ ID NO: 771), EQPTPIQPE (SEQ ID NO: 772), PQPEQPFPL (SEQ ID NO: 773), EQPFPLQPE (SEQ ID NO: 774), EGSFQPSQE (SEQ ID NO: 775), QGYYPTSPQ (SEQ ID NO: 776), EQPEQPFPE (SEQ ID NO: 777), PFSEQEQPV (SEQ ID NO: 778), PYPQPELPY (SEQ ID NO: 779), EQPFPEQPI (SEQ ID NO: 780), PFPEQPIPE (SEQ ID NO: 781), PYPEQPQPF (SEQ ID NO: 782), and PQPYPEQPQ (SEQ ID NO: 783).

In some embodiments of any one of the methods provided herein, the composition comprises at least one of:

(a) a first peptide comprising the amino acid sequence PFPQPELPY (SEQ ID NO: 784) and the amino acid sequence PQPELPYPQ (SEQ ID NO: 785);

(b) a second peptide comprising the amino acid sequence PFPQPEQPF (SEQ ID NO: 786) and the amino acid sequence PQPEQPFPW (SEQ ID NO: 787);

(c) a third peptide comprising the amino acid sequence EQPIPEQPQ (SEQ ID NO: 788) and the amino acid sequence PIPEQPQPY (SEQ ID NO: 789);

(d) a fourth peptide comprising the amino acid sequence PFPQPEQPI (SEQ ID NO: 790) and the amino acid sequence PQPEQPIPV (SEQ ID NO: 791);

(e) a fifth peptide comprising the amino acid sequence EQPIPVQPE (SEQ ID NO: 792);

(f) a sixth peptide comprising the amino acid sequence PFPQPEQPT (SEQ ID NO: 793) and the amino acid sequence PQPEQPTPI (SEQ ID NO: 794);

(g) a seventh peptide comprising the amino acid sequence EQPTPIQPE (SEQ ID NO: 795);

(h) an eighth peptide comprising the amino acid sequence PQPEQPFPL (SEQ ID NO: 796);

(i) a ninth peptide comprising the amino acid sequence EQPFPLQPE (SEQ ID NO: 797);

(j) a tenth peptide comprising the amino acid sequence EGSFQPSQE (SEQ ID NO: 798);

(k) a eleventh peptide comprising the amino acid sequence QGYYPTSPQ (SEQ ID NO: 799);

(l) a twelfth peptide comprising the amino acid sequence EQPEQPFPE (SEQ ID NO: 800);

(m) a thirteenth peptide comprising the amino acid sequence PFSEQEQPV (SEQ ID NO: 801);

(n) a fourteenth peptide comprising the amino acid sequence PYPQPELPY (SEQ ID NO: 802);

(o) a fifteenth peptide comprising the amino acid sequence EQPFPEQPI (SEQ ID NO: 803) and the amino acid sequence PFPEQPIPE (SEQ ID NO: 804); and

(p) a sixteenth peptide comprising the amino acid sequence PYPEQPQPF (SEQ ID NO: 805) and the amino acid sequence PQPYPEQPQ (SEQ ID NO: 806).

In some embodiments:

(a) the first peptide comprises the amino acid sequence PFPQPELPYPQP (SEQ ID NO: 807);

(b) the second peptide comprises the amino acid sequence PFPQPEQPFPWQ (SEQ ID NO: 808);

(c) the third peptide comprises the amino acid sequence EQPIPEQPQPYP (SEQ ID NO: 809);

(d) the fourth peptide comprises the amino acid sequence PFPQPEQPIPVQ (SEQ ID NO: 810);

(e) the fifth peptide comprises the amino acid sequence PEQPIPVQPEQS (SEQ ID NO: 811);

(f) the sixth peptide comprises the amino acid sequence PFPQPEQPTPIQ (SEQ ID NO: 812);

(g) the seventh peptide comprises the amino acid sequence PEQPTPIQPEQP (SEQ ID NO: 813);

(h) the eighth peptide comprises the amino acid sequence PFPQPEQPFPLQ (SEQ ID NO: 814);

(i) the ninth peptide comprises the amino acid sequence PEQPFPLQPEQP (SEQ ID NO: 815);

(j) the tenth peptide comprises the amino acid sequence GEGSFQPSQENP (SEQ ID NO: 816);

(k) the eleventh peptide comprises the amino acid sequence QQGYYPTSPQQS (SEQ ID NO: 817);

(l) the twelfth peptide comprises the amino acid sequence PEQPEQPFPEQP (SEQ ID NO: 818);

(m) the thirteenth peptide comprises the amino acid sequence PPFSEQEQPVLP (SEQ ID NO: 819);

(n) the fourteenth peptide comprises the amino acid sequence PYPQPELPYPQP (SEQ ID NO: 820);

(o) the fifteenth peptide comprises the amino acid sequence EQPFPEQPIPEQ (SEQ ID NO: 821); and

(p) the sixteenth peptide comprises the amino acid sequence PQPYPEQPQPFP (SEQ ID NO: 822).

In some embodiments of any one of the methods provided herein, the composition comprises at least four (e.g., five, six, seven, eight, nine, ten, eleven, twelve, thirteen, fourteen, fifteen or sixteen) of the peptides. In some embodiments of any one of the methods provided herein, the composition comprises (or consists of) the peptides in (a)-(p). In some embodiments of any one of the methods provided herein, at least one of the peptides comprises an N-terminal pyroglutamate and/or a C-terminal amide group. In some embodiments of any one of the methods provided herein, each of the peptides comprises an N-terminal pyroglutamate and/or a C-terminal amide group.

In some embodiments of any one of the methods provided herein, the composition comprises at least one of:

(i) a first peptide comprising the amino acid sequence PFPQPELPY (SEQ ID NO: 823) and the amino acid sequence PQPELPYPQ (SEQ ID NO: 824);
(ii) a second peptide comprising the amino acid sequence PFPQPEQPF (SEQ ID NO: 825) and the amino acid sequence PQPEQPFPW (SEQ ID NO: 826);
(iii) a third peptide comprising the amino acid sequence PIPEQPQPY (SEQ ID NO: 827);
(iv) a fourth peptide comprising the amino acid sequence PFPQPEQPIP (SEQ ID NO: 828) and the amino acid sequence EQPIPVQPE (SEQ ID NO: 829);
(v) a fifth peptide comprising the amino acid sequence PFPQPEQPTPI (SEQ ID NO: 830) and the amino acid sequence EQPTPIQPE (SEQ ID NO: 831);
(vi) a sixth peptide comprising the amino acid sequence PQPEQPFPL (SEQ ID NO: 832) and the amino acid sequence EQPFPLQPE (SEQ ID NO: 833);
(vii) a seventh peptide comprising the amino acid sequence PFPQPEQPF (SEQ ID NO: 834) and the amino acid sequence PQPEQPFSQ (SEQ ID NO: 835);
(viii) an eighth peptide comprising the amino acid sequence PYPEQPQPF (SEQ ID NO: 836);
(ix) a ninth peptide comprising the amino acid sequence PFPEQPEQIIP (SEQ ID NO: 837);
(x) a tenth peptide comprising the amino acid sequence EGSFQPSQE (SEQ ID NO: 838);
(xi) an eleventh peptide comprising the amino acid sequence QGYYPTSPQ (SEQ ID NO: 839);
(xii) a twelfth peptide comprising the amino acid sequence EQPEQPFPEQPQ (SEQ ID NO: 840);
(xiii) a thirteenth peptide comprising the amino acid sequence PFSEQEQPV (SEQ ID NO: 841);
(xiv) a fourteenth peptide comprising the amino acid sequence EQPFPEQPI (SEQ ID NO: 842) and PIPEQPQPY (SEQ ID NO: 843);
(xv) a fifteenth peptide comprising the amino acid sequence PQPELPYPQ (SEQ ID NO: 844) and the amino acid sequence PYPQPELPY (SEQ ID NO: 845);
(xvi) a sixteenth peptide comprising the amino acid sequence PFPQPELPY (SEQ ID NO: 846) and the amino acid sequence PQPELPYPY (SEQ ID NO: 847); and
(xvii) a seventeenth peptide comprising the amino acid sequence EQPFPEQPI (SEQ ID NO: 848).

In some embodiments of any one of the methods provided herein,

(i) the first peptide comprises the amino acid sequence LQPFPQPELPYPQPQ (SEQ ID NO: 849);

(ii) the second peptide comprises the amino acid sequence QPFPQPEQPFPWQP (SEQ ID NO: 850);

(iii) the third peptide comprises the amino acid sequence PEQPIPEQPQPYPQQ (SEQ ID NO: 851);

(iv) the fourth peptide comprises the amino acid sequence QPFPQPEQPIPVQPEQS (SEQ ID NO: 852);

(v) the fifth peptide comprises the amino acid sequence QPFPQPEQPTPIQPEQP (SEQ ID NO: 853);

(vi) the sixth peptide comprises the amino acid sequence QPFPQPEQPFPLQPEQP (SEQ ID NO: 854);

(vii) the seventh peptide comprises the amino acid sequence QPFPQPEQPFSQQ (SEQ ID NO: 855);

(viii) the eighth peptide comprises the amino acid sequence PQPYPEQPQPFPQQ (SEQ ID NO: 856);

(ix) the ninth peptide comprises the amino acid sequence QPFPEQPEQIIPQQP (SEQ ID NO: 857);

(x) the tenth peptide comprises the amino acid sequence SGEGSFQPSQENPQ (SEQ ID NO: 858);

(xi) the eleventh peptide comprises the amino acid sequence GQQGYYPTSPQQSG (SEQ ID NO: 859);

(xii) the twelfth peptide comprises the amino acid sequence PEQPEQPFPEQPQQ (SEQ ID NO: 860);

(xiii) the thirteenth peptide comprises the amino acid sequence QPPFSEQEQPVLPQ (SEQ ID NO: 861);

(xiv) the fourteenth peptide comprises the amino acid sequence PEQPFPEQPIPEQPQPYP (SEQ ID NO: 862);

(xv) the fifteenth peptide comprises the amino acid sequence QPYPQPELPYPQPQ (SEQ ID NO: 863);

(xvi) the sixteenth peptide comprises the amino acid sequence QPFPQPELPYPYPQ (SEQ ID NO: 864); and

(xvii) the seventeenth peptide comprises the amino acid sequence EQPFPEQPI (SEQ ID NO: 865).

“First”, “second”, “third”, etc. are not meant to imply an order of use or importance, unless specifically stated otherwise. In some embodiments, the peptides are each individually 8-50 amino acids in length. In some embodiments, the composition comprises at least one, at least two, at least three, at least four, at least five, at least six, at least seven, at least eight, at least nine, at least ten, at least eleven, at least twelve, at least thirteen or at least fourteen of the peptides. In some embodiments, the composition comprises the first, second, and third peptides. In some embodiments, the composition comprises the first, second, third, fourth, fifth, sixth, seventh, eighth, ninth, tenth, eleventh, twelfth, and thirteenth peptides. In some embodiments, the composition comprises the second, fourth, fifth, sixth, seventh, eighth, ninth, tenth, eleventh, twelfth, thirteenth, fourteenth, fifteenth, and sixteenth peptides. In some embodiments, the composition comprises the first, second, third, fourth, fifth, sixth, eighth, ninth, tenth, eleventh, twelfth, and thirteenth peptides. In some embodiments, the composition comprises the second, fourth, fifth, sixth, eighth, ninth, tenth, eleventh, twelfth, thirteenth, fourteenth, fifteenth, and sixteenth peptides. In some embodiments, the composition comprises the second, third, fourth, fifth, sixth, seventh, eighth, ninth, tenth, eleventh, twelfth, thirteenth, fifteenth, sixteenth, and seventeenth peptides. In some embodiments, the composition comprises the second, third, fourth, fifth, sixth, eighth, ninth, tenth, eleventh, twelfth, thirteenth, fifteenth, sixteenth, and seventeenth peptides. In some embodiments, at least one of the peptides comprises an N-terminal pyroglutamate and/or a C-terminal amide group. In some embodiments, each of the peptides comprises an N-terminal pyroglutamate and/or a C-terminal amide group. Any one of the aforementioned compositions or peptide combinations may be used in any one of the methods provided herein. In some embodiments, each of the peptides are present in an amount of 2.5 ug/mL in the composition. In some embodiments, each of the peptides are present in an amount of 5 ug/mL in the composition. In some embodiments, each of the peptides are present in an amount of 10 ug/mL in the composition. In some embodiments, each of the peptides are present in an amount of 20 ug/mL in the composition. In some embodiments, each of the peptides are present in an amount of 25 ug/mL in the composition. In some embodiments, each of the peptides are present in an amount of 50 ug/mL in the composition. In some embodiments, each of the peptides are present in an amount of 5 uM in the composition. In some embodiments, each of the peptides are present in an amount of 10 uM in the composition. In some embodiments, each of the peptides are present in an amount of 25 uM in the composition. In some embodiments, each of the peptides are present in an amount of 50 uM in the composition. Any one of the aforementioned compositions or peptide combinations may be used in any one of the methods provided herein.

Modifications to a gluten peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc.

The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A gluten peptide may also be chemically modified at the level of amino acid side chains, of amino acid chirality, and/or of the peptide backbone.

Particular changes can be made to a gluten peptide to improve resistance to degradation or optimize solubility properties or otherwise improve bioavailability compared to the parent gluten peptide, thereby providing gluten peptides having similar or improved therapeutic, diagnostic and/or pharmacokinetic properties. A preferred such modification includes the use of an N-terminal acetyl group or pyroglutamate and/or a C-terminal amide. Such modifications have been shown in the art to significantly increase the half-life and bioavailability of the peptides compared to the parent peptides having a free N- and C-terminus (see, e.g., PCT Publication No.: WO/2010/060155). In some embodiments, a gluten peptide comprises an N-terminal acetyl group or pyroglutamate group, and/or a C-terminal amide group. In some embodiments of any one of the compositions or methods provided herein, the first, second and/or third peptides described above comprise an N-terminal acetyl group or pyroglutamate group, and/or a C-terminal amide group. In some embodiments of any one of the compositions or methods provided herein, the first, second, third, fourth, fifth, sixth, seventh, eighth, ninth, tenth, eleventh, twelfth, and/or thirteenth peptides described above comprise an N-terminal acetyl group or pyroglutamate group, and/or a C-terminal amide group. In some embodiments of any one of the compositions or methods provided herein, the second, fourth, fifth, sixth, seventh, eighth, ninth, tenth, eleventh, twelfth, thirteenth, fourteenth, fifteenth, and/or sixteenth peptides described above comprise an N-terminal acetyl group or pyroglutamate group, and/or a C-terminal amide group. In some embodiments of any one of the compositions or methods provided herein, the first, second, third, fourth, fifth, sixth, eighth, ninth, tenth, eleventh, twelfth, and/or thirteenth peptides described above comprise an N-terminal acetyl group or pyroglutamate group, and/or a C-terminal amide group. In some embodiments of any one of the compositions or methods provided herein, the second, fourth, fifth, sixth, eighth, ninth, tenth, eleventh, twelfth, thirteenth, fourteenth, fifteenth, and/or sixteenth peptides described above comprise an N-terminal acetyl group or pyroglutamate group, and/or a C-terminal amide group.

### Peptide Production

The peptides described herein (e.g., gluten peptides) can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides.

For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent.

### Gluten Challenge

In some embodiments, any one of the methods provided herein comprise a gluten challenge or a sample obtained from a subject before, during, or after a gluten challenge. Generally, a gluten challenge comprises administering to the subject a composition comprising wheat, rye, or barley, or one or more peptides thereof (e.g., a composition comprising a wheat gliadin, a rye secalin, or a barley hordein, or one or more peptides thereof), in some form for a defined period of time in order to activate the immune system of the subject, e.g., through activation of wheat-, rye- and/or barley-reactive T cells and/or mobilization of such T cells in the subject. Methods of gluten challenges are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L, Guandalini S; Gastroenterology. 2009; 137(6):1912-33. Celiac disease: from pathogenesis to novel therapies. Schuppan D, Junker Y, Barisani D; J Dent Res. 2008; 87(12):1100-1107. Orally based diagnosis of celiac disease: current perspectives. Pastore L, Campisi G, Compilato D, and Lo Muzio L; Gastroenterology. 2001; 120:636-651. Current Approaches to Diagnosis and Treatment of Celiac Disease: An Evolving Spectrum. Fasano A and Catassi C; Clin Exp Immunol. 2000; 120:38-45. Local challenge of oral mucosa with gliadin in patients with coeliac disease. Lahteenoja M, Maki M, Viander M, Toivanen A, Syrjanen S; Clin Exp Immunol. 2000; 120:10-11. The mouth-an accessible region for gluten challenge. Ellis H and Ciclitira P; Clinical Science. 2001; 101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005; 54:1217-1223. T cells in peripheral blood after gluten challenge in coeliac disease. Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). As described herein, it has been found that IL-2 levels were elevated in subjects with Celiac disease, even in the absence of a gluten challenge. Accordingly, in some embodiments, any one of the methods provided is performed on a sample from a subject who has not undergone a gluten challenge (e.g., been administered gluten for at least 3 days after a period of at least 1 week, 1 month, 1 year or more of being on a gluten-free diet) within 1 week, 2 weeks, 3 weeks, 4 weeks, or more of the sample being obtained from the subject. In other embodiments, any one of the methods provided herein comprises performing a gluten challenge on the subject or obtaining a sample from a subject before, during or after a gluten challenge, where the gluten challenge is for less than 3 days.

In some embodiments, the challenge comprises administering a composition comprising wheat, barley and/or rye, or one or more peptides thereof. In some embodiments, the wheat is wheat flour, the barely is barley flour, and the rye is rye flour. In some embodiments, the challenge comprises administering a composition comprising a wheat gliadin, a barley hordein and/or a rye secalin, or one or more peptides thereof, to the subject prior to determining a T cell response as described herein.

In some embodiments, the composition is administered to the subject more than once prior to determining the level of IL-2, and a sample is obtained from the subject after administration of the composition. In some embodiments, administration is daily for 1 or 2 days. In some embodiments, administration is more than once a day (e.g., twice a day) for 1 or 2 days. In some embodiments, the sample is obtained from the subject within 24 hours of administration of the composition. In some embodiments, the sample is obtained from the subject within 1, 2, 3, 4 or 5 days after administration of the composition. In some embodiments, the subject has been on a gluten-free diet for at least 4 weeks prior to commencing the gluten challenge.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a sample is obtained from a subject before, during, and/or after a gluten challenge as described herein.

### Other Testing

In some embodiments of any one of the methods provided, methods described herein further comprise other testing of a subject (e.g., based on the results of the methods described herein). As used herein, “other testing” describes use of at least one additional diagnostic method in addition to the methods provided herein. Any diagnostic method or combinations thereof for Celiac disease is contemplated as other testing. Exemplary other testing includes, but is not limited to, intestinal biopsy, serology (measuring the levels of one or more antibodies present in the serum), genotyping (see, e.g., Walker-Smith J A, et al. Arch Dis Child 1990), and measurement of a T cell response. Such other testing may be performed as part of the methods described herein or after the methods described herein (e.g., as a companion diagnostic), or before use of the methods described herein (e.g., as a first-pass screen to eliminate certain subjects before use of the methods described herein, e.g., eliminating those that do not have one or more HLA-DQA and HLA-DQB susceptibility alleles). In some embodiments of any one of the methods provided, no other testing is required to assess the subject's Celiac disease status, for example, having or not having Celiac disease.

Detection of serum antibodies (serology) is contemplated. The presence of such serum antibodies can be detected using methods known to those of skill in the art, e.g., by ELISA, histology, cytology, immunofluorescence or western blotting. Such antibodies include, but are not limited to: IgA anti-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

T cell response tests are also contemplated as other testing. In some embodiments, a T cell response test comprises contacting a sample comprising a T cell with at least one gluten peptide and measuring a T cell response in the sample. In some embodiments, a T cell response is measured by measuring a level of IFN-γ or IP-10, where an increased level of IFN-γ or IP-10 compared to a control level (e.g., a level of IFN-γ in a sample that has not been contacted with a gluten peptide) may identify a subject as having Celiac disease. T cell response tests are known in the art (see, e.g., PCT Publication Nos.: WO/2001/025793, WO/2003/104273, WO/2005/105129, and WO/2010/060155). Exemplary sequences for IFN-γ are provided herein. Exemplary sequences for IP-10 are provided below.

>gi|323422857|ref|NM_001565.3| Homo sapiens
chemokine (C-X-C motif) ligand 10 (CXCL10), mRNA
(SEQ ID NO: 866)
CTTTGCAGATAAATATGGCACACTAGCCCCACGTTTTCTGAGACATTCCT
CAATTGCTTAGACATATTCTGAGCCTACAGCAGAGGAACCTCCAGTCTCA
GCACCATGAATCAAACTGCCATTCTGATTTGCTGCCTTATCTTTCTGACT
CTAAGTGGCATTCAAGGAGTACCTCTCTCTAGAACTGTACGCTGTACCTG
CATCAGCATTAGTAATCAACCTGTTAATCCAAGGTCTTTAGAAAAACTTG
AAATTATTCCTGCAAGCCAATTTTGTCCACGTGTTGAGATCATTGCTACA
ATGAAAAAGAAGGGTGAGAAGAGATGTCTGAATCCAGAATCGAAGGCCAT
CAAGAATTTACTGAAAGCAGTTAGCAAGGAAAGGTCTAAAAGATCTCCTT
AAAACCAGAGGGGAGCAAAATCGATGCAGTGCTTCCAAGGATGGACCACA
CAGAGGCTGCCTCTCCCATCACTTCCCTACATGGAGTATATGTCAAGCCA
TAATTGTTCTTAGTTTGCAGTTACACTAAAAGGTGACCAATGATGGTCAC
CAAATCAGCTGCTACTACTCCTGTAGGAAGGTTAATGTTCATCATCCTAA
GCTATTCAGTAATAACTCTACCCTGGCACTATAATGTAAGCTCTACTGAG
GTGCTATGTTCTTAGTGGATGTTCTGACCCTGCTTCAAATATTTCCCTCA
CCTTTCCCATCTTCCAAGGGTACTAAGGAATCTTTCTGCTTTGGGGTTTA
TCAGAATTCTCAGAATCTCAAATAACTAAAAGGTATGCAATCAAATCTGC
TTTTTAAAGAATGCTCTTTACTTCATGGACTTCCACTGCCATCCTCCCAA
GGGGCCCAAATTCTTTCAGTGGCTACCTACATACAATTCCAAACACATAC
AGGAAGGTAGAAATATCTGAAAATGTATGTGTAAGTATTCTTATTTAATG
AAAGACTGTACAAAGTAGAAGTCTTAGATGTATATATTTCCTATATTGTT
TTCAGTGTACATGGAATAACATGTAATTAAGTACTATGTATCAATGAGTA
ACAGGAAAATTTTAAAAATACAGATAGATATATGCTCTGCATGTTACATA
AGATAAATGTGCTGAATGGTTTTCAAAATAAAAATGAGGTACTCTCCTGG
AAATATTAAGAAAGACTATCTAAATGTTGAAAGATCAAAAGGTTAATAAA
GTAATTATAACTAAGAAAAAAAAAAAA
>gi|149999382|ref|NP_001556.2| C-X-C motif
chemokine 10 precursor [Homo sapiens]
(SEQ ID NO: 867)
MNQTAILICCLIFLTLSGIQGVPLSRTVRCTCISISNQPVNPRSLEKLEI
IPASQFCPRVEITATMKKKGEKRCLNPESKAIKNLLKAVSKERSKRSP
>gi|149999382: 22-98 C-X-C motif chemokine
10 mature protein [Homo sapiens]
(SEQ ID NO: 868)
VPLSRTVRCTCISISNQPVNPRSLEKLEIIPASQFCPRVEIIATMKKKGE
KRCLNPESKAIKNLLKAVSKERSKRSP

### Treatment

In some embodiments of any one of the methods provided herein, the methods described herein further comprise a treatment step, such as treating a subject identified as having or likely as having Celiac disease. In some embodiments of any one of the methods provided, the methods comprise a step where information regarding treatment is provided to the subject. Such information can be given orally or in written form, such as with written materials. Written materials may be in an electronic form. Any known treatment of Celiac disease is contemplated herein. Exemplary treatments include, e.g., a gluten-free diet. Other exemplary treatments include endopeptidases, such as ALV003 (Alvine) and AT1001 (Alba), agents that inhibit transglutaminase activity, agents that block peptide presentation by HLA DQ2.5, or oral resins that bind to gluten peptides and reduce their bioavailability.

Compositions comprising gluten peptides for use in treating Celiac disease are known in the art (see, e.g., PCT Publication Nos.: WO/2001/025793, WO/2003/104273, WO/2005/105129, and WO/2010/060155, which are incorporated herein by reference in their entirety, including the gluten peptides in particular). In some embodiments, the composition comprises at least one of: (i) a first peptide comprising the amino acid sequence PFPQPELPY (SEQ ID NO: 869) and PQPELPYPQ (SEQ ID NO: 870), (ii) a second peptide comprising the amino acid sequence PFPQPEQPF (SEQ ID NO: 871) and PQPEQPFPW (SEQ ID NO: 872), or (iii) a third peptide comprising the amino acid sequence PIPEQPQPY (SEQ ID NO: 873). In some embodiments, the composition comprises the first and second peptide, the first and third peptide, or the second and third peptide. In some embodiments, the composition comprises the first and second peptide. In some embodiments, the composition comprises the first, second, and third peptide. In some embodiments, the first peptide comprises the amino acid sequence LQPFPQPELPYPQPQ (SEQ ID NO: 874); the second peptide comprises the amino acid sequence QPFPQPEQPFPWQP (SEQ ID NO: 875); and/or the third peptide comprises the amino acid sequence PEQPIPEQPQPYPQQ (SEQ ID NO: 876). Modifications to such peptides, e.g., an N-terminal pyro-glutamate and/or C-terminal amide, are contemplated and described herein. In some embodiments, the first peptide comprises the amino acid sequence ELQPFPQPELPYPQPQ (SEQ ID NO: 877), wherein the N-terminal glutamate is a pyroglutamate and the C-terminal glutamine is amidated; the second peptide comprises the amino acid sequence EQPFPQPEQPFPWQP (SEQ ID NO: 878), wherein the N-terminal glutamate is a pyroglutamate and the C-terminal proline is amidated (e.g., the free C-terminal COO is amidated); and/or the third peptide comprises the amino acid sequence EPEQPIPEQPQPYPQQ (SEQ ID NO: 879), wherein the N-terminal glutamate is a pyroglutamate and the C-terminal glutamine is amidated. In some embodiments, the first peptide consists of the amino acid sequence ELQPFPQPELPYPQPQ (SEQ ID NO: 880), wherein the N-terminal glutamate is a pyroglutamate and the C-terminal glutamine is amidated; the second peptide consists of the amino acid sequence EQPFPQPEQPFPWQP (SEQ ID NO: 881), wherein the N-terminal glutamate is a pyroglutamate and the C-terminal proline is amidated (e.g., the free C-terminal COO is amidated); and/or the third peptide consists of the amino acid sequence EPEQPIPEQPQPYPQQ (SEQ ID NO: 882), wherein the N-terminal glutamate is a pyroglutamate and the C-terminal glutamine is amidated (e.g., the free C-terminal COO is amidated). In some embodiments, the composition comprises 150 micrograms of the peptides (i.e., 50 micrograms of the first peptide and an equimolar amount of each of the second and third peptides). In some embodiments, the composition comprises 300 micrograms of the peptides (i.e., 100 micrograms of the first peptide and an equimolar amount of each of the second and third peptides). Any one of these compositions may be for use in any one of the methods or kits provided herein.

Treatments may be administered through any method known in the art. Pharmaceutical compositions suitable for each administration route are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005). In some embodiments, a treatment, e.g., a composition comprising a gluten peptide, such as those provided herein, is administered via injection, such as intradermal injection.

The peptides or other compositions provided herein may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. Compositions, such as pharmaceutical compositions, may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the pharmaceutical composition. The carrier can be any of those conventionally used and is limited only by chemico-physical considerations, such as solubility and lack of reactivity with the active agent, and by the route of administration. Suitable carriers for the pharmaceutical composition include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers. Other carriers are well known in the art (see, e.g., Remington's Pharmaceutical Sciences, 16th Ed. Mack Publishing Company, 1980 and Remington: The Science and Practice of Pharmacy, 21st Ed. Lippincott Williams & Wilkins, 2005).

The pharmaceutical composition(s) may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection. Examples of appropriate delivery mechanisms for intradermal administration include, but are not limited to, syringes, needles, and osmotic pumps.

It can be advantageous to formulate the active agent in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Alternatively, the compositions may be presented in multi-dose form. Examples of dosage units include sealed ampoules and vials and may be stored in a freeze-dried condition requiring only the addition of the sterile liquid carrier immediately prior to use.

The actual amount administered (or dose or dosage) and the rate and time-course of administration will depend on the nature and severity of the condition being treated as well as the characteristics of the subject to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the subject, the site of delivery, the method of administration and other factors known to practitioners. Examples of techniques and protocols can be found in, e.g., Remington's Pharmaceutical Sciences, 16th Ed. Mack Publishing Company, 1980 and Remington: The Science and Practice of Pharmacy, 21st Ed. Lippincott Williams & Wilkins, 2005. Effective amounts may be measured from ng/kg body weight to g/kg body weight per minute, hour, day, week or month.

As used herein, the terms “treat”, “treating”, and “treatment” include abrogating, inhibiting, slowing, or reversing the progression of a disease or condition, or ameliorating or preventing a clinical symptom of the disease (for example, Celiac disease). Treatment may include induction of immune tolerance (for example, to gluten or peptides thereof), modification of the cytokine secretion profile of the subject and/or induction of suppressor T cell subpopulations to secrete cytokines. Thus, a subject treated according to the disclosure preferably, in some embodiments, is able to eat at least wheat, rye, and/or barley without a significant T cell response which would normally lead to symptoms of Celiac disease. In some embodiments, an effective amount of a treatment is administered. The term “effective amount” means the amount of a treatment sufficient to provide the desired therapeutic or physiological effect when administered under appropriate or sufficient conditions.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Kits

Also disclosed herein are kits for measuring an immune response, e.g., by detecting IL-2 in a sample comprising a lymphocyte. In some embodiments, the kit comprises: (a) any one of the compositions comprising at least one gluten peptide as described herein and (b) a binding partner for IL-2. In some embodiments, the kit further comprises an agent that recognizes the binding partner for IL-2. In some embodiments, any one of the kits provided further comprises a container for blood. In some embodiments, the composition is contained within the container (e.g., dried onto the wall of the container).

In some embodiments of any one of the kits provided herein, the composition comprises at least one of:

(a) a first peptide comprising the amino acid sequence PFPQPELPY (SEQ ID NO: 883) and the amino acid sequence PQPELPYPQ (SEQ ID NO: 884);

(b) a second peptide comprising the amino acid sequence PFPQPEQPF (SEQ ID NO: 885) and the amino acid sequence PQPEQPFPW (SEQ ID NO: 886);

(c) a third peptide comprising the amino acid sequence EQPIPEQPQ (SEQ ID NO: 887) and the amino acid sequence PIPEQPQPY (SEQ ID NO: 888);

(d) a fourth peptide comprising the amino acid sequence PFPQPEQPI (SEQ ID NO: 889), the amino acid sequence PQPEQPIPV (SEQ ID NO: 890), and the amino acid sequence EQPIPVQPE (SEQ ID NO: 891);

(e) a fifth peptide comprising the amino acid sequence PFPQPEQPT (SEQ ID NO: 892), the amino acid sequence PQPEQPTPI (SEQ ID NO: 893), and the amino acid sequence EQPTPIQPE (SEQ ID NO: 894);

(f) a sixth peptide comprising the amino acid sequence PFPQPEQPF (SEQ ID NO: 895), the amino acid sequence PQPEQPFPL (SEQ ID NO: 896), and the amino acid sequence EQPFPLQPE (SEQ ID NO: 897);

(g) a seventh peptide comprising the amino acid sequence PFPQPEQPF (SEQ ID NO: 898) and the amino acid sequence PQPEQPFSQ (SEQ ID NO: 899);

(h) an eighth peptide comprising the amino acid sequence PYPEQPQPF (SEQ ID NO: 900);

(i) a ninth peptide comprising the amino acid sequence PFPEQPEQI (SEQ ID NO: 901);

(j) a tenth peptide comprising the amino acid sequence EGSFQPSQE (SEQ ID NO: 902);

(k) an eleventh peptide comprising the amino acid sequence QGYYPTSPQ (SEQ ID NO: 903);

(l) a twelfth peptide comprising the amino acid sequence EQPEQPFPE (SEQ ID NO: 904) and the amino acid sequence EQPFPEQPQ (SEQ ID NO: 905);

(m) a thirteenth peptide comprising the amino acid sequence PFSEQEQPV (SEQ ID NO: 906);

(n) a fourteenth peptide comprising the amino acid sequence EQPFPEQPI (SEQ ID NO: 907), the amino acid sequence PFPEQPIPE (SEQ ID NO: 908), the amino acid sequence EQPIPEQPQ (SEQ ID NO: 909), and the amino acid sequence PIPEQPQPY (SEQ ID NO: 910);

(o) a fifteenth peptide comprising the amino acid sequence PQPELPYPQ (SEQ ID NO: 911) and the amino acid sequence PYPQPELPY (SEQ ID NO: 912);

(p) a sixteenth peptide comprising the amino acid sequence PFPQPELPY (SEQ ID NO: 913) and the amino acid sequence PQPELPYPY (SEQ ID NO: 914);

(q) a seventeenth peptide comprising the amino acid sequence EQPFPEQPI (SEQ ID NO: 915) and the amino acid sequence PFPEQPIPE (SEQ ID NO: 916); and

(r) an eighteenth peptide comprising the amino acid sequence PQPYPEQPQ (SEQ ID NO: 917) and the amino acid sequence PYPEQPQPF (SEQ ID NO: 918).

In some embodiments,

(a) the first peptide comprises the amino acid sequence LQPFPQPELPYPQPQ (SEQ ID NO: 919);

(b) the second peptide comprises the amino acid sequence QPFPQPEQPFPWQP (SEQ ID NO: 920);

(c) the third peptide comprises the amino acid sequence PEQPIPEQPQPYPQQ (SEQ ID NO: 921);

(d) the fourth peptide comprises the amino acid sequence QPFPQPEQPIPVQPEQS (SEQ ID NO: 922);

(e) the fifth peptide comprises the amino acid sequence QPFPQPEQPTPIQPEQP (SEQ ID NO: 923);

(f) the sixth peptide comprises the amino acid sequence QPFPQPEQPFPLQPEQP (SEQ ID NO: 924);

(g) the seventh peptide comprises the amino acid sequence QPFPQPEQPFSQQ (SEQ ID NO: 925);

(h) the eighth peptide comprises the amino acid sequence PQPYPEQPQPFPQQ (SEQ ID NO: 926);

(i) the ninth peptide comprises the amino acid sequence QPFPEQPEQIIPQQP (SEQ ID NO: 927);

(j) the tenth peptide comprises the amino acid sequence SGEGSFQPSQENPQ (SEQ ID NO: 928);

(k) the eleventh peptide comprises the amino acid sequence GQQGYYPTSPQQSG (SEQ ID NO: 929);

(l) the twelfth peptide comprises the amino acid sequence PEQPEQPFPEQPQQ (SEQ ID NO: 930);

(m) the thirteenth peptide comprises the amino acid sequence QPPFSEQEQPVLPQ (SEQ ID NO: 931);

(n) the fourteenth peptide comprises the amino acid sequence PEQPFPEQPIPEQPQPYP (SEQ ID NO: 932);

(o) the fifteenth peptide comprises the amino acid sequence QPYPQPELPYPQPQ (SEQ ID NO: 933);

(p) the sixteenth peptide comprises the amino acid sequence QPFPQPELPYPYPQ (SEQ ID NO: 934);

(q) the seventeenth peptide comprises the amino acid sequence PQEQPFPEQPIPEQP (SEQ ID NO: 935); and

(r) the eighteenth peptide comprises the amino acid sequence QPQPYPEQPQPFPQQ (SEQ ID NO: 936).

In some embodiments of any one of the kits provided herein, the composition comprises at least one of:

(i) a first peptide comprising the amino acid sequence PFPQPELPY (SEQ ID NO: 937) and the amino acid sequence PQPELPYPQ (SEQ ID NO: 938);

(ii) a second peptide comprising the amino acid sequence PFPQPEQPF (SEQ ID NO: 939) and the amino acid sequence PQPEQPFPW (SEQ ID NO: 940);

(iii) a third peptide comprising the amino acid sequence PIPEQPQPY (SEQ ID NO: 941);

(iv) a fourth peptide comprising the amino acid sequence PFPQPEQPIP (SEQ ID NO: 942) and the amino acid sequence EQPIPVQPE (SEQ ID NO: 943);

(v) a fifth peptide comprising the amino acid sequence PFPQPEQPTPI (SEQ ID NO: 944) and the amino acid sequence EQPTPIQPE (SEQ ID NO: 945);

(vi) a sixth peptide comprising the amino acid sequence PQPEQPFPL (SEQ ID NO: 946) and the amino acid sequence EQPFPLQPE (SEQ ID NO: 947);

(vii) a seventh peptide comprising the amino acid sequence PFPQPEQPF (SEQ ID NO: 948) and the amino acid sequence PQPEQPFSQ (SEQ ID NO: 949);

(viii) an eighth peptide comprising the amino acid sequence PYPEQPQPF (SEQ ID NO: 950);

(ix) a ninth peptide comprising the amino acid sequence PFPEQPEQIIP (SEQ ID NO: 951);

(x) a tenth peptide comprising the amino acid sequence EGSFQPSQE (SEQ ID NO: 952);

(xi) an eleventh peptide comprising the amino acid sequence QGYYPTSPQ (SEQ ID NO: 953);

(xii) a twelfth peptide comprising the amino acid sequence EQPEQPFPEQPQ (SEQ ID NO: 954);

(xiii) a thirteenth peptide comprising the amino acid sequence PFSEQEQPV (SEQ ID NO: 955);

(xiv) a fourteenth peptide comprising the amino acid sequence EQPFPEQPI (SEQ ID NO: 956) and PIPEQPQPY (SEQ ID NO: 957);

(xv) a fifteenth peptide comprising the amino acid sequence PQPELPYPQ (SEQ ID NO: 958) and the amino acid sequence PYPQPELPY (SEQ ID NO: 959);

(xvi) a sixteenth peptide comprising the amino acid sequence PFPQPELPY (SEQ ID NO: 960) and the amino acid sequence PQPELPYPY (SEQ ID NO: 961); and

(xvii) a seventeenth peptide comprising the amino acid sequence EQPFPEQPI (SEQ ID NO: 962).

In some embodiments,

(i) the first peptide comprises the amino acid sequence LQPFPQPELPYPQPQ (SEQ ID NO: 963);

(ii) the second peptide comprises the amino acid sequence QPFPQPEQPFPWQP; (SEQ ID NO: 964)

(iii) the third peptide comprises the amino acid sequence PEQPIPEQPQPYPQQ (SEQ ID NO: 965);

(iv) the fourth peptide comprises the amino acid sequence QPFPQPEQPIPVQPEQS (SEQ ID NO: 966);

(v) the fifth peptide comprises the amino acid sequence QPFPQPEQPTPIQPEQP (SEQ ID NO: 967);

(vi) the sixth peptide comprises the amino acid sequence QPFPQPEQPFPLQPEQP (SEQ ID NO: 968);

(vii) the seventh peptide comprises the amino acid sequence QPFPQPEQPFSQQ (SEQ ID NO: 969);

(viii) the eighth peptide comprises the amino acid sequence PQPYPEQPQPFPQQ (SEQ ID NO: 970);

(ix) the ninth peptide comprises the amino acid sequence QPFPEQPEQIIPQQP (SEQ ID NO: 971);

(x) the tenth peptide comprises the amino acid sequence SGEGSFQPSQENPQ (SEQ ID NO: 972);

(xi) the eleventh peptide comprises the amino acid sequence GQQGYYPTSPQQSG (SEQ ID NO: 973);

(xii) the twelfth peptide comprises the amino acid sequence PEQPEQPFPEQPQQ (SEQ ID NO: 974);

(xiii) the thirteenth peptide comprises the amino acid sequence QPPFSEQEQPVLPQ (SEQ ID NO: 975);

(xiv) the fourteenth peptide comprises the amino acid sequence PEQPFPEQPIPEQPQPYP (SEQ ID NO: 976);

(xv) the fifteenth peptide comprises the amino acid sequence QPYPQPELPYPQPQ (SEQ ID NO: 977);

(xvi) the sixteenth peptide comprises the amino acid sequence QPFPQPELPYPYPQ (SEQ ID NO: 978); and

(xvii) the seventeenth peptide comprises the amino acid sequence EQPFPEQPI (SEQ ID NO: 979). In some embodiments, the composition comprises the first, second, and third peptides. In some embodiments, the composition comprises the first, second, third, fourth, fifth, sixth, seventh, eighth, ninth, tenth, eleventh, twelfth, and thirteenth peptides. In some embodiments, the composition comprises the second, fourth, fifth, sixth, seventh, eighth, ninth, tenth, eleventh, twelfth, thirteenth, fourteenth, fifteenth, and sixteenth peptides. In some embodiments, the composition comprises the first, second, third, fourth, fifth, sixth, eighth, ninth, tenth, eleventh, twelfth, and thirteenth peptides. In some embodiments, the composition comprises the second, fourth, fifth, sixth, eighth, ninth, tenth, eleventh, twelfth, thirteenth, fourteenth, fifteenth, and sixteenth peptides. In some embodiments, the composition comprises the second, third, fourth, fifth, sixth, seventh, eighth, ninth, tenth, eleventh, twelfth, thirteenth, fifteenth, sixteenth, and seventeenth peptides. In some embodiments, the composition comprises the second, third, fourth, fifth, sixth, eighth, ninth, tenth, eleventh, twelfth, thirteenth, fifteenth, sixteenth, and seventeenth peptides. In some embodiments, at least one of the peptides comprises an N-terminal pyroglutamate and/or a C-terminal amide group. In some embodiments, each of the peptides comprises an N-terminal pyroglutamate and/or a C-terminal amide group.

In some embodiments of any one of the kits provided herein, the composition comprises at least one of:

(a) a first peptide comprising the amino acid sequence PFPQPELPY (SEQ ID NO: 980) and the amino acid sequence PQPELPYPQ (SEQ ID NO: 981);

(b) a second peptide comprising the amino acid sequence PFPQPEQPF (SEQ ID NO: 982) and the amino acid sequence PQPEQPFPW (SEQ ID NO: 983);

(c) a third peptide comprising the amino acid sequence EQPIPEQPQ (SEQ ID NO: 984) and the amino acid sequence PIPEQPQPY (SEQ ID NO: 985);

(d) a fourth peptide comprising the amino acid sequence PFPQPEQPI (SEQ ID NO: 986) and the amino acid sequence PQPEQPIPV (SEQ ID NO: 987);

(e) a fifth peptide comprising the amino acid sequence EQPIPVQPE (SEQ ID NO: 988);

(f) a sixth peptide comprising the amino acid sequence PFPQPEQPT (SEQ ID NO: 989) and the amino acid sequence PQPEQPTPI (SEQ ID NO: 990);

(g) a seventh peptide comprising the amino acid sequence EQPTPIQPE (SEQ ID NO: 991);

(h) an eighth peptide comprising the amino acid sequence PQPEQPFPL (SEQ ID NO: 992);

(i) a ninth peptide comprising the amino acid sequence EQPFPLQPE (SEQ ID NO: 993);

(j) a tenth peptide comprising the amino acid sequence EGSFQPSQE (SEQ ID NO: 994);

(k) a eleventh peptide comprising the amino acid sequence QGYYPTSPQ (SEQ ID NO: 995);

(l) a twelfth peptide comprising the amino acid sequence EQPEQPFPE (SEQ ID NO: 996);

(m) a thirteenth peptide comprising the amino acid sequence PFSEQEQPV (SEQ ID NO: 997);

(n) a fourteenth peptide comprising the amino acid sequence PYPQPELPY (SEQ ID NO: 998);

(o) a fifteenth peptide comprising the amino acid sequence EQPFPEQPI (SEQ ID NO: 999) and the amino acid sequence PFPEQPIPE (SEQ ID NO: 1000); and

(p) a sixteenth peptide comprising the amino acid sequence PYPEQPQPF (SEQ ID NO: 1001) and the amino acid sequence PQPYPEQPQ (SEQ ID NO: 1002).

In some embodiments:

(a) the first peptide comprises the amino acid sequence PFPQPELPYPQP (SEQ ID NO: 1003);

(b) the second peptide comprises the amino acid sequence PFPQPEQPFPWQ (SEQ ID NO: 1004);

(c) the third peptide comprises the amino acid sequence EQPIPEQPQPYP (SEQ ID NO: 1005);

(d) the fourth peptide comprises the amino acid sequence PFPQPEQPIPVQ (SEQ ID NO: 1006);

(e) the fifth peptide comprises the amino acid sequence PEQPIPVQPEQS (SEQ ID NO: 1007);

(f) the sixth peptide comprises the amino acid sequence PFPQPEQPTPIQ (SEQ ID NO: 1008);

(g) the seventh peptide comprises the amino acid sequence PEQPTPIQPEQP (SEQ ID NO: 1009);

(h) the eighth peptide comprises the amino acid sequence PFPQPEQPFPLQ (SEQ ID NO: 1010);

(i) the ninth peptide comprises the amino acid sequence PEQPFPLQPEQP (SEQ ID NO: 1011);

(j) the tenth peptide comprises the amino acid sequence GEGSFQPSQENP (SEQ ID NO: 1012);

(k) the eleventh peptide comprises the amino acid sequence QQGYYPTSPQQS (SEQ ID NO: 1013);

(l) the twelfth peptide comprises the amino acid sequence PEQPEQPFPEQP (SEQ ID NO: 1014);

(m) the thirteenth peptide comprises the amino acid sequence PPFSEQEQPVLP (SEQ ID NO: 1015);

(n) the fourteenth peptide comprises the amino acid sequence PYPQPELPYPQP (SEQ ID NO: 1016);

(o) the fifteenth peptide comprises the amino acid sequence EQPFPEQPIPEQ (SEQ ID NO: 1017); and

(p) the sixteenth peptide comprises the amino acid sequence PQPYPEQPQPFP (SEQ ID NO: 1018).

In some embodiments of any one of the kits provided herein, the composition comprises at least four (e.g., five, six, seven, eight, nine, ten, eleven, twelve, thirteen, fourteen, fifteen or sixteen) of the peptides. In some embodiments of any one of the kits provided herein, the composition comprises (or consists of) the peptides in (a)-(p). In some embodiments of any one of the kits provided herein, at least one of the peptides comprises an N-terminal pyroglutamate and/or a C-terminal amide group. In some embodiments of any one of the kits provided herein, each of the peptides comprises an N-terminal pyroglutamate and/or a C-terminal amide group.

In some embodiments of any one of the kits provided herein, each of the peptides are present in an amount of 2.5 ug/mL in the composition. In some embodiments of any one of the kits provided herein, each of the peptides are present in an amount of 5 ug/mL in the composition. In some embodiments of any one of the kits provided herein, each of the peptides are present in an amount of 10 ug/mL in the composition. In some embodiments of any one of the kits provided herein, each of the peptides are present in an amount of 20 ug/mL in the composition. In some embodiments of any one of the kits provided herein, each of the peptides are present in an amount of 25 ug/mL in the composition. In some embodiments of any one of the kits provided herein, each of the peptides are present in an amount of 50 ug/mL in the composition. In some embodiments of any one of the kits provided herein, each of the peptides are present in an amount of 5 uM in the composition. In some embodiments of any one of the kits provided herein, each of the peptides are present in an amount of 10 uM in the composition. In some embodiments of any one of the kits provided herein, each of the peptides are present in an amount of 25 uM in the composition. In some embodiments of any one of the kits provided, each of the peptides are present in an amount of 50 uM in the composition.

In some embodiments of any one of the kits provided herein, the kit further comprises a binding partner for IP-10 or IFN-γ.

Any suitable binding partner for IL-2 (or IP-10 or IFN-γ) is contemplated. In some embodiments, the binding partner is any molecule that binds specifically to an IL-2 protein (or IP-10 protein or IFN-γ protein). As described herein, “binds specifically to a protein” means that the molecule is more likely to bind to a portion of or the entirety of the protein than to a portion of or the entirety of another protein. In some embodiments, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically to IL-2. Methods for producing peptide molecules and aptamers are well known in the art (see, e.g., published US Patent Application No. 2009/0075834, U.S. Pat. Nos. 7,435,542, 7,807,351, and 7,239,742). In some embodiments, the binding partner is any molecule that binds specifically to an IL-2 mRNA (or IP-10 mRNA or IFN-γ mRNA). As described herein, “binds specifically to an mRNA” means that the molecule is more likely to bind to a portion of or the entirety of the mRNA (e.g., by complementary base-pairing) than to a portion of or the entirety of another mRNA or other nucleic acid. In some embodiments, the binding partner that binds specifically to the mRNA is a nucleic acid, e.g., a probe. Binding partners can be designed using the nucleotide and amino acid sequences of IL-2, IP-10 and IFN-γ provided herein. In some embodiments, the binding partner for IL-2 is an anti-IL-2 antibody or an antigen-binding fragment thereof. In some embodiments, the binding partner for IFN-γ or IP-10 is an anti-IFN-γ or anti-IP-10 antibody or an antigen-binding fragment thereof.

In some embodiments, any one of the kits provided comprises a first and second binding partner for IL-2. In some embodiments, the first and second binding partners are antibodies or antigen binding fragments thereof. In some embodiments, the second binding partner is bound to a surface. The second binding partner may be bound to the surface covalently or non-covalently. The second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments, the first binding partner for IL-2 is washed over the IL-2 bound to the second binding partner (e.g., as in a sandwich ELISA). The first binding partner may comprise a detectable label, or an agent that recognizes the first binding partner for IL-2 (e.g., a secondary antibody) may comprise a detectable label.

Any suitable agent that recognizes a binding partner for IL-2 (or IP-10 or IFN-γ) is contemplated. In some embodiments, the binding partner is any molecule that binds specifically to the binding partner for IL-2 (or IP-10 or IFN-γ). In some embodiments, the agent is an antibody (e.g., a secondary antibody) or antigen-binding fragment thereof, such as Fab, F(ab)2, Fv, single chain antibodies, Fab and sFab fragments, F(ab′)2, Fd fragments, scFv, or dAb fragments. Agents also include other peptide molecules and aptamers that bind specifically to a binding partner for IL-2 (or IP-10 or IFN-γ). In some embodiments, the binding partner for IL-2 comprises a biotin moiety and the agent is a composition that binds to the biotin moiety (e.g., an avidin or streptavidin).

In some embodiments of any one of the kits provided, the binding partner for IL-2 (or IP-10 or IFN-γ) and/or the agent comprise a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art are detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments of any one of the kits provided, the kit further comprises a negative control, e.g., a composition that does not comprise a gluten peptide, e.g., a saline solution or cell culture medium. In some embodiments of any one of the kits provided, the kit further comprises a positive control, e.g., a composition comprising IL-2 at a known concentration.

In some embodiments of any one of the kits provided, the kit comprises any combination of the components mentioned above.

In some embodiments of any one of the kits provided, the kit further comprises instructions for detecting IL-2 in a sample from a subject suspected of having Celiac disease. In some embodiments of any one of the kits provided, the instructions include any one of the methods as described herein. Instructions can be in any suitable form, e.g., as a printed insert or a label.

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Sambrook et al., Molecular Cloning: A Laboratory Manual, Cold Spring Harbour Laboratory Press (2012); T. A. Brown (editor), Essential Molecular Biology: A Practical Approach, Volumes 1 and 2, IRL Press (2000 and 2002); D. M. Glover and B. D. Hames (editors), Current Protocols in Molecular Biology, Greene Pub. Associates and Wiley-Interscience (1988, including all updates until present); Edward A. Greenfield (editor) Antibodies: A Laboratory Manual, Cold Spring Harbour Laboratory, (2013); and J. E. Coligan et al. (editors), Current Protocols in Immunology, John Wiley & Sons (including all updates until present).

In any one aspect or embodiment provided herein “comprising” may be replaced with “consisting essentially of” or “consisting of”.

### EXAMPLES

### Example 1

### Methods

HLA-DQ2.5-positive celiac disease subjects on gluten-free diet are used in this study. Blood is collected immediately before and 6 days after commencing 3-day oral gluten challenge. Whole blood or PBMCs are incubated with pools or single peptides derived from gluten or recall antigens. IL-2 levels are measured in plasma from the whole blood that was incubated in 96-well plates with peptides or peptide pools. Plasma levels are measured by MAGPIX® multiplex bead assay or by ELISA. Alternatively, IL-2 levels are measured in PBMCs separated from the blood sample are incubated in overnight IL-2 ELISpot assays.

The peptide pools used are:

| P3 pool (pE = pyroglutamate) | |
|---|---|
| (pE)LQPFPQPELPYPQPQ-amide | (SEQ ID NO: 1019) |
| (pE)QPFPQPEQPFPWQP-amide | (SEQ ID NO: 1020) |
| (pE)PEQPIPEQPQPYPQQ-amide | (SEQ ID NO: 1021) |
| P13 pool (pE = pyroglutamate) | |
| (pE)LQPFPQPELPYPQPQ-amide | (SEQ ID NO: 1022) |
| (pE)QPFPQPEQPFPWQP-amide | (SEQ ID NO: 1023) |
| (pE)PEQPIPEQPQPYPQQ-amide | (SEQ ID NO: 1024) |
| (pE)QPFPQPEQPIPVQPEQS-amide | (SEQ ID NO: 1025) |
| (pE)QPFPQPEQPTPIQPEQP-amide | (SEQ ID NO: 1026) |
| (pE)QPFPQPEQPFPLQPEQP-amide | (SEQ ID NO: 1027) |
| (pE)QPFPQPEQPFSQQ-amide | (SEQ ID NO: 1028) |
| (pE)PQPYPEQPQPFPQQ-amide | (SEQ ID NO: 1029) |
| (pE)QPFPEQPEQIIPQQP-amide | (SEQ ID NO: 1030) |
| (pE)SGEGSFQPSQENPQ-amide | (SEQ ID NO: 1031) |
| (pE)GQQGYYPTSPQQSG-amide | (SEQ ID NO: 1032) |
| (pE)PEQPEQPFPEQPQQ-amide | (SEQ ID NO: 1033) |
| (pE)QPPFSEQEQPVLPQ-amide | (SEQ ID NO: 1034) |
| P14 pool (pE = pyroglutamate) | |
| (pE)QPFPQPEQPFPWQP-amide | (SEQ ID NO: 1035) |
| (pE)QPFPQPEQPIPVQPEQS-amide | (SEQ ID NO: 1036) |
| (pE)QPFPQPEQPTPIQPEQP-amide | (SEQ ID NO: 1037) |
| (pE)QPFPQPEQPFPLQPEQP-amide | (SEQ ID NO: 1038) |
| (pE)QPFPQPEQPFSQQ-amide | (SEQ ID NO: 1039) |
| (pE)PQPYPEQPQPFPQQ-amide | (SEQ ID NO: 1040) |
| (pE)QPFPEQPEQIIPQQP-amide | (SEQ ID NO: 1041) |
| (pE)SGEGSFQPSQENPQ-amide | (SEQ ID NO: 1042) |
| (pE)GQQGYYPTSPQQSG-amide | (SEQ ID NO: 1043) |
| (pE)PEQPEQPFPEQPQQ-amide | (SEQ ID NO: 1044) |
| (pE)QPPFSEQEQPVLPQ-amide | (SEQ ID NO: 1045) |
| (pE)PEQPFPEQPIPEQPQPYP-amide | (SEQ ID NO: 1046) |
| (pE)QPYPQPELPYPQPQ-amide | (SEQ ID NO: 1047) |
| (pE)QPFPQPELPYPYPQ-amide | (SEQ ID NO: 1048) |

Each peptide in the above pools is designed to include at least one T cell epitope. The peptide pools are provided such that equimolar amounts of each peptide were present in each pool. A total gluten pool including 71 peptides capturing the majority of T cell epitopes in gluten is used as a control to simulate total gluten.

The peptide pools are further described in the table below, including exemplary epitope sequences. P13alt pool is another exemplary pool for use in this Example.

TABLE 1
Peptide Pools

| Pep- | P3 | P13 | P14 | P13alt | | HLA-DQ | Epitope |
|---|---|---|---|---|---|---|---|
| tide | pool | pool | pool | pool | Sequence | Restriction | sequences |
| 1 | Present | Present | Absent | Present | (pE)LQPFPQPE | DQ2.5 | PFPQPELPY |
| | | | | | LPYPQPQ- | | (SEQ ID |
| | | | | | amide | | NO: 1067), |
| | | | | | (SEQ ID | | PQPELPYPQ |
| | | | | | NO: 1049) | | (SEQ ID |
| | | | | | | | NO: 1068) |
| 2 | Present | Present | Present | Present | (pE)QPFPQPEQ | DQ2.5 | PFPQPEQPF |
| | | | | | PFPWQP-amide | | (SEQ ID |
| | | | | | (SEQ ID | | NO: 1069), |
| | | | | | NO: 1050) | | PQPEQPFPW |
| | | | | | | | (SEQ ID |
| | | | | | | | NO: 1070) |
| 3 | Present | Present | Absent | Present | (pE)PEQPIPEQP | DQ2.5 | EQPIPEQPQ |
| | | | | | QPYPQQ-amide | | (SEQ ID |
| | | | | | (SEQ ID | | NO: 1071), |
| | | | | | NO: 1051) | | PIPEQPQPY |
| | | | | | | | (SEQ ID |
| | | | | | | | NO: 1072) |
| 4 | Absent | Present | Present | Present | (pE)QPFPQPEQ | DQ2.5/2.5 + | PFPQPEQPI |
| | | | | | PIPVQPEQS- | 8/8 | (SEQ ID |
| | | | | | amide | | NO: 1073), |
| | | | | | (SEQ ID | | PQPEQPIPV |
| | | | | | NO: 1052) | | (SEQ ID |
| | | | | | | | NO: 1074), |
| | | | | | | | EQPIPVQPE |
| | | | | | | | (SEQ ID |
| | | | | | | | NO: 1075) |
| 5 | Absent | Present | Present | Present | (pE)QPFPQPEQ | DQ2.5/2.5 + | PFPQPEQPT |
| | | | | | PTPIQPEQP- | 8/8 | (SEQ ID |
| | | | | | amide | | NO: 1076), |
| | | | | | (SEQ ID | | PQPEQPTPI |
| | | | | | NO: 1053) | | (SEQ ID |
| | | | | | | | NO: 1077), |
| | | | | | | | EQPTPIQPE |
| | | | | | | | (SEQ ID |
| | | | | | | | NO: 1078) |
| 6 | Absent | Present | Present | Present | (pE)QPFPQPEQ | DQ2.5/2.5 + | PFPQPEQPF |
| | | | | | PFPLQPEQP- | 8/8 | (SEQ ID |
| | | | | | amide | | NO: 1079), |
| | | | | | (SEQ ID | | PQPEQPFPL |
| | | | | | NO: 1054) | | (SEQ ID |
| | | | | | | | NO: 1080), |
| | | | | | | | EQPFPLQPE |
| | | | | | | | (SEQ ID |
| | | | | | | | NO: 1081) |
| 7 | Absent | Present | Present | Absent | (pE)QPFPQPEQ | DQ2.5 | PFPQPEQPF |
| | | | | | PFSQQ-amide | | (SEQ ID |
| | | | | | (SEQ ID | | NO: 1082), |
| | | | | | NO: 1055) | | PQPEQPFSQ |
| | | | | | | | (SEQ ID |
| | | | | | | | NO: 1083) |
| 8 | Absent | Present | Present | Absent | (pE)PQPYPEQP | DQ2.5 | PYPEQPQPF |
| | | | | | QPFPQQ-amide | | (SEQ ID |
| | | | | | (SEQ ID | | NO: 1084) |
| | | | | | NO: 1056) | | |
| 9 | Absent | Present | Present | Present | (pE)SGEGSFQP | DQ8/2.5 + | EGSFQPSQE |
| | | | | | SQENPQ-amide | 8/8 | (SEQ ID |
| | | | | | (SEQ ID | | NO: 1085) |
| | | | | | NO: 1057) | | |
| 10 | Absent | Present | Present | Present | (pE)GQQGYYP | DQ2.5/2.5 + | QGYYPTSPQ |
| | | | | | TSPQQSG- | 8/8 | (SEQ ID |
| | | | | | amide | | NO: 1086) |
| | | | | | (SEQ ID | | |
| | | | | | NO: 1058) | | |
| 11 | Absent | Present | Present | Present | (pE)PEQPEQPF | DQ2.5/2.5 + | EQPEQPFPE |
| | | | | | PEQPQQ-amide | 8/8/2.2 + 8 | (SEQ ID |
| | | | | | (SEQ ID | | NO: 1087), |
| | | | | | NO: 1059) | | EQPFPEQPQ |
| | | | | | | | (SEQ ID |
| | | | | | | | NO: 1088) |
| 12 | Absent | Present | Present | Absent | (pE)QPFPEQPE | DQ2.5 | PFPEQPEQI |
| | | | | | QIIPQQP-amide | | (SEQ ID |
| | | | | | (SEQ ID | | NO: 1089) |
| | | | | | NO: 1060) | | |
| 13 | Absent | Present | Present | Present | (pE)QPPFSEQE | DQ2.2 | PFSEQEQPV |
| | | | | | QPVLPQ-amide | | (SEQ ID |
| | | | | | (SEQ ID | | NO: 1090) |
| | | | | | NO: 1061) | | |
| 14 | Absent | Absent | Present | Absent | (pE)PEQPFPEQ | DQ2.5 | EQPFPEQPI |
| | | | | | PIPEQPQPYP- | | (SEQ ID |
| | | | | | amide | | NO: 1091), |
| | | | | | (SEQ ID | | PFPEQPIPE |
| | | | | | NO: 1062) | | (SEQ ID |
| | | | | | | | NO: 1092), |
| | | | | | | | EQPIPEQPQ |
| | | | | | | | (SEQ ID |
| | | | | | | | NO: 1093), |
| | | | | | | | PIPEQPQPY |
| | | | | | | | (SEQ ID |
| | | | | | | | NO: 1094) |
| 15 | Absent | Absent | Present | Present | (pE)QPYPQPEL | DQ2.5 | PYPQPELPY |
| | | | | | PYPQPQ-amide | | (SEQ ID |
| | | | | | (SEQ ID | | NO: 1095), |
| | | | | | NO: 1063) | | PQPELPYPQ |
| | | | | | | | (SEQ ID |
| | | | | | | | NO: 1096) |
| 16 | Absent | Absent | Present | Absent | (pE)QPFPQPEL | DQ2.5 | PFPQPELPY |
| | | | | | PYPYPQ-amide | | (SEQ ID |
| | | | | | (SEQ ID | | NO: 1097), |
| | | | | | NO: 1064) | | PQPELPYPY |
| | | | | | | | (SEQ ID |
| | | | | | | | NO: 1098) |
| 17 | Absent | Absent | Absent | Present | (pE)PQEQPFPE | DQ2.5 | EQPFPEQPI |
| | | | | | QPIPEQP-amide | | (SEQ ID |
| | | | | | (SEQ ID | | NO: 1099), |
| | | | | | NO: 1065) | | PFPEQPIPE |
| | | | | | | | (SEQ ID |
| | | | | | | | NO: 1100) |
| 18 | Absent | Absent | Absent | Present | (pE)QPQPYPEQ | DQ2.5 | PQPYPEQPQ |
| | | | | | PQPFPQQ-amide | | (SEQ ID |
| | | | | | (SEQ ID | | NO: 1101), |
| | | | | | NO: 1066) | | PYPEQPQPF |
| | | | | | | | (SEQ ID |
| | | | | | | | NO: 1102) |

Peptide = Peptide identifier, (pE) = pyroglutamate, Present = present in the pool listed in the top row (P3, P13, P14, or P13alt), Absent = not present in the pool listed in the top row (P3, P13, P14, P13alt).

### Example 2

Blood samples were collected from HLA-DQ2.5+ volunteers with Celiac disease usually following strict gluten free diet. Blood (1 mL per tube) was drawn into sterile Quantiferon-Gold TB NIL tubes. Either 0.1 mL phosphate buffered saline (PBS) or 0.1 mL PBS containing peptide pool 1 (each of the 3 peptides present at 0.5 mg/mL) was added by injection through the cap of the tube.

Peptide pool 1 - 3 peptides (pE = pyroglutamate)

| | |
|---|---|
| (pE)LQPFPQPELPYPQPQ-amide | (SEQ ID NO: 1103) |
| (pE)QPFPQPEQPFPWQP-amide | (SEQ ID NO: 1104) |
| (pE)PEQPIPEQPQPYPQQ-amide | (SEQ ID NO: 1105) |

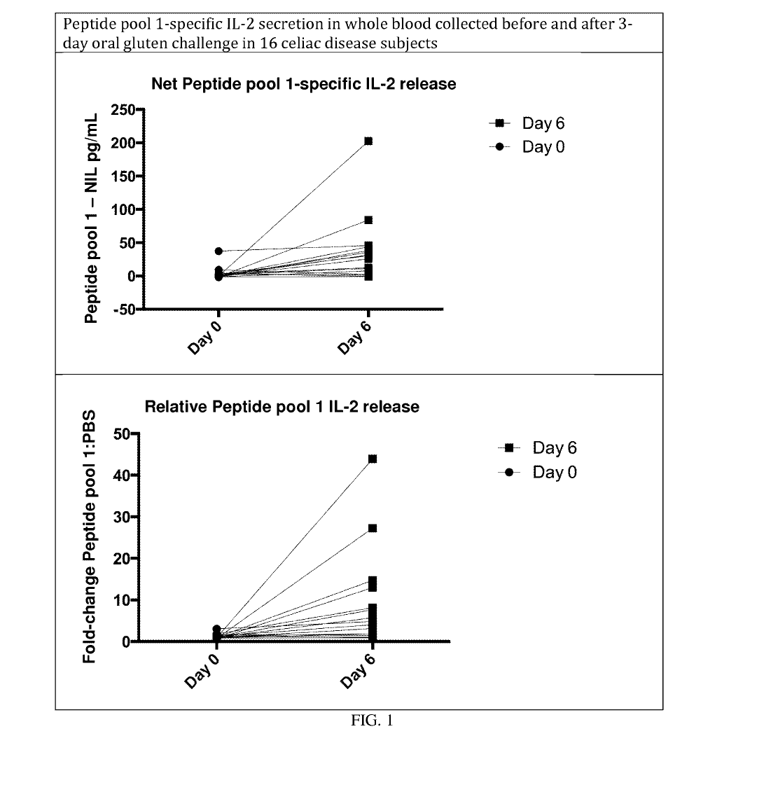

Tubes were incubated at 37 degrees C. for 24 h before plasma was separated and frozen at −80 degrees C. Blood was again collected and processed in the same manner from the same patients six days after commencing an oral gluten challenge. The oral challenge consisted of 3 cookies consumed daily that together provided approximately 9 grams of gluten daily. Cytokine levels in thawed plasma samples were assessed by Luminex 38plex magnetic bead assay. Several cytokines were elevated after incubation of blood with Peptide pool 1, particularly in blood collected after oral challenge. The plasma concentration of IL-2 in the Peptide pool 1 tube after subtraction of the PBS control tube, were higher on Day-6 than on Day-0 in 12/16 subjects (p<0.0008 Wilcoxon paired rank sum, FIG. 1). Median “net plasma levels” of IL-2 on Day-0 were 0 pg/mL compared to 28 pg/mL on Day-6. The median fold difference in IL-2 plasma concentration between the Peptide pool 1 tube and PBS tube rose from 1.0 on Day-0 to 4.4 on Day-6 (p<0.001 Wilcoxon paired rank sum, FIG. 1). According to the cutoffs set at 3 pg/mL and 1.4 fold-difference between Peptide pool 1 tube and PBS tube, 12 of 16 subjects were considered “positive” for IL-2 on Day-6.

### Example 3

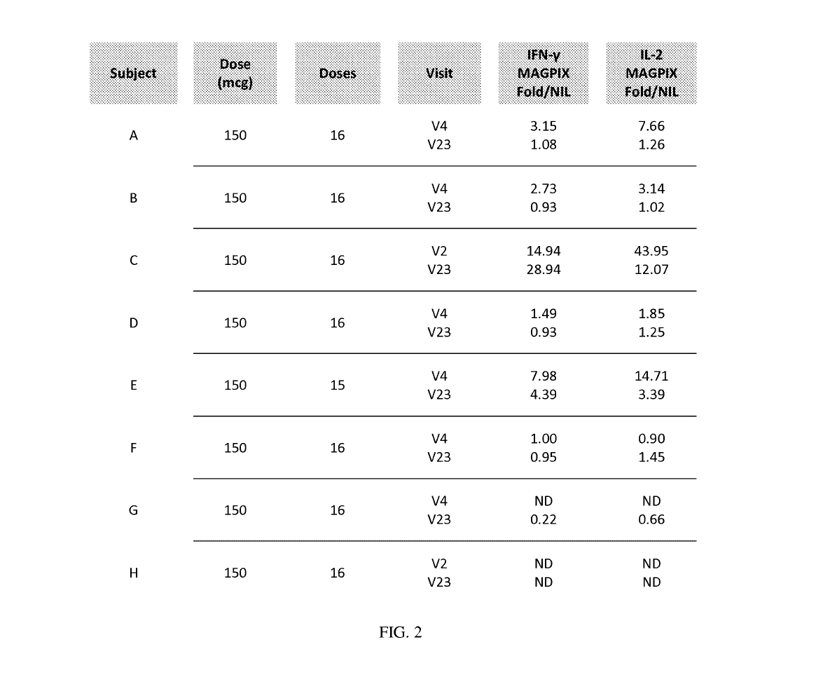

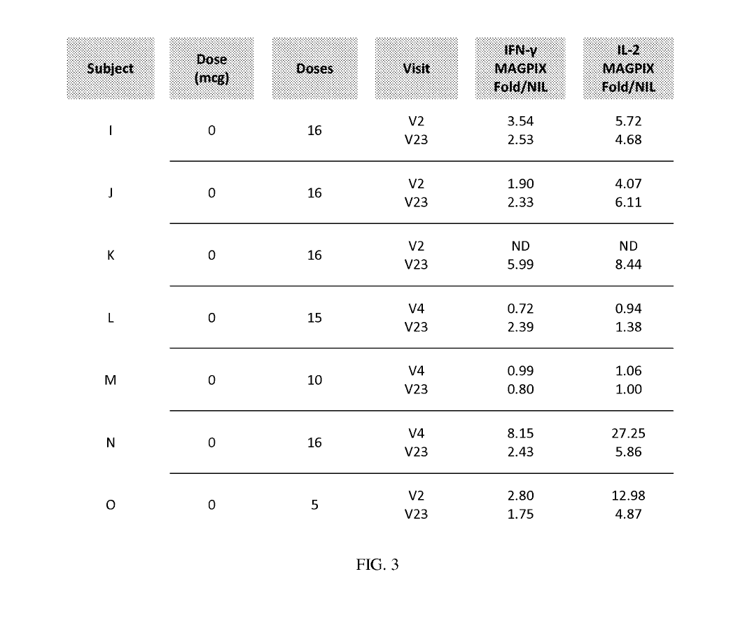

Data from subjects with HLA-DQ2.5+ Celiac disease on a gluten-free diet treated with 150 micrograms of peptide pool 1 were subjected to an oral gluten challenge before the first dose of treatment and after the last dose of treatment. IL-2 was then measured by MAGPIX in whole blood samples from subject after the whole blood was contacted with peptide pool 1 or a negative control. A summary of the IL-2 measurements is shown in FIG. 2, compared to corresponding IFN-γ MAGPIX. In general, the fold change was higher prior to the first dose compared to after the last dose using the IL-2 assay. This fold change difference between first dose and last dose was generally not observed in subjects treated with placebo (FIG. 3). These data further confirm that IL-2 can be used to identify subjects with Celiac disease.

### Example 4. Whole Blood Cytokine Release Stimulated by Gluten Peptides in Seronegative CD Patients Compared to Seronegative Patients with Non-Celiac Gluten Sensitivity with Reduced Intake of Dietary Gluten

### Aim:

To assess gluten-peptide pool stimulated whole blood cytokine release assays for celiac disease (CD) patients negative for CD-specific serology (tTG-IgA and DGP-IgG).

### Endpoints:

Sensitivity and specificity of whole blood cytokine release detected by IL-2 ELISA for CD patients vs non-celiac gluten-sensitive (NCGS) patients who carry HLA-DQ genotypes associated with celiac disease (HLA-DQ2.5+ or DQ2.2+ or DQ8+).

### Patients:

### Inclusion:

(1) Celiac disease on gluten-free diet—diagnosis of CD established and documented according to Expert Clinical Guidelines (e.g. World Gastroenterology Organisation Global Guidelines on Celiac Disease. 2013) who self report being generally compliant with gluten-free diet

### OR

Non-celiac gluten-sensitive—established by normal tTG-IgA serology and/or small bowel histology while regularly consuming gluten who self report being generally compliant with gluten-free diet
(2) No medical contradiction to blood collection by standard venepuncture with a 21G butterfly needle
(3) tTG-IgA (INOVA rhtTG-IgA) and DGP-IgG (INOVA Gliaden II IgG) within the laboratory normal range
(4) Aged 18 or older

### Screening Tests and Information:

EDTA-anticoagulated blood for comprehensive HLA-DQA and HLA-DQB allele determination
Serum tTG-IgA (INOVA rhtTG-IgA) and DGP-IgG (INOVA Gliaden II IgG) assessment
Documentation of medical tests establishing or excluding a diagnosis of celiac disease
Symptoms at diagnosis and current GI symptoms
Duration of gluten-free diet
Co-morbidities (if any)
Medications (if any)

### Age and sex

### Procedure:

Subjects attend a single visit to the trial site for collection of blood to perform:
1. HLA-DQ gene test (Lavender-top EDTA 5 mL, Melbourne Pathology, SONIC)
2. CD serology (Brown-top serum tube 5 mL, Dorevitch Pathology),
3. Whole blood release—subjects will have ONE tube (1 mL blood/tube) for each whole blood incubation condition (9 Quantiferon-GoldTB NIL and 1 MITO tube). In addition, 10 of 30 CD subjects will have 27 additional Cellestis NIL tubes drawn to determine inter- and intra-assay variability (the first 10 CD subjects). After blood is drawn, 0.1 mL volumes of aliquots (listed below) are added by 0.5 mL insulin syringe to NIL tubes containing 1 mL blood, and PBS is added to MITOGEN tube containing 1 mL blood. All Quantiferon tubes are placed in 37° C. incubator. After 24 h incubation, plasma is separated from blood in the Quantiferon tubes and placed in appropriately labeled cryovials then frozen −80° C. Frozen plasma samples are then used for ELISA determination of IL-2.
   Tubes and aliquots are prepared containing one of the following:

### PBS

### PBS+0.5% DMSO,

CEFT 11 ug/mL,
Pool 1—P3 pool 550 μg/mL in PBS (see Example 1 for P3 pool peptides)
Pool 2—P14 pool 275 μM in PBS (see Example 1 for P14 pool peptides)
Pool 3—Total Gluten 110 μg/mL in PBS 0.5% DMSO
Pool 4a—P16 pool 110 μM in PBS
Pool 4b—P16 pool 275 μM in PBS
Pool 4c—P16 pool 550 μM in PBS

P16 pool

| Peptide | Epitope(s) | |
|---|---|---|
| (pE)PFPQPELPYPQP- | PFPQPELPY | (SEQ ID NO: 1122), |
| amide | PQPELPYPQ | (SEQ ID NO: 1123) |
| (SEQ ID NO: 1106) | | |
| (pE)PFPQPEQPFPWQ- | PFPQPEQPF | (SEQ ID NO: 1124), |
| amide | PQPEQPFPW | (SEQ ID NO: 1125) |
| (SEQ ID NO: 1107) | | |
| (pE)EQPIPEQPQPYP- | EQPIPEQPQ | (SEQ ID NO: 1126), |
| amide | PIPEQPQPY | (SEQ ID NO: 1127) |
| (SEQ ID NO: 1108) | | |
| (pE)PFPQPEQPIPVQ- | PFPQPEQPI | (SEQ ID NO: 1128), |
| amide | PQPEQPIPV | (SEQ ID NO: 1129) |
| (SEQ ID NO: 1109) | | |
| (pE)PEQPIPVQPEQS- | EQPIPVQPE | (SEQ ID NO: 1130) |
| amide | | |
| (SEQ ID NO: 1110) | | |
| (pE)PFPQPEQPTPIQ- | PFPQPEQPT | (SEQ ID NO: 1131), |
| amide | PQPEQPTPI | (SEQ ID NO: 1132) |
| (SEQ ID NO: 1111) | | |
| (pE)PEQPTPIQPEQP- | EQPTPIQPE | (SEQ ID NO: 1133) |
| amide | | |
| (SEQ ID NO: 1112) | | |
| (pE)PFPQPEQPFPLQ- | PQPEQPFPL | (SEQ ID NO: 1134) |
| amide | | |
| (SEQ ID NO: 1113) | | |
| (pE)PEQPFPLQPEQP- | EQPFPLQPE | (SEQ ID NO: 1135) |
| amide | | |
| (SEQ ID NO: 1114) | | |
| (pE)GEGSFQPSQENP- | EGSFQPSQE | (SEQ ID NO: 1136) |
| amide | | |
| (SEQ ID NO: 1115) | | |
| (pE)QQGYYPTSPQQS- | QGYYPTSPQ | (SEQ ID NO: 1137) |
| amide | | |
| (SEQ ID NO: 1116) | | |
| (pE)PEQPEQPFPEQP- | EQPEQPFPE | (SEQ ID NO: 1138) |
| amide | | |
| (SEQ ID NO: 1117) | | |
| (pE)PPFSEQEQPVLP- | PFSEQEQPV | (SEQ ID NO: 1139) |
| amide | | |
| (SEQ ID NO: 1118) | | |
| (pE)PYPQPELPYPQP- | PYPQPELPY | (SEQ ID NO: 1140), |
| amide | (PQPELPYPQ) | (SEQ ID NO: 1141) |
| (SEQ ID NO: 1119) | | |
| (pE)EQPFPEQPIPEQ- | EQPFPEQPI | (SEQ ID NO: 1142), |
| amide | PFPEQPIPE | (SEQ ID NO: 1143) |
| (SEQ ID NO: 1120) | | |
| (pE)PQPYPEQPQPFP- | PYPEQPQPF | (SEQ ID NO: 1144), |
| amide | PQPYPEQPQ | (SEQ ID NO: 1145) |
| (SEQ ID NO: 1121) | | |

(pE) = pyroglutamate

Validated ELISAs and/or bead-based multiplex assays will be used for determination of IL-2 and IFN-γ and will be used to establish an upper limit for stimulation index and concentration of each analyte using plasma collected from NCGS who are not genetically susceptible to celiac disease. In the initial analysis, data points will be determined to be elevated or not according to this threshold (e.g. Stimulated blood minus NIL with PBS only >7.2 pg/mL and Stimulated blood/NIL with PBS only >1.25 for IFNγ). Threshold values to optimize sensitivity and specificity differentiating CD vs NCGS will be further refined according receiver operating characteristic (ROC) curve analysis and area under the curve (AUC) analysis. Data from subjects with CD who are excluded because of being seropositive for tTG-IgA or DGP-IgG will be reported and analyzed separately according to the same cutoffs as applied to other subjects.

### Sample Size Estimation

Celiac disease—approximately ⅓ of treated CD subjects show elevated CD-serology and >99% are HLA-DQ2.5+ or DQ8+ or DQ2.2+
NCGS—all have normal CD serology and 60% are HLA-DQ2.5+ or DQ8+ or DQ2.2+
To enroll ˜20 seronegative CD subjects, 30 total should be enrolled
To enroll ˜20 HLA-DQ2.5+ or DQ8+ or DQ2.2+ NCGS subjects, 30 total should enrolled

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of identifying a subject having or at risk for having Celiac disease, the method comprising:
(a) determining a level of IL-2 in a sample comprising a T cell from the subject, which sample has been contacted with a composition comprising at least one gluten peptide; and
(b) assessing whether or not the subject has or is at risk of having Celiac disease.

2. The method of claim 1, wherein the determining step comprises:
(i) contacting the sample comprising the T cell with the composition comprising at least one gluten peptide; and
(ii) measuring the level of IL-2 in the sample comprising the T cell after the contacting.

3. The method of claim 2, wherein measuring the level of IL-2 comprises an enzyme-linked immunosorbent assay (ELISA) or a multiplex bead-based immunoassay.

4. The method of any one of claims 1 to 3, wherein the method further comprises:
(c) comparing the level of IL-2 in the sample with a control level of IL-2.

5. The method of any one of claims 1 to 4, wherein the assessing comprises:
(i) identifying the subject as having or at risk of having Celiac disease if the IL-2 level is elevated compared to a control level of IL-2; or
(ii) not having or not at risk of having Celiac disease if the IL-2 level is reduced compared to the control level of IL-2 or the same as the control level of IL-2.

6. The method of claim 5, wherein the control level of IL-2 is a pre-determined threshold.

7. The method of claim 5, wherein the control level of IL-2 is the level of IL-2 in another sample comprising a T cell obtained from the subject that is not contacted with the composition comprising at least one gluten peptide.

8. The method of any one claims 1 to 7, wherein the sample comprising the T cell is a sample that comprises whole blood or peripheral blood mononuclear cells.

9. The method of any one of claims 1 to 8, wherein the composition comprises at least peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 40), PYPEQPQPF (SEQ ID NO: 46), EGSFQPSQE (SEQ ID NO: 47), QGYYPTSPQ (SEQ ID NO: 16), EQPEQPFPE (SEQ ID NO: 17), EQPFPEQPQ (SEQ ID NO: 41), PFPEQPEQI (SEQ ID NO: 42), PFSEQEQPV (SEQ ID NO: 43), EQPFPEQPI (SEQ ID NO: 20), PFPEQPIPE (SEQ ID NO: 21), PYPQPELPY (SEQ ID NO: 19), PQPELPYPY (SEQ ID NO: 44), and PQPYPEQPQ (SEQ ID NO: 23).

10. The method of any one of claims 1 to 9, wherein the composition comprises at least one of
(a) a first peptide comprising the amino acid sequence PFPQPELPY (SEQ ID NO: 24) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);
(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);
(f) a sixth peptide comprising the amino acid sequence PFPQPEQPF (SEQ ID NO: 3), the amino acid sequence PQPEQPFPL (SEQ ID NO: 1), and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 40);
(h) an eighth peptide comprising the amino acid sequence PYPEQPQPF (SEQ ID NO: 22);
(i) a ninth peptide comprising the amino acid sequence PFPEQPEQI (SEQ ID NO: 42);
(j) a tenth peptide comprising the amino acid sequence EGSFQPSQE (SEQ ID NO: 15);
(k) an eleventh peptide comprising the amino acid sequence QGYYPTSPQ (SEQ ID NO: 16);
(l) a twelfth peptide comprising the amino acid sequence EQPEQPFPE (SEQ ID NO: 17) and the amino acid sequence EQPFPEQPQ (SEQ ID NO: 41);
(m) a thirteenth peptide comprising the amino acid sequence PFSEQEQPV (SEQ ID NO: 18);
(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 19);
(p) a sixteenth peptide comprising the amino acid sequence PFPQPELPY (SEQ ID NO: 1) and the amino acid sequence PQPELPYPY (SEQ ID NO: 44);
(q) a seventeenth peptide comprising the amino acid sequence EQPFPEQPI (SEQ ID NO: 20) and the amino acid sequence PFPEQPIPE (SEQ ID NO: 21); and
(r) an eighteenth peptide comprising the amino acid sequence PQPYPEQPQ (SEQ ID NO: 23) and the amino acid sequence PYPEQPQPF (SEQ ID NO: 22).

11. The method of claim 10, wherein:
(a) the first peptide comprises the amino acid sequence LQPFPQPELPYPQPQ (SEQ ID NO: 84);
(b) the second peptide comprises the amino acid sequence QPFPQPEQPFPWQP (SEQ ID NO: 85);
(c) the third peptide comprises the amino acid sequence PEQPIPEQPQPYPQQ (SEQ ID NO: 86);
(d) the fourth peptide comprises the amino acid sequence QPFPQPEQPIPVQPEQS (SEQ ID NO: 87);
(e) the fifth peptide comprises the amino acid sequence QPFPQPEQPTPIQPEQP (SEQ ID NO: 88);
(f) the sixth peptide comprises the amino acid sequence QPFPQPEQPFPLQPEQP (SEQ ID NO: 89);
(g) the seventh peptide comprises the amino acid sequence QPFPQPEQPFSQQ (SEQ ID NO: 90);
(h) the eighth peptide comprises the amino acid sequence PQPYPEQPQPFPQQ (SEQ ID NO: 91);
(i) the ninth peptide comprises the amino acid sequence QPFPEQPEQIIPQQP (SEQ ID NO: 92);
(j) the tenth peptide comprises the amino acid sequence SGEGSFQPSQENPQ (SEQ ID NO: 93);
(k) the eleventh peptide comprises the amino acid sequence GQQGYYPTSPQQSG (SEQ ID NO: 94);
(l) the twelfth peptide comprises the amino acid sequence PEQPEQPFPEQPQQ (SEQ ID NO: 95);
(m) the thirteenth peptide comprises the amino acid sequence QPPFSEQEQPVLPQ (SEQ ID NO: 96);
(n) the fourteenth peptide comprises the amino acid sequence PEQPFPEQPIPEQPQPYP (SEQ ID NO: 97);
(o) the fifteenth peptide comprises the amino acid sequence QPYPQPELPYPQPQ (SEQ ID NO: 98);
(p) the sixteenth peptide comprises the amino acid sequence QPFPQPELPYPYPQ (SEQ ID NO: 99);
(q) the seventeenth peptide comprises the amino acid sequence PQEQPFPEQPIPEQP (SEQ ID NO: 100); and
(r) the eighteenth peptide comprises the amino acid sequence QPQPYPEQPQPFPQQ (SEQ ID NO: 101).

12. The method of any one of claims 1 to 8, wherein the composition comprises at least one of:
(i) a first peptide comprising the amino acid sequence PFPQPELPY (SEQ ID NO: 1) and the amino acid sequence PQPELPYPQ (SEQ ID NO: 2);
(ii) a second peptide comprising the amino acid sequence PFPQPEQPF (SEQ ID NO: 3) and the amino acid sequence PQPEQPFPW (SEQ ID NO: 4);
(iii) a third peptide comprising the amino acid sequence PIPEQPQPY (SEQ ID NO: 6);
(iv) a fourth peptide comprising the amino acid sequence PFPQPEQPIP (SEQ ID NO: 127) and the amino acid sequence EQPIPVQPE (SEQ ID NO: 9);
(v) a fifth peptide comprising the amino acid sequence PFPQPEQPTPI (SEQ ID NO: 29) and the amino acid sequence EQPTPIQPE (SEQ ID NO: 12);
(vi) a sixth peptide comprising the amino acid sequence PQPEQPFPL (SEQ ID NO: 13) and the amino acid sequence EQPFPLQPE (SEQ ID NO: 14);
(vii) a seventh peptide comprising the amino acid sequence PFPQPEQPF (SEQ ID NO: 3) and the amino acid sequence PQPEQPFSQ (SEQ ID NO: 40);
(viii) an eighth peptide comprising the amino acid sequence PYPEQPQPF (SEQ ID NO: 22);
(ix) a ninth peptide comprising the amino acid sequence PFPEQPEQIIP (SEQ ID NO: 116);
(x) a tenth peptide comprising the amino acid sequence EGSFQPSQE (SEQ ID NO: 15);
(xi) an eleventh peptide comprising the amino acid sequence QGYYPTSPQ (SEQ ID NO: 16);
(xii) a twelfth peptide comprising the amino acid sequence EQPEQPFPEQPQ (SEQ ID NO: 119);
(xiii) a thirteenth peptide comprising the amino acid sequence PFSEQEQPV (SEQ ID NO: 18);
(xiv) a fourteenth peptide comprising the amino acid sequence EQPFPEQPI (SEQ ID NO: 20) and PIPEQPQPY (SEQ ID NO: 6);
(xv) a fifteenth peptide comprising the amino acid sequence PQPELPYPQ (SEQ ID NO: 2) and the amino acid sequence PYPQPELPY (SEQ ID NO: 19);
(xvi) a sixteenth peptide comprising the amino acid sequence PFPQPELPY (SEQ ID NO: 1) and the amino acid sequence PQPELPYPY (SEQ ID NO: 44); and
(xvii) a seventeenth peptide comprising the amino acid sequence EQPFPEQPI (SEQ ID NO: 20).

13. The method of claim 12, wherein
(i) the first peptide comprises the amino acid sequence LQPFPQPELPYPQPQ (SEQ ID NO: 84);
(ii) the second peptide comprises the amino acid sequence QPFPQPEQPFPWQP (SEQ ID NO: 85);
(iii) the third peptide comprises the amino acid sequence PEQPIPEQPQPYPQQ (SEQ ID NO: 86);
(iv) the fourth peptide comprises the amino acid sequence QPFPQPEQPIPVQPEQS (SEQ ID NO: 87);
(v) the fifth peptide comprises the amino acid sequence QPFPQPEQPTPIQPEQP (SEQ ID NO: 88);
(vi) the sixth peptide comprises the amino acid sequence QPFPQPEQPFPLQPEQP (SEQ ID NO: 89);
(vii) the seventh peptide comprises the amino acid sequence QPFPQPEQPFSQQ (SEQ ID NO: 90);
(viii) the eighth peptide comprises the amino acid sequence PQPYPEQPQPFPQQ (SEQ ID NO: 91);
(ix) the ninth peptide comprises the amino acid sequence QPFPEQPEQIIPQQP (SEQ ID NO: 92);
(x) the tenth peptide comprises the amino acid sequence SGEGSFQPSQENPQ (SEQ ID NO: 93);
(xi) the eleventh peptide comprises the amino acid sequence GQQGYYPTSPQQSG (SEQ ID NO: 94);
(xii) the twelfth peptide comprises the amino acid sequence PEQPEQPFPEQPQQ (SEQ ID NO: 95);
(xiii) the thirteenth peptide comprises the amino acid sequence QPPFSEQEQPVLPQ (SEQ ID NO: 96);
(xiv) the fourteenth peptide comprises the amino acid sequence PEQPFPEQPIPEQPQPYP (SEQ ID NO: 97);
(xv) the fifteenth peptide comprises the amino acid sequence QPYPQPELPYPQPQ (SEQ ID NO: 98);
(xvi) the sixteenth peptide comprises the amino acid sequence QPFPQPELPYPYPQ (SEQ ID NO: 99); and
(xvii) a seventeenth peptide comprises the amino acid sequence EQPFPEQPI (SEQ ID NO: 20).

14. The method of any one of claims 10 to 13, wherein the composition comprises the first, second, and third peptide.

15. The method of any one of claims 10 to 13, wherein the composition comprises the first, second, third, fourth, fifth, sixth, seventh, eighth, ninth, tenth, eleventh, twelfth, and thirteenth peptides.

16. The method of any one of claims 10 to 13, wherein the composition comprises the second, fourth, fifth, sixth, seventh, eighth, ninth, tenth, eleventh, twelfth, thirteenth, fourteenth, fifteenth, and sixteenth peptides.

17. The method of any one of claims 1 to 9, wherein the composition comprises at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7) and the amino acid sequence PQPEQPIPV (SEQ ID NO: 8);
(e) a fifth peptide comprising the amino acid sequence EQPIPVQPE (SEQ ID NO: 9);
(f) a sixth peptide comprising the amino acid sequence PFPQPEQPT (SEQ ID NO: 10) and the amino acid sequence PQPEQPTPI (SEQ ID NO: 11);
(g) a seventh peptide comprising the amino acid sequence EQPTPIQPE (SEQ ID NO: 12);
(h) an eighth peptide comprising the amino acid sequence PQPEQPFPL (SEQ ID NO: 13);
(i) a ninth peptide comprising the amino acid sequence EQPFPLQPE (SEQ ID NO: 14);
(j) a tenth peptide comprising the amino acid sequence EGSFQPSQE (SEQ ID NO: 15);
(k) a eleventh peptide comprising the amino acid sequence QGYYPTSPQ (SEQ ID NO: 16);
(l) a twelfth peptide comprising the amino acid sequence EQPEQPFPE (SEQ ID NO: 17);
(m) a thirteenth peptide comprising the amino acid sequence PFSEQEQPV (SEQ ID NO: 18);
(n) a fourteenth peptide comprising the amino acid sequence PYPQPELPY (SEQ ID NO: 19);
(o) a fifteenth peptide comprising the amino acid sequence EQPFPEQPI (SEQ ID NO: 20) and the amino acid sequence PFPEQPIPE (SEQ ID NO: 21); and
(p) a sixteenth peptide comprising the amino acid sequence PYPEQPQPF (SEQ ID NO: 22) and the amino acid sequence PQPYPEQPQ (SEQ ID NO: 23).

18. The method of claim 17, wherein
(a) the first peptide comprises the amino acid sequence PFPQPELPYPQP (SEQ ID NO: 24);
(b) the second peptide comprises the amino acid sequence PFPQPEQPFPWQ (SEQ ID NO: 25);
(c) the third peptide comprises the amino acid sequence EQPIPEQPQPYP (SEQ ID NO: 26);
(d) the fourth peptide comprises the amino acid sequence PFPQPEQPIPVQ (SEQ ID NO: 27);
(e) the fifth peptide comprises the amino acid sequence PEQPIPVQPEQS (SEQ ID NO: 28);
(f) the sixth peptide comprises the amino acid sequence PFPQPEQPTPIQ (SEQ ID NO: 29);
(g) the seventh peptide comprises the amino acid sequence PEQPTPIQPEQP (SEQ ID NO: 30);
(h) the eighth peptide comprises the amino acid sequence PFPQPEQPFPLQ (SEQ ID NO: 31);
(i) the ninth peptide comprises the amino acid sequence PEQPFPLQPEQP (SEQ ID NO: 32);
(j) the tenth peptide comprises the amino acid sequence GEGSFQPSQENP (SEQ ID NO: 33);
(k) the eleventh peptide comprises the amino acid sequence QQGYYPTSPQQS (SEQ ID NO: 34);
(l) the twelfth peptide comprises the amino acid sequence PEQPEQPFPEQP (SEQ ID NO: 35);
(m) the thirteenth peptide comprises the amino acid sequence PPFSEQEQPVLP (SEQ ID NO: 36);
(n) the fourteenth peptide comprises the amino acid sequence PYPQPELPYPQP (SEQ ID NO: 37);
(o) the fifteenth peptide comprises the amino acid sequence EQPFPEQPIPEQ (SEQ ID NO: 38); and
(p) the sixteenth peptide comprises the amino acid sequence PQPYPEQPQPFP (SEQ ID NO: 39).

19. The method of claim 18, wherein the composition comprises the first, second, third, fourth, fifth, sixth, seventh, eighth, ninth, tenth, eleventh, twelfth, thirteenth, fourteenth, fifteenth, and sixteenth peptides.

20. The method of any one of claims 9-19, wherein at least one of the peptides comprises an N-terminal pyroglutamate and/or a C-terminal amide group.

21. The method of claim 20, wherein each of the peptides comprises an N-terminal pyroglutamate and/or a C-terminal amide group.

22. The method of any one of claims 9-21, wherein each of the peptides is independently between 8 to 50 amino acids in length.

23. The method of any one of claims 1 to 22, wherein the method further comprises treating the subject if identified as having or at risk of having Celiac disease or providing information to the subject about a treatment.

24. The method of any one of claims 1 to 23, where the method further comprises a step of recommending a gluten-free diet if the subject is identified as having or at risk of having Celiac disease or providing information to the subject about such a diet.

25. The method of any one of claims 1 to 24, wherein the method further comprises performing other testing.

26. The method of any one of claim 25, wherein the other testing comprises performing a serology assay, genotyping, and/or an intestinal biopsy.

27. The method of claims 1 to 26, wherein the subject is HLA-DQ2.5 positive, HLA-DQ2.2 positive and/or HLA-DQ8 positive.

28. The method of claim 27, wherein the subject is HLA-DQ2.5 positive.

29. The method of any one of claims 1 to 28, wherein the method further comprises administering a composition comprising wheat, rye, and/or barley, or a composition comprising a gluten peptide, to the subject at least once a day for one day.

30. The method of any one of claims 1 to 29, wherein the method further comprises administering a composition comprising wheat, rye, and/or barley to the subject at least once a day for two days.

31. The method of any one of claims 1 to 29, wherein the subject has not undergone a gluten challenge within 1 week of the sample being obtained from the subject.

32. The method of any one of the preceding claims, wherein the subject has a level of IFN-gamma that is reduced or the same as a control level of IFN-gamma.

33. The method of claim 32, wherein the level of IFN-gamma is not statistically significantly higher than the control level of IFN-gamma.

34. The method of claim 32 or 33, wherein the control level of IFN-gamma is 7.2 pg/mL.

35. The method of any one of the preceding claims, wherein the subject is on a diet that contains gluten.

36. A kit, comprising a binding partner for IL-2 and a composition comprising at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);
(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);
(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 40);
(h) an eighth peptide comprising the amino acid sequence PYPEQPQPF (SEQ ID NO: 22);
(i) a ninth peptide comprising the amino acid sequence PFPEQPEQI (SEQ ID NO: 42);
(j) a tenth peptide comprising the amino acid sequence EGSFQPSQE (SEQ ID NO: 15);
(k) an eleventh peptide comprising the amino acid sequence QGYYPTSPQ (SEQ ID NO: 16);
(l) a twelfth peptide comprising the amino acid sequence EQPEQPFPE (SEQ ID NO: 17) and the amino acid sequence EQPFPEQPQ (SEQ ID NO: 41);
(m) a thirteenth peptide comprising the amino acid sequence PFSEQEQPV (SEQ ID NO: 18);
(n) a fourteenth peptide comprising the amino acid sequence EQPFPEQPI (SEQ ID NO: 20), the amino acid sequence PFPEQPIPE (SEQ ID NO: 21), the amino acid sequence EQPIPEQPQ (SEQ ID NO: 5), and the amino acid sequence PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 19);
(p) a sixteenth peptide comprising the amino acid sequence PFPQPELPY (SEQ ID NO: 1) and the amino acid sequence PQPELPYPY (SEQ ID NO: 44);
(q) a seventeenth peptide comprising the amino acid sequence EQPFPEQPI (SEQ ID NO: 20) and the amino acid sequence PFPEQPIPE (SEQ ID NO: 21); and
(r) an eighteenth peptide comprising the amino acid sequence PQPYPEQPQ (SEQ ID NO: 23) and the amino acid sequence PYPEQPQPF (SEQ ID NO: 22).

37. The kit of claim 36, wherein
(a) the first peptide comprises the amino acid sequence LQPFPQPELPYPQPQ (SEQ ID NO: 84);
(b) the second peptide comprises the amino acid sequence QPFPQPEQPFPWQP (SEQ ID NO: 85);
(c) the third peptide comprises the amino acid sequence PEQPIPEQPQPYPQQ (SEQ ID NO: 86);
(d) the fourth peptide comprises the amino acid sequence QPFPQPEQPIPVQPEQS (SEQ ID NO: 87);
(e) the fifth peptide comprises the amino acid sequence QPFPQPEQPTPIQPEQP (SEQ ID NO: 88);
(f) the sixth peptide comprises the amino acid sequence QPFPQPEQPFPLQPEQP (SEQ ID NO: 89);
(g) the seventh peptide comprises the amino acid sequence QPFPQPEQPFSQQ (SEQ ID NO: 90);
(h) the eighth peptide comprises the amino acid sequence PQPYPEQPQPFPQQ (SEQ ID NO: 91);
(i) the ninth peptide comprises the amino acid sequence QPFPEQPEQIIPQQP (SEQ ID NO: 92);
(j) the tenth peptide comprises the amino acid sequence SGEGSFQPSQENPQ (SEQ ID NO: 93);
(k) the eleventh peptide comprises the amino acid sequence GQQGYYPTSPQQSG (SEQ ID NO: 94);
(l) the twelfth peptide comprises the amino acid sequence PEQPEQPFPEQPQQ (SEQ ID NO: 95);
(m) the thirteenth peptide comprises the amino acid sequence QPPFSEQEQPVLPQ (SEQ ID NO: 96);
(n) the fourteenth peptide comprises the amino acid sequence PEQPFPEQPIPEQPQPYP (SEQ ID NO: 97);
(o) the fifteenth peptide comprises the amino acid sequence QPYPQPELPYPQPQ (SEQ ID NO: 98);
(p) the sixteenth peptide comprises the amino acid sequence QPFPQPELPYPYPQ (SEQ ID NO: 99);
(q) the seventeenth peptide comprises the amino acid sequence PQEQPFPEQPIPEQP (SEQ ID NO: 100); and
(r) the eighteenth peptide comprises the amino acid sequence QPQPYPEQPQPFPQQ (SEQ ID NO: 101).

38. The kit of claim 36 or 37, wherein the composition comprises the first, second, and third peptides.

39. The kit of claim 38, wherein each of the peptides are present in an amount of 5 ug/mL in the composition.

40. The kit of claim 38, wherein each of the peptides are present in an amount of 10 ug/mL in the composition.

41. The kit of claim 38, wherein each of the peptides are present in an amount of 20 ug/mL in the composition.

42. The kit of claim 38, wherein each of the peptides are present in an amount of 50 ug/mL in the composition.

43. The kit of claim 38, wherein each of the peptides are present in an amount of 5 uM in the composition.

44. The kit of claim 38, wherein each of the peptides are present in an amount of 10 uM in the composition.

45. The kit of claim 38, wherein each of the peptides are present in an amount of 25 uM in the composition.

46. The kit of claim 38, wherein each of the peptides are present in an amount of 50 uM in the composition.

47. The kit of claim 36 or 37, wherein the composition comprises:
the first, second, third, fourth, fifth, sixth, seventh, eighth, ninth, tenth, eleventh, twelfth, and thirteenth peptides;
the second, fourth, fifth, sixth, seventh, eighth, ninth, tenth, eleventh, twelfth, thirteenth, fourteenth, fifteenth, and sixteenth peptides; or
the first, second, third, fourth, fifth, sixth, tenth, eleventh, twelfth, thirteenth, fifteenth, seventeenth, and eighteenth peptides.

48. The kit of claim 47, wherein each of the peptides are present in an amount of 2.5 ug/mL in the composition.

49. The kit of claim 47, wherein each of the peptides are present in an amount of 5 ug/mL in the composition.

50. The kit of claim 47, wherein each of the peptides are present in an amount of 10 ug/mL in the composition.

51. The kit of claim 47, wherein each of the peptides are present in an amount of 25 ug/mL in the composition.

52. The kit of claim 47, wherein each of the peptides are present in an amount of 5 uM in the composition.

53. The kit of claim 47, wherein each of the peptides are present in an amount of 10 uM in the composition.

54. The kit of claim 47, wherein each of the peptides are present in an amount of 25 uM in the composition.

55. The kit of claim 47, wherein each of the peptides are present in an amount of 50 uM in the composition.

56. The kit of any one of claims 36-55, wherein each of the peptides comprises an N-terminal pyroglutamate and/or a C-terminal amide group.

57. A kit, comprising a binding partner for IL-2 and a composition comprising at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7) and the amino acid sequence PQPEQPIPV (SEQ ID NO: 8);
(e) a fifth peptide comprising the amino acid sequence EQPIPVQPE (SEQ ID NO: 9);
(f) a sixth peptide comprising the amino acid sequence PFPQPEQPT (SEQ ID NO: 10) and the amino acid sequence PQPEQPTPI (SEQ ID NO: 11);
(g) a seventh peptide comprising the amino acid sequence EQPTPIQPE (SEQ ID NO: 12);
(h) an eighth peptide comprising the amino acid sequence PQPEQPFPL (SEQ ID NO: 13);
(i) a ninth peptide comprising the amino acid sequence EQPFPLQPE (SEQ ID NO: 14);
(j) a tenth peptide comprising the amino acid sequence EGSFQPSQE (SEQ ID NO: 15);
(k) a eleventh peptide comprising the amino acid sequence QGYYPTSPQ (SEQ ID NO: 16);
(l) a twelfth peptide comprising the amino acid sequence EQPEQPFPE (SEQ ID NO: 17);
(m) a thirteenth peptide comprising the amino acid sequence PFSEQEQPV (SEQ ID NO: 18);
(n) a fourteenth peptide comprising the amino acid sequence PYPQPELPY (SEQ ID NO: 19);
(o) a fifteenth peptide comprising the amino acid sequence EQPFPEQPI (SEQ ID NO: 20) and the amino acid sequence PFPEQPIPE (SEQ ID NO: 21); and
(p) a sixteenth peptide comprising the amino acid sequence PYPEQPQPF (SEQ ID NO: 22) and the amino acid sequence PQPYPEQPQ (SEQ ID NO: 23).

58. The kit of claim 58, wherein
(a) the first peptide comprises the amino acid sequence PFPQPELPYPQP (SEQ ID NO: 24);
(b) the second peptide comprises the amino acid sequence PFPQPEQPFPWQ (SEQ ID NO: 25);
(c) the third peptide comprises the amino acid sequence EQPIPEQPQPYP (SEQ ID NO: 26);
(d) the fourth peptide comprises the amino acid sequence PFPQPEQPIPVQ (SEQ ID NO: 27);
(e) the fifth peptide comprises the amino acid sequence PEQPIPVQPEQS (SEQ ID NO: 28);
(f) the sixth peptide comprises the amino acid sequence PFPQPEQPTPIQ (SEQ ID NO: 29);
(g) the seventh peptide comprises the amino acid sequence PEQPTPIQPEQP (SEQ ID NO: 30);
(h) the eighth peptide comprises the amino acid sequence PFPQPEQPFPLQ (SEQ ID NO: 31);
(i) the ninth peptide comprises the amino acid sequence PEQPFPLQPEQP (SEQ ID NO: 32);
(j) the tenth peptide comprises the amino acid sequence GEGSFQPSQENP (SEQ ID NO: 33);
(k) the eleventh peptide comprises the amino acid sequence QQGYYPTSPQQS (SEQ ID NO: 34);
(l) the twelfth peptide comprises the amino acid sequence PEQPEQPFPEQP (SEQ ID NO: 35);
(m) the thirteenth peptide comprises the amino acid sequence PPFSEQEQPVLP (SEQ ID NO: 36);
(n) the fourteenth peptide comprises the amino acid sequence PYPQPELPYPQP (SEQ ID NO: 37);
(o) the fifteenth peptide comprises the amino acid sequence EQPFPEQPIPEQ (SEQ ID NO: 38); and
(p) the sixteenth peptide comprises the amino acid sequence PQPYPEQPQPFP (SEQ ID NO: 39).

59. The kit of claim 57 or 58, wherein the composition comprises the first, second, third, fourth, fifth, sixth, seventh, eighth, ninth, tenth, eleventh, twelfth, thirteenth, fourteenth, fifteenth, and sixteenth peptides.

60. The kit of claim 59, wherein each of the peptides are present in an amount of 5 ug/mL in the composition.

61. The kit of claim 59, wherein each of the peptides are present in an amount of 10 ug/mL in the composition.

62. The kit of claim 59, wherein each of the peptides are present in an amount of 20 ug/mL in the composition.

63. The kit of claim 59, wherein each of the peptides are present in an amount of 50 ug/mL in the composition.

64. The kit of claim 59, wherein each of the peptides are present in an amount of 5 uM in the composition.

65. The kit of claim 59, wherein each of the peptides are present in an amount of 10 uM in the composition.

66. The kit of claim 59, wherein each of the peptides are present in an amount of 25 uM in the composition.

67. The kit of claim 59, wherein each of the peptides are present in an amount of 50 uM in the composition.

68. The kit of any one of claims 57-67, wherein each of the peptides comprises an N-terminal pyroglutamate and/or a C-terminal amide group.

69. A kit, comprising a binding partner for IL-2 and a composition comprising at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence PIPEQPQPY (SEQ ID NO: 6);
(d) a fourth peptide comprising the amino acid sequence PFPQPEQPIP (SEQ ID NO: 127) and the amino acid sequence EQPIPVQPE (SEQ ID NO: 9);
(e) a fifth peptide comprising the amino acid sequence PFPQPEQPTPI (SEQ ID NO: 29) and the amino acid sequence EQPTPIQPE (SEQ ID NO: 12);
(f) a sixth peptide comprising the amino acid sequence PQPEQPFPL (SEQ ID NO: 13) and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 40);
(h) an eighth peptide comprising the amino acid sequence PYPEQPQPF (SEQ ID NO: 22);
(i) a ninth peptide comprising the amino acid sequence PFPEQPEQIIP (SEQ ID NO: 116);
(j) a tenth peptide comprising the amino acid sequence EGSFQPSQE (SEQ ID NO: 15);
(k) an eleventh peptide comprising the amino acid sequence QGYYPTSPQ (SEQ ID NO: 16);
(l) a twelfth peptide comprising the amino acid sequence EQPEQPFPEQPQ (SEQ ID NO: 119);
(m) a thirteenth peptide comprising the amino acid sequence PFSEQEQPV (SEQ ID NO: 18);
(n) a fourteenth peptide comprising the amino acid sequence EQPFPEQPI (SEQ ID NO: 20) and PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 19);
(p) a sixteenth peptide comprising the amino acid sequence PFPQPELPY (SEQ ID NO: 1) and the amino acid sequence PQPELPYPY (SEQ ID NO: 44); and
(q) a seventeenth peptide comprising the amino acid sequence EQPFPEQPI (SEQ ID NO: 20).

70. The kit of claim 69, wherein
(a) the first peptide comprises the amino acid sequence LQPFPQPELPYPQPQ (SEQ ID NO: 84);
(b) the second peptide comprises the amino acid sequence QPFPQPEQPFPWQP (SEQ ID NO: 85);
(c) the third peptide comprises the amino acid sequence PEQPIPEQPQPYPQQ (SEQ ID NO: 86);
(d) the fourth peptide comprises the amino acid sequence QPFPQPEQPIPVQPEQS (SEQ ID NO: 87);
(e) the fifth peptide comprises the amino acid sequence QPFPQPEQPTPIQPEQP (SEQ ID NO: 88);
(f) the sixth peptide comprises the amino acid sequence QPFPQPEQPFPLQPEQP (SEQ ID NO: 89);
(g) the seventh peptide comprises the amino acid sequence QPFPQPEQPFSQQ (SEQ ID NO: 90);
(h) the eighth peptide comprises the amino acid sequence PQPYPEQPQPFPQQ (SEQ ID NO: 91);
(i) the ninth peptide comprises the amino acid sequence QPFPEQPEQIIPQQP (SEQ ID NO: 92);
(j) the tenth peptide comprises the amino acid sequence SGEGSFQPSQENPQ (SEQ ID NO: 93);
(k) the eleventh peptide comprises the amino acid sequence GQQGYYPTSPQQSG (SEQ ID NO: 94);
(l) the twelfth peptide comprises the amino acid sequence PEQPEQPFPEQPQQ (SEQ ID NO: 95);
(m) the thirteenth peptide comprises the amino acid sequence QPPFSEQEQPVLPQ (SEQ ID NO: 96);
(n) the fourteenth peptide comprises the amino acid sequence PEQPFPEQPIPEQPQPYP (SEQ ID NO: 97);
(o) the fifteenth peptide comprises the amino acid sequence QPYPQPELPYPQPQ (SEQ ID NO: 98);
(p) the sixteenth peptide comprises the amino acid sequence QPFPQPELPYPYPQ (SEQ ID NO: 99); and
(q) the seventeenth peptide comprises the amino acid sequence EQPFPEQPI (SEQ ID NO: 20).

71. The kit of claim 69 or 70, wherein the composition comprises the first, second, and third peptides.

72. The kit of claim 69 or 70, wherein the composition comprises the first, second, third, fourth, fifth, sixth, seventh, eighth, ninth, tenth, eleventh, twelfth, and thirteenth peptides or the composition comprises the second, fourth, fifth, sixth, seventh, eighth, ninth, tenth, eleventh, twelfth, thirteenth, fourteenth, fifteenth, and sixteenth peptides.

73. The kit of any one of claims 69-72, wherein each of the peptides comprises an N-terminal pyroglutamate and/or a C-terminal amide group.

74. The kit of any one of claims 36-73, wherein the kit further comprises a binding partner for IFN-γ and/or IP-10.
